SEQUENCE LISTING (158 sequences)

SEQ ID NO: 1 - DNA, 3650 bp, synthetic construct
  misc_feature 1..3650: note = chimeric cynomolgus-human EGFR encoding construct
  mat_peptide 82..3639
  source 1..3650: mol_type = other DNA
  CDS 1..3639
gctagcaccatggggcccagcggcaccgccggcgccgccctgctggccctgctggccgccctgtgccccgccagccgggccctggaggagaagaaggtgtgccagggcaccagcaacaagctgacccagctgggcaccttcgaggaccacttcctgagcctgcagcggatgttcaacaactgcgaggtggtgctgggcaacctggagatcacctacgtgcagcggaactacgacctgagcttcctgaagaccatccaggaggtggccggctacgtgctgatcgccctgaacaccgtggagcggatccccctggagaacctgcagatcatccggggcaacatgtactacgagaacagctacgccctggccgtgctgagcaactacgacgccaacaagaccggcctgaaggagctgcccatgcggaacctgcaggagatcctgcacggcgccgtgcggttcagcaacaaccccgccctgtgcaacgtggagagcatccagtggcgggacatcgtgagcagcgagttcctgagcaacatgagcatggacttccagaaccacctgggcagctgccagaagtgcgaccccagctgccccaacggcagctgctggggcgccggcgaggagaactgccagaagctgaccaagatcatctgcgcccagcagtgcagcggccggtgccggggcaagagccccagcgactgctgccacaaccagtgcgccgccggctgcaccggcccccgggagagcgactgcctggtgtgccggaagttccgggacgaggccacctgcaaggacacctgcccccccctgatgctgtacaaccccaccacctaccagatggacgtgaaccccgagggcaagtacagcttcggcgccacctgcgtgaagaagtgcccccggaactacgtggtgaccgaccacggcagctgcgtgcgggcctgcggcgccgacagctacgagatggaggaggacggcgtgcggaagtgcaagaagtgcgagggcccctgccggaaggtgtgcaacggcatcggcatcggcgagttcaaggacaccctgagcatcaacgccaccaacatcaagcacttcaagaactgcaccagcatcagcggcgacctgcacatcctgcccgtggccttccggggcgacagcttcacccacaccccccccctggacccccaggagctggacatcctgaagaccgtgaaggagatcaccggcttcctgctgatccaggcctggcccgagaaccggaccgacctgcacgccttcgagaacctggagatcatccggggccggaccaagcagcacggccagttcagcctggccgtggtgagcctgaacatcaccagcctgggcctgcggagcctgaaggagatcagcgacggcgacgtgatcatcagcggcaacaagaacctgtgctacgccaacaccatcaactggaagaagctgttcggcaccagcagccagaagaccaagatcatcagcaaccggggcgagaacagctgcaaggccaccggccaggtgtgccacgccctgtgcagccccgagggctgctggggccccgagccccgggactgcgtgagctgccagaacgtgagccggggccgggagtgcgtggacaagtgcaacatcctggagggcgagccccgggagttcgtggagaacagcgagtgcatccagtgccaccccgagtgcctgccccaggtgatgaacatcacctgcaccggccggggccccgacaactgcatccagtgcgcccactacatcgacggcccccactgcgtgaagacctgccccgccggcgtgatgggcgagaacaacaccctggtgtggaagtacgccgacgccggccacgtgtgccacctgtgccaccccaactgcacctacggctgcaccggccccggcctggagggctgcgcccggaacggccccaagatccccagcatcgccaccggcatgctgggcgccctgctgctgctgctggtggtggccctgggcatcggcctgttcatgcggcggcggcacatcgtgcggaagcggaccctgcggcggctgctgcaggagcgggagctggtggagcccctgacccccagcggcgaggcccccaaccaggccctgctgcggatcctgaaggagaccgagttcaagaagatcaaggtgctgggcagcggcgccttcggcaccgtgtacaagggcctgtggatccccgagggcgagaaggtgaagatccccgtggccatcaaggagctgcgggaggccaccagccccaaggccaacaaggagatcctggacgaggcctacgtgatggccagcgtggacaacccccacgtgtgccggctgctgggcatctgcctgaccagcaccgtgcagctgatcacccagctgatgcccttcggctgcctgctggactacgtgcgggagcacaaggacaacatcggcagccagtacctgctgaactggtgcgtgcagatcgccaagggcatgaactacctggaggaccggcggctggtgcaccgggacctggccgcccggaacgtgctggtgaagaccccccagcacgtgaagatcaccgacttcggcctggccaagctgctgggcgccgaggagaaggagtaccacgccgagggcggcaaggtgcccatcaagtggatggccctggagagcatcctgcaccggatctacacccaccagagcgacgtgtggagctacggcgtgaccgtgtgggagctgatgaccttcggcagcaagccctacgacggcatccccgccagcgagatcagcagcatcctggagaagggcgagcggctgccccagccccccatctgcaccatcgacgtgtacatgatcatggtgaagtgctggatgatcgacgccgacagccggcccaagttccgggagctgatcatcgagttcagcaagatggcccgggacccccagcggtacctggtgatccagggcgacgagcggatgcacctgcccagccccaccgacagcaacttctaccgggccctgatggacgaggaggacatggacgacgtggtggacgccgacgagtacctgatcccccagcagggcttcttcagcagccccagcaccagccggacccccctgctgagcagcctgagcgccaccagcaacaacagcaccgtggcctgcatcgaccggaacggcctgcagagctgccccatcaaggaggacagcttcctgcagcggtacagcagcgaccccaccggcgccctgaccgaggacagcatcgacgacaccttcctgcccgtgcccgagtacatcaaccagagcgtgcccaagcggcccgccggcagcgtgcagaaccccgtgtaccacaaccagcccctgaaccccgcccccagccgggacccccactaccaggacccccacagcaccgccgtgggcaaccccgagtacctgaacaccgtgcagcccacctgcgtgaacagcaccttcgacagccccgcccactgggcccagaagggcagccaccagatcagcctggacaaccccgactaccagcaggacttcttccccaaggaggccaagcccaacggcatcttcaagggcagcaccgccgagaacgccgagtacctgcgggtggccccccagagcagcgagttcatcggcgcctgagcggccgc

SEQ ID NO: 2 - protein, 1213 aa, synthetic construct
  REGION 1..1213: note = Synthetic Construct
ASTMGPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSEFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDTLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSSQKTKIISNRGENSCKATGQVCHALCSPEGCWGPEPRDCVSCQNVSRGRECVDKCNILEGEPREFVENSECIQCHPECLPQVMNITCTGRGPDNCIQCAHYIDGPHCVKTCPAGVMGENNTLVWKYADAGHVCHLCHPNCTYGCTGPGLEGCARNGPKIPSIATGMLGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 3 - protein, 617 aa, synthetic construct
  REGION 1..617: note = EGFR HER3 swap varII ECD
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPS

SEQ ID NO: 4 - protein, 622 aa, synthetic construct
  REGION 1..622: note = EGFR HER3 swap varIII ECD
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPS

SEQ ID NO: 5 - protein, 619 aa, synthetic construct
  REGION 1..619: note = EGFR HER3 swap varIV ECD
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVEECFSCHPECQPMEGTATCNGSGSDTCAQCAHFRDGPHCVSSCPHGVMGENNTLVWKYADAGHVCHLCHPNCTYGCTGPGLEGCPTNGPKIPS

SEQ ID NO: 6 - DNA, 375 bp, synthetic construct
  misc_feature 1..375: note = MF4280: heavy chain variable region sequence of an EGFR binding antibody
  source 1..375: mol_type = other DNA
  CDS 1..375
caggtgcagctggtgcagtctggggctgaggtgaagaagcctggggcctcagtgaaggtctcctgcaaggtttccggatacaccctcactgaattatccatgcactgggtgcgacaggctcctggtaaagggcttgaatggatgggaggctttgatcctgagtatggtaaaacattcttcgcacagaacttccagggcagagtcaccatgaccgaggacacatctgcagacacagcctacatggagctaagcagcctgagatctgaggacacggccgtgtattactgtgcaacagaggggtattatgagactactacttattactacaacctttttgactcctggggccagggaaccctggtcaccgtctcaagc

SEQ ID NO: 7 - protein, 125 aa, synthetic construct
  REGION 1..125: note = Synthetic Construct
QVQLVQSGAEVKKPGASVKVSCKVSGYTLTELSMHWVRQAPGKGLEWMGGFDPEYGKTFFAQNFQGRVTMTEDTSADTAYMELSSLRSEDTAVYYCATEGYYETTTYYYNLFDSWGQGTLVTVSS

SEQ ID NO: 8 - protein, 30 aa, synthetic construct
  REGION 1..30: note = MF4280 FR1
QVQLVQSGAEVKKPGASVKVSCKVSGYTLT

SEQ ID NO: 9 - protein, 5 aa, synthetic construct
  REGION 1..5: note = MF4280 CDR1
ELSMH

SEQ ID NO: 10 - protein, 14 aa, synthetic construct
  REGION 1..14: note = MF4280 FR2
WVRQAPGKGLEWMG

SEQ ID NO: 11 - protein, 17 aa, synthetic construct
  REGION 1..17: note = MF4280 CDR2
GFDPEYGKTFFAQNFQG

SEQ ID NO: 12 - protein, 32 aa, synthetic construct
  REGION 1..32: note = MF4280 FR3
RVTMTEDTSADTAYMELSSLRSEDTAVYYCAT

SEQ ID NO: 13 - protein, 16 aa, synthetic construct
  REGION 1..16: note = MF4280 CDR3
EGYYETTTYYYNLFDS

SEQ ID NO: 14 - protein, 11 aa, synthetic construct
  REGION 1..11: note = MF4280 FR4
WGQGTLVTVSS

SEQ ID NO: 15 - DNA, 360 bp, synthetic construct
  misc_feature 1..360: note = MF3998: heavy chain variable region sequence of an EGFR binding antibody
  source 1..360: mol_type = other DNA
  CDS 1..360
caggtgcagctggtgcagtctgggtctgagttgaagaagcctggggcctcagtgaaggtttcctgcaaggcttctggatacaccttcactaacaatgccataaattgggtgcgacaggcccctggacaagggcttgagtggatgggatggatcaacaccatcactggggacccaacgtatgcccagggcttcacaggacggtttgtcttctccttggacacctctgtcagcacggcatatctgcagatcagcagcctgaaggctgaggacactggcgtgtattactgtgcgagagaggaatttttggagtggttattctttgactactggggccagggaaccctggtcaccgtctcaagc

SEQ ID NO: 16 - protein, 120 aa, synthetic construct
  REGION 1..120: note = Synthetic Construct
QVQLVQSGSELKKPGASVKVSCKASGYTFTNNAINWVRQAPGQGLEWMGWINTITGDPTYAQGFTGRFVFSLDTSVSTAYLQISSLKAEDTGVYYCAREEFLEWLFFDYWGQGTLVTVSS

SEQ ID NO: 17 - protein, 30 aa, synthetic construct
  REGION 1..30: note = MF3998 FR1
QVQLVQSGSELKKPGASVKVSCKASGYTFT

SEQ ID NO: 18 - protein, 5 aa, synthetic construct
  REGION 1..5: note = MF3998 CDR1
NNAIN

SEQ ID NO: 19 - protein, 14 aa, synthetic construct
  REGION 1..14: note = MF3998 FR2
WVRQAPGQGLEWMG

SEQ ID NO: 20 - protein, 17 aa, synthetic construct
  REGION 1..17: note = MF3998 CDR2
WINTITGDPTYAQGFTG

SEQ ID NO: 21 - protein, 32 aa, synthetic construct
  REGION 1..32: note = MF3998 FR3
RFVFSLDTSVSTAYLQISSLKAEDTGVYYCAR

SEQ ID NO: 22 - protein, 11 aa, synthetic construct
  REGION 1..11: note = MF3998 CDR3
EEFLEWLFFDY

SEQ ID NO: 23 - protein, 11 aa, synthetic construct
  REGION 1..11: note = MF3998 FR4
WGQGTLVTVSS

SEQ ID NO: 24 - DNA, 369 bp, synthetic construct
  misc_feature 1..369: note = MF4010: heavy chain variable region sequence of an EGFR binding antibody:
  source 1..369: mol_type = other DNA
  CDS 1..369
caggtgcagctggtgcagtctgggtctgagttgaagaagcctggggcctcagtgaaggtttcctgcaaggcttctggatacaccttcactaacaatgccatgaattgggtgcgacaggcccctggacaagggcttgagtggatgggatggatcaacaccatcactggggacccatcgtatgcccagggcttcacaggacggtttgtcttctccctggacacctctgtcaacacggcatatctgcagatcagcagcctgaaggctgaggacactgccgtatattactgtgcgagagaggaatttttggagtggttattctttgactactggggccagggaaccctggtcaccgtctcaagcgtctccagt

SEQ ID NO: 25 - protein, 123 aa, synthetic construct
  REGION 1..123: note = Synthetic Construct
QVQLVQSGSELKKPGASVKVSCKASGYTFTNNAMNWVRQAPGQGLEWMGWINTITGDPSYAQGFTGRFVFSLDTSVNTAYLQISSLKAEDTAVYYCAREEFLEWLFFDYWGQGTLVTVSSVSS

SEQ ID NO: 26 - protein, 30 aa, synthetic construct; identical to SEQ ID NO: 17
  REGION 1..30: note = MF4010 FR1

SEQ ID NO: 27 - protein, 5 aa, synthetic construct
  REGION 1..5: note = MF40101 FR1
NNAMN

SEQ ID NO: 28 - protein, 14 aa, synthetic construct
  REGION 1..14: note = MF4010 FR2
WVRQAPGQGLEWMG

SEQ ID NO: 29 - protein, 17 aa, synthetic construct
  REGION 1..17: note = MF4010 CDR2
WINTITGDPSYAQGFTG

SEQ ID NO: 30 - protein, 32 aa, synthetic construct
  REGION 1..32: note = MF4010 FR3
RFVFSLDTSVNTAYLQISSLKAEDTAVYYCAR

SEQ ID NO: 31 - protein, 11 aa, synthetic construct
  REGION 1..11: note = MF40101 CDR3
EEFLEWLFFDY

SEQ ID NO: 32 - protein, 11 aa, synthetic construct
  REGION 1..11: note = MF4010 FR4
WGQGTLVTVSS

SEQ ID NO: 33 - DNA, 366 bp, synthetic construct
  misc_feature 1..366: note = MF4003: heavy chain variable region sequence of an EGFR binding antibody:
  source 1..366: mol_type = other DNA
  CDS 1..366
caggtgcagctggtgcaatctgggtctgagttgaagaagcctggggcctcagtgaaggtttcctgcaaggcttctggatacaccttccctagttttgctatgaattggcttcgacaggcccctggacaagggcttgagtggatgggatggatcaccaccaacactggggacccaacgtatgcccagggcttctcaggacggtttgtgttctccctggacacctctgtcagcacggcatatctgcagatcagcagcctaaaggctgaggacactgccgtgtattactgtgcgagagtttataactggataaggggatttgactactggggccagggaaccctggtcaccgtctcaagcgtctccagt

SEQ ID NO: 34 - protein, 122 aa, synthetic construct
  REGION 1..122: note = Synthetic Construct
QVQLVQSGSELKKPGASVKVSCKASGYTFPSFAMNWLRQAPGQGLEWMGWITTNTGDPTYAQGFSGRFVFSLDTSVSTAYLQISSLKAEDTAVYYCARVYNWIRGFDYWGQGTLVTVSSVSS

SEQ ID NO: 35 - protein, 30 aa, synthetic construct
  REGION 1..30: note = MF4003 FR1
QVQLVQSGSELKKPGASVKVSCKASGYTFP

SEQ ID NO: 36 - protein, 5 aa, synthetic construct
  REGION 1..5: note = MF4003 CDR1
SFAMN

SEQ ID NO: 37 - protein, 14 aa, synthetic construct
  REGION 1..14: note = MF4003 FR2
WLRQAPGQGLEWMG

SEQ ID NO: 38 - protein, 17 aa, synthetic construct
  REGION 1..17: note = MF4003 CDR2
WITTNTGDPTYAQGFSG

SEQ ID NO: 39 - protein, 32 aa, synthetic construct
  REGION 1..32: note = MF4003 FR3
RFVFSLDTSVSTAYLQISSLKAEDTAVYYCAR

SEQ ID NO: 40 - protein, 10 aa, synthetic construct
  REGION 1..10: note = MF4003 CDR3
VYNWIRGFDY

SEQ ID NO: 41 - protein, 11 aa, synthetic construct
  REGION 1..11: note = MF4003 FR4
WGQGTLVTVSS

SEQ ID NO: 42 - DNA, 385 bp, synthetic construct
  misc_feature 1..385: note = MF4289: heavy chain variable region sequence of an EGFR binding antibody:
  source 1..385: mol_type = other DNA
  CDS 2..385
ggcccagccggccatggcccaggtgcagctggtgcaatctgggtctgaattgaagaagcctggggcctcagtgaaggtttcctgcaagacttctggatacaccttcactgactatgctatgacttgggtgcgacaggcccctggacaagggcttgaatggatgggatggatcaccaccaacactggggacccaacgtatgccccgggcttcacaggacggtttgtcttctccttggacacctctgtcagcacggcatatctgcagatcagcagcctaaaggccgaggacactgccgtatattactgtgcgagagtgtatcattggatacggggatttgagttttggggccagggaaccctggtcaccgtctcaagcgtctccagt

SEQ ID NO: 43 - protein, 128 aa, synthetic construct
  REGION 1..128: note = Synthetic Construct
AQPAMAQVQLVQSGSELKKPGASVKVSCKTSGYTFTDYAMTWVRQAPGQGLEWMGWITTNTGDPTYAPGFTGRFVFSLDTSVSTAYLQISSLKAEDTAVYYCARVYHWIRGFEFWGQGTLVTVSSVSS

SEQ ID NO: 44 - protein, 119 aa, synthetic construct
  REGION 1..119: note = MF4289 complete VH sequence
QVQLVQSGSELKKPGASVKVSCKTSGYTFTDYAMTWVRQAPGQGLEWMGWITTNTGDPTYAPGFTGRFVFSLDTSVSTAYLQISSLKAEDTAVYYCARVYHWIRGFEFWGQGTLVTVSS

SEQ ID NO: 45 - protein, 30 aa, synthetic construct
  REGION 1..30: note = MF4289 FR1
QVQLVQSGSELKKPGASVKVSCKTSGYTFT

SEQ ID NO: 46 - protein, 5 aa, synthetic construct
  REGION 1..5: note = MF4289 CDR1
DYAMT

SEQ ID NO: 47 - protein, 14 aa, synthetic construct
  REGION 1..14: note = MF4289 FR2
WVRQAPGQGLEWMG

SEQ ID NO: 48 - protein, 17 aa, synthetic construct
  REGION 1..17: note = MF4289 CDR2
WITTNTGDPTYAPGFTG

SEQ ID NO: 49 - protein, 32 aa, synthetic construct; identical to SEQ ID NO: 39
  REGION 1..32: note = MF4289 FR3

SEQ ID NO: 50 - protein, 10 aa, synthetic construct
  REGION 1..10: note = MF4289 CDR3
VYHWIRGFEF

SEQ ID NO: 51 - protein, 11 aa, synthetic construct
  REGION 1..11: note = MF4289 FR4
WGQGTLVTVSS

SEQ ID NO: 52 - DNA, 366 bp, synthetic construct
  misc_feature 1..366: note = MF3370: heavy chain variable region sequence of an EGFR binding antibody:
  source 1..366: mol_type = other DNA
  CDS 1..366
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggctgtgtattactgtgcaaaagatcgtcattggcattggtggctggacgcctttgattattggggccaaggtaccctggtcaccgtctccagt

SEQ ID NO: 53 - protein, 122 aa, synthetic construct
  REGION 1..122: note = Synthetic Construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAKDRHWHWWLDAFDYWGQGTLVTVSS

SEQ ID NO: 54 - protein, 30 aa, synthetic construct
  REGION 1..30: note = MF3370 FR1
QVQLVQSGAEVKKPGASVKVSCKASGYTFT

SEQ ID NO: 55 - protein, 5 aa, synthetic construct
  REGION 1..5: note = MF3370 CDR1
SYGIS

SEQ ID NO: 56 - protein, 14 aa, synthetic construct
  REGION 1..14: note = MF3370 FR2
WVRQAPGQGLEWMG

SEQ ID NO: 57 - protein, 17 aa, synthetic construct
  REGION 1..17: note = MF3370 CDR2
WISAYNGNTNYAQKLQG

SEQ ID NO: 58 - protein, 32 aa, synthetic construct
  REGION 1..32: note = MF3370 FR3
RVTMTTDTSTSTAYMELRSLRSDDTAVYYCAK

SEQ ID NO: 59 - protein, 13 aa, synthetic construct
  REGION 1..13: note = MF3370 CDR3
DRHWHWWLDAFDY

SEQ ID NO: 60 - protein, 11 aa, synthetic construct
  REGION 1..11: note = MF3370 FR4
WGQGTLVTVSS

SEQ ID NO: 61 - DNA, 366 bp, synthetic construct
  misc_feature 1..366: note = MF4002: heavy chain variable region sequence of an EGFR binding antibody:
  source 1..366: mol_type = other DNA
  CDS 1..366
caggtgcagctggtgcaatctgggtctgagttgaagaagcctgggtcctcagtgaaggtttcctgcaaggcttctggatacaccttcactaactatgctatgaattgggtgcgacaggcccctggacaagggcttgagtggatgggatggatcaccaccaacactggggacccaacgtatgcccagggcttcacaggacgttttgtcttctccttggacacctctgtcagtacggcatatctgcagatcagcagcctaaaggctgaggacactgccgtatattactgtgtgagagtgtataactggataaggggatttgactactggggccagggaaccctggtcaccgtctcaagcgtctccagt

SEQ ID NO: 62 - protein, 122 aa, synthetic construct
  REGION 1..122: note = Synthetic Construct
QVQLVQSGSELKKPGSSVKVSCKASGYTFTNYAMNWVRQAPGQGLEWMGWITTNTGDPTYAQGFTGRFVFSLDTSVSTAYLQISSLKAEDTAVYYCVRVYNWIRGFDYWGQGTLVTVSSVSS

SEQ ID NO: 63 - protein, 30 aa, synthetic construct
  REGION 1..30: note = MF4002 FR1
QVQLVQSGSELKKPGSSVKVSCKASGYTFT

SEQ ID NO: 64 - protein, 5 aa, synthetic construct
  REGION 1..5: note = MF4002 CDR1
NYAMN

SEQ ID NO: 65 - protein, 14 aa, synthetic construct
  REGION 1..14: note = MF4002 FR2
WVRQAPGQGLEWMG

SEQ ID NO: 66 - protein, 17 aa, synthetic construct
  REGION 1..17: note = MF4002 CDR2
WITTNTGDPTYAQGFTG

SEQ ID NO: 67 - protein, 32 aa, synthetic construct
  REGION 1..32: note = MF4002 FR3
RFVFSLDTSVSTAYLQISSLKAEDTAVYYCVR

SEQ ID NO: 68 - protein, 10 aa, synthetic construct
  REGION 1..10: note = MF4002 CDR3
VYNWIRGFDY

SEQ ID NO: 69 - protein, 11 aa, synthetic construct
  REGION 1..11: note = MF4002 FR4
WGQGTLVTVSS

SEQ ID NO: 70 - DNA, 357 bp, synthetic construct
  misc_feature 1..357: note = MF3751: heavy chain variable region sequence of an EGFR binding antibody:
  source 1..357: mol_type = other DNA
  CDS 1..357
caggtgcagctggtacagtctggggctgaggtgaagaagcctggggcctcagtgaaggtttcctgcaaggcatctggatacaccttcaccggctactatatgcactgggtgcgacaggcccctggacaagggcttgagtggatgggaacaatcaaccctagtggtggtagcacatactacgcacagaagttccagggcagagtcaccatgaccagggacacgtccacgagcacagtctacatggagctgagcagcctgagatctgaggacacggccgtgtattactgtgcgagagatcggaactggggatgggactttgactactggggccagggaaccctggtcaccgtctccagt

SEQ ID NO: 71 - protein, 119 aa, synthetic construct
  REGION 1..119: note = Synthetic Construct
QVQLVQSGAEVKKPGASVKVSCKASGYTFTGYYMHWVRQAPGQGLEWMGTINPSGGSTYYAQKFQGRVTMTRDTSTSTVYMELSSLRSEDTAVYYCARDRNWGWDFDYWGQGTLVTVSS

SEQ ID NO: 72 - protein, 30 aa, synthetic construct; identical to SEQ ID NO: 54
  REGION 1..30: note = MF3751 FR1

SEQ ID NO: 73 - protein, 5 aa, synthetic construct
  REGION 1..5: note = MF3751 CDR1
GYYMH

SEQ ID NO: 74 - protein, 14 aa, synthetic construct
  REGION 1..14: note = MF3751 FR2
WVRQAPGQGLEWMG

SEQ ID NO: 75 - protein, 17 aa, synthetic construct
  REGION 1..17: note = MF3751 CDR2
TINPSGGSTYYAQKFQG

SEQ ID NO: 76 - protein, 32 aa, synthetic construct
  REGION 1..32: note = MF3751 FR3
RVTMTRDTSTSTVYMELSSLRSEDTAVYYCAR

SEQ ID NO: 77 - protein, 10 aa, synthetic construct
  REGION 1..10: note = MF3751 CDR3
DRNWGWDFDY

SEQ ID NO: 78 - protein, 11 aa, synthetic construct
  REGION 1..11: note = MF3751 FR4
WGQGTLVTVSS

SEQ ID NO: 79 - DNA, 366 bp, synthetic construct
  misc_feature 1..366: note = MF3752: heavy chain variable region sequence of an EGFR binding antibody:
  source 1..366: mol_type = other DNA
  CDS 1..366
gaggtgcagctggtggagtctgggcctgaggtgaagaagcctggggcctcagtgaaggtttcctgcaaggcatctggatacaccttcaccagctactatatgcactgggtgcgacaggcccctggacaagggcttgagtggatgggaacaatcaaccctagtggtggtagcacatactacgcacagaagttccagggcagagtcaccctgaccagggacacgtccacgagcacagtctacatggtgctgagcagcctgagatctgaggacacggccgtgtattactgtgcgagagatcggaactggggatgggactttgactactggggccagggaaccctggtcaccgtctcaagcgtctccagt

SEQ ID NO: 80 - protein, 122 aa, synthetic construct
  REGION 1..122: note = Synthetic Construct
EVQLVESGPEVKKPGASVKVSCKASGYTFTSYYMHWVRQAPGQGLEWMGTINPSGGSTYYAQKFQGRVTLTRDTSTSTVYMVLSSLRSEDTAVYYCARDRNWGWDFDYWGQGTLVTVSSVSS

SEQ ID NO: 81 - protein, 30 aa, synthetic construct
  REGION 1..30: note = MF3752 FR1
EVQLVESGPEVKKPGASVKVSCKASGYTFT

SEQ ID NO: 82 - protein, 5 aa, synthetic construct
  REGION 1..5: note = FR3752 CDR1
SYYMH

SEQ ID NO: 83 - protein, 14 aa, synthetic construct
  REGION 1..14: note = MF3752 FR2
WVRQAPGQGLEWMG

SEQ ID NO: 84 - protein, 17 aa, synthetic construct
  REGION 1..17: note = MF3752 CDR2
TINPSGGSTYYAQKFQG

SEQ ID NO: 85 - protein, 32 aa, synthetic construct
  REGION 1..32: note = MF3752 FR3
RVTLTRDTSTSTVYMVLSSLRSEDTAVYYCAR

SEQ ID NO: 86 - protein, 10 aa, synthetic construct
  REGION 1..10: note = MF3752 CDR3
DRNWGWDFDY

SEQ ID NO: 87 - protein, 11 aa, synthetic construct
  REGION 1..11: note = MF3752 FR4
WGQGTLVTVSS

SEQ ID NO: 88 - DNA, 391 bp, synthetic construct
  misc_feature 1..391: note = MF3178: heavy chain variable region sequence of an erbB-3 binding antibody
  source 1..391: mol_type = other DNA
  CDS 20..391
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcacgatgaccagggacacgtccatcagcacagcctacatggagctgagcaggctgagatctgacgacacggctgtgtattactgtgcaagagatcatggttctcgtcatttctggtcttactggggctttgattattggggccaaggtaccctggtcaccgtctccagt

SEQ ID NO: 89 - protein, 124 aa, synthetic construct
  REGION 1..124: note = Synthetic Construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDHGSRHFWSYWGFDYWGQGTLVTVSS

SEQ ID NO: 90 - protein, 5 aa, synthetic construct
  REGION 1..5: note = MF3178 CDR1
GYYMH

SEQ ID NO: 91 - protein, 17 aa, synthetic construct
  REGION 1..17: note = MF3178 CDR2
WINPNSGGTNYAQKFQG

SEQ ID NO: 92 - protein, 15 aa, synthetic construct
  REGION 1..15: note = MF3178 CDR3
DHGSRHFWSYWGFDY

SEQ ID NO: 93 - DNA, 385 bp, synthetic construct
  misc_feature 1..385: note = MF3176: heavy chain variable region sequence of an erbB-3 binding antibody
  source 1..385: mol_type = other DNA
  CDS 20..385
ggcccagccggccatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggctgtgtattactgtgcaagagattggtggtacccgccgtactactggggctttgattattggggccaaggtaccctggtcaccgtctccagt

SEQ ID NO: 94 - protein, 122 aa, synthetic construct
  REGION 1..122: note = Synthetic Construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDWWYPPYYWGFDYWGQGTLVTVSS

SEQ ID NO: 95 - protein, 5 aa, synthetic construct
  REGION 1..5: note = MF3176 CDR1
SYAMS

SEQ ID NO: 96 - protein, 17 aa, synthetic construct
  REGION 1..17: note = MF3176 CDR2
AISGSGGSTYYADSVKG

SEQ ID NO: 97 - protein, 13 aa, synthetic construct
  REGION 1..13: note = MF3176 CDR3
DWWYPPYYWGFDY

SEQ ID NO: 98 - DNA, 391 bp, synthetic construct
  misc_feature 1..391: note = MF3163: heavy chain variable region sequence of an erbB-3 binding antibody
  source 1..391: mol_type = other DNA
  CDS 20..391
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcacgatgaccagggacacgtccatcagcacagcctacatggagctgagcaggctgagatctgacgacacggccgtgtattactgtgcaaaagattcttactctcgtcatttctactcttggtgggcctttgattattggggccaaggtaccctggtcaccgtctccagt

SEQ ID NO: 99 - protein, 124 aa, synthetic construct
  REGION 1..124: note = Synthetic Construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAKDSYSRHFYSWWAFDYWGQGTLVTVSS

SEQ ID NO: 100 - protein, 5 aa, synthetic construct
  REGION 1..5: note = MF3163 CDR1
GYYMH

SEQ ID NO: 101 - protein, 17 aa, synthetic construct
  REGION 1..17: note = MF3163 CDR2
WINPNSGGTNYAQKFQG

SEQ ID NO: 102 - protein, 15 aa, synthetic construct
  REGION 1..15: note = MF3163 CDR3
DSYSRHFYSWWAFDY

SEQ ID NO: 103 - DNA, 382 bp, synthetic construct
  misc_feature 1..382: note = MF3099: heavy chain variable region sequence of an erbB-3 binding antibody
  source 1..382: mol_type = other DNA
  CDS 20..382
ggcccagccggccatggccgaggtccagctgcagcagcctggggctgagctggtgaggcctgggacttcagtgaagttgtcctgcaaggcttctggctacaccttcaccagctactggatgcactgggtaaagcagaggcctggacaaggccttgagtggatcggaattcttgatccttctgatagttatactacctacaatcaaaagttcaagggcaaggccacattaacagtagacacatcctccagcatagcctacatgcagctcagcagcctgacatctgaggactctgcgctctattactgtgcaagagggggagattacgacgagggaggtgctatggactactggggtcaaggaacctcggtcaccgtctccagt

SEQ ID NO: 104 - protein, 121 aa, synthetic construct
  REGION 1..121: note = Synthetic Construct
EVQLQQPGAELVRPGTSVKLSCKASGYTFTSYWMHWVKQRPGQGLEWIGILDPSDSYTTYNQKFKGKATLTVDTSSSIAYMQLSSLTSEDSALYYCARGGDYDEGGAMDYWGQGTSVTVSS

SEQ ID NO: 105 - protein, 5 aa, synthetic construct
  REGION 1..5: note = MF3099 CDR1
SYWMH

SEQ ID NO: 106 - protein, 17 aa, synthetic construct
  REGION 1..17: note = MF3099 CDR2
ILDPSDSYTTYNQKFKG

SEQ ID NO: 107 - protein, 12 aa, synthetic construct
  REGION 1..12: note = MF3099 CDR3
GGDYDEGGAMDY

SEQ ID NO: 108 - DNA, 391 bp, synthetic construct
  misc_feature 1..391: note = MF3307: heavy chain variable region sequence of an erbB-3 binding antibody
  source 1..391: mol_type = other DNA
  CDS 20..391
ggcccagccggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcacgatgaccagggacacgtccatcagcacagcctacatggagctgagcaggctgagatctgacgacacggccgtgtattactgtgcaagaggttctcgtaaacgtctgtctaactacttcaacgcctttgattattggggccaaggtaccctggtcaccgtctccagt

SEQ ID NO: 109 - protein, 124 aa, synthetic construct
  REGION 1..124: note = Synthetic Construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARGSRKRLSNYFNAFDYWGQGTLVTVSS

SEQ ID NO: 110 - protein, 5 aa, synthetic construct
  REGION 1..5: note = MF3307 CDR1
GYYMH

SEQ ID NO: 111 - protein, 17 aa, synthetic construct
  REGION 1..17: note = MF3307 CDR2
WINPNSGGTNYAQKFQG

SEQ ID NO: 112 - protein, 15 aa, synthetic construct
  REGION 1..15: note = MF3307 CDR3
GSRKRLSNYFNAFDY

SEQ ID NO: 113 - DNA, 372 bp, synthetic construct
  misc_feature 1..372: note = MF6055_VH
  source 1..372: mol_type = other DNA
  CDS 1..372
caggtgcagctggtgcagtctggggctgacgtgaagaagcctggggcctcagtgaaggtctcctgcaaggcttctggatacaccttcaccggctactatatgcactgggtgcgacaggcccctggacaagctcttgagtggatgggatggatcaacccttctagtggtggcacaaactatgcaaagaagtttcagggcagggtcacgatgaccagggagacgtccacaagcacagcctacatggagctgagcaggctgagatctgacgacacggctacgtattactgtgcaagagatcatggttctcgtcatttctggtcttactggggctttgattattggggccaaggtaccctggtcaccgtctccagt

SEQ ID NO: 114 - protein, 124 aa, synthetic construct
  REGION 1..124: note = Synthetic Construct
QVQLVQSGADVKKPGASVKVSCKASGYTFTGYYMHWVRQAPGQALEWMGWINPSSGGTNYAKKFQGRVTMTRETSTSTAYMELSRLRSDDTATYYCARDHGSRHFWSYWGFDYWGQGTLVTVSS

SEQ ID NO: 115 - DNA, 372 bp, synthetic construct
  misc_feature 1..372: note = MF6056_VH
  source 1..372: mol_type = other DNA
  CDS 1..372
caggtgcagctggtgcagtctggggctgacgtgaagaagcctggggcctcagtgaaggtcacgtgcaaggcttctggatacaccttcaccggctactatatgcactgggtgcgacaggcccctggacaagctcttgagtggatgggatggatcaacccttctagtggtggcacaaactatgcaaagaagtttcagggcagggtctctatgaccagggagacgtccacaagcacagcctacatgcagctgagcaggctgagatctgacgacacggctacgtattactgtgcaagagatcatggttctcgtcatttctggtcttactggggctttgattattggggccaaggtaccctggtcaccgtctccagt

SEQ ID NO: 116 - protein, 124 aa, synthetic construct
  REGION 1..124: note = Synthetic Construct
QVQLVQSGADVKKPGASVKVTCKASGYTFTGYYMHWVRQAPGQALEWMGWINPSSGGTNYAKKFQGRVSMTRETSTSTAYMQLSRLRSDDTATYYCARDHGSRHFWSYWGFDYWGQGTLVTVSS

SEQ ID NO: 117 - DNA, 372 bp, synthetic construct
  misc_feature 1..372: note = MF6057_VH
  source 1..372: mol_type = other DNA
  CDS 1..372
caggtgcagctggtgcagtctggggctgatgtgaagaagcctggggcctcagtgaaggtcacgtgcaaggcttctggatacaccttcaccggctactatatgcactgggtgcgacaggcccctggacaagggcttgagtggatgggatggatcaaccctcagagtggtggcacaaactatgcacagaagtttcagggcagggtcacgatgaccagggacacgtccatcagcacagcctacatgcagctgagcaggctgagatctgacgacacggctgtgtattactgtgcaagagatcatggttctcgtcatttctggtcttactggggctttgattattggggccaaggtaccctggtcaccgtctccagt

SEQ ID NO: 118 - protein, 124 aa, synthetic construct
  REGION 1..124: note = Synthetic Construct
QVQLVQSGADVKKPGASVKVTCKASGYTFTGYYMHWVRQAPGQGLEWMGWINPQSGGTNYAQKFQGRVTMTRDTSISTAYMQLSRLRSDDTAVYYCARDHGSRHFWSYWGFDYWGQGTLVTVSS

SEQ ID NO: 119 - DNA, 372 bp, synthetic construct
  misc_feature 1..372: note = MF6058_VH
  source 1..372: mol_type = other DNA
  CDS 1..372
caggtgcagctggtgcagtctggggctgacgtgaagaagcctggggcctcagtgaaggtcacgtgcaaggcttctggatacaccttcaccggctactatatgcactgggtgcgacaggcccctggacaagctcttgagtggatgggatggatcaaccctcaaagtggtggcacaaactatgcaaagaagtttcagggcagggtctctatgaccagggagacgtccacaagcacagcctacatgcagctgagcaggctgagatctgacgacacggctacgtattactgtgcaagagatcatggttctcgtcatttctggtcttactggggctttgattattggggccaaggtaccctggtcaccgtctccagt

SEQ ID NO: 120 - protein, 124 aa, synthetic construct
  REGION 1..124: note = Synthetic Construct
QVQLVQSGADVKKPGASVKVTCKASGYTFTGYYMHWVRQAPGQALEWMGWINPQSGGTNYAKKFQGRVSMTRETSTSTAYMQLSRLRSDDTATYYCARDHGSRHFWSYWGFDYWGQGTLVTVSS

SEQ ID NO: 121 - DNA, 372 bp, synthetic construct
  misc_feature 1..372: note = MF6059_VH
  source 1..372: mol_type = other DNA
  CDS 1..372
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctggcagtggttctacaaactatgcacagaagtttcagggcagggtcacgatgaccagggacacgtccatcagcacagcctacatggagctgagcaggctgagatctgacgacacggctgtgtattactgtgcaagagatcatggttctcgtcatttctggtcttactggggctttgattattggggccaaggtaccctggtcaccgtctccagt

SEQ ID NO: 122 - protein, 124 aa, synthetic construct
  REGION 1..124: note = Synthetic Construct
QVQLVQSGAEVKKPGASVKVSCKASGYTFTGYYMHWVRQAPGQGLEWMGWINPGSGSTNYAQKFQGRVTMTRDTSISTAYMELSRLRSDDTAVYYCARDHGSRHFWSYWGFDYWGQGTLVTVSS

SEQ ID NO: 123 - DNA, 372 bp, synthetic construct
  misc_feature 1..372: note = MF6060_VH
  source 1..372: mol_type = other DNA
  CDS 1..372
caggtgcagctggtgcagtctggggctgacgtgaagaagcctggggcctcagtgaaggtctcctgcaaggcttctggatacaccttcaccggctactatatgcactgggtgcgacaggcccctggacaagctcttgagtggatgggatggatcaaccctcaaagtggtggcacaaactatgcaaagaagtttcagggcagggtcacgatgaccagggagacgtccacaagcacagcctacatggagctgagcaggctgagatctgacgacacggctacgtattactgtgcaagagatcatggttctcgtcatttctggtcttactggggctttgattattggggccaaggtaccctggtcaccgtctccagt

SEQ ID NO: 124 - protein, 124 aa, synthetic construct
  REGION 1..124: note = Synthetic Construct
QVQLVQSGADVKKPGASVKVSCKASGYTFTGYYMHWVRQAPGQALEWMGWINPQSGGTNYAKKFQGRVTMTRETSTSTAYMELSRLRSDDTATYYCARDHGSRHFWSYWGFDYWGQGTLVTVSS

SEQ ID NO: 125 - DNA, 372 bp, synthetic construct
  misc_feature 1..372: note = MF6061_VH
  source 1..372: mol_type = other DNA
  CDS 1..372
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctcagagtggtggcacaaactatgcacagaagtttaagggcagggtcacgatgaccagggacacgtccaccagcacagcctacatggagctgagcaggctgagatctgacgacacggctgtgtattactgtgcaagagatcatggttctcgtcatttctggtcttactggggctttgattattggggccaaggtaccctggtcaccgtctccagt

SEQ ID NO: 126 - protein, 124 aa, synthetic construct
  REGION 1..124: note = Synthetic Construct
QVQLVQSGAEVKKPGASVKVSCKASGYTFTGYYMHWVRQAPGQGLEWMGWINPQSGGTNYAQKFKGRVTMTRDTSTSTAYMELSRLRSDDTAVYYCARDHGSRHFWSYWGFDYWGQGTLVTVSS

SEQ ID NO: 127 - DNA, 372 bp, synthetic construct
  misc_feature 1..372: note = MF6062_VH
  source 1..372: mol_type = other DNA
  CDS 1..372
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctggcagtggttctacaaactatgcacagaagtttcagggcagggtcacgatgaccagggacacgtccacaagcacagcctacatggagctgagcaggctgagatctgacgacacggctgtgtattactgtgcaagagatcatggttctcgtcatttctggtcttactggggctttgattattggggccaaggtaccctggtcaccgtctccagt

SEQ ID NO: 128 - protein, 124 aa, synthetic construct
  REGION 1..124: note = Synthetic Construct
QVQLVQSGAEVKKPGASVKVSCKASGYTFTGYYMHWVRQAPGQGLEWMGWINPGSGSTNYAQKFQGRVTMTRDTSTSTAYMELSRLRSDDTAVYYCARDHGSRHFWSYWGFDYWGQGTLVTVSS

SEQ ID NO: 129 - DNA, 372 bp, synthetic construct
  misc_feature 1..372: note = MF6063_VH
  source 1..372: mol_type = other DNA
  CDS 1..372
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctcagagtggtggcacaaactatgcaaagaagtttcagggcagggtcacgatgaccagggacacgtccaccagcacagcctacatggagctgagcaggctgagatctgacgacacggctgtgtattactgtgcaagagatcatggttctcgtcatttctggtcttactggggctttgattattggggccaaggtaccctggtcaccgtctccagt

SEQ ID NO: 130 - protein, 124 aa, synthetic construct
  REGION 1..124: note = Synthetic Construct
QVQLVQSGAEVKKPGASVKVSCKASGYTFTGYYMHWVRQAPGQGLEWMGWINPQSGGTNYAKKFQGRVTMTRDTSTSTAYMELSRLRSDDTAVYYCARDHGSRHFWSYWGFDYWGQGTLVTVSS

SEQ ID NO: 131 - DNA, 372 bp, synthetic construct
  misc_feature 1..372: note = MF6064_VH
  source 1..372: mol_type = other DNA
  CDS 1..372
caggtgcagctggtgcagtctggggctgaggtgaagaagcctggggcctcagtgaaggtctcctgcaaggcttctggatacaccttcaccggctactatatgcactgggtgcgacaggcccctggaaaggggcttgagtggatgggatggatcaaccctcagagtggtggcacaaactatgcacagaagtttcagggcagggtcacgatgaccagggacacgtccacgagcacagcctacatggagctgagcaggctgagatctgacgacacggctgtgtattactgtgcaagagatcatggttctcgtcatttctggtcttactggggctttgattattggggccaaggtaccctggtcaccgtctccagt

SEQ ID NO: 132 - protein, 124 aa, synthetic construct
  REGION 1..124: note = Synthetic Construct
QVQLVQSGAEVKKPGASVKVSCKASGYTFTGYYMHWVRQAPGKGLEWMGWINPQSGGTNYAQKFQGRVTMTRDTSTSTAYMELSRLRSDDTAVYYCARDHGSRHFWSYWGFDYWGQGTLVTVSS

SEQ ID NO: 133 - DNA, 372 bp, synthetic construct
  misc_feature 1..372: note = MF6065_VH
  source 1..372: mol_type = other DNA
  CDS 1..372
caggtgcagctggtgcagtctggggctgaggtgaagaagcctggggcctcagtgaaggtctcctgcaaggcttctggatacaccttcacctcttactatatgcactgggtgcgacaggcccctggacaagggcttgagtggatgggatggatcaaccctcaggggggttctacaaactatgcacagaagtttcagggcagggtcacgatgaccagggacacgtccaccagcacagtgtacatggagctgagcaggctgagatctgaggacacggctgtgtattactgtgcaagagatcatggttctcgtcatttctggtcttactggggctttgattattggggccaaggtaccctggtcaccgtctccagt

SEQ ID NO: 134 - protein, 124 aa, synthetic construct
  REGION 1..124: note = Synthetic Construct
QVQLVQSGAEVKKPGASVKVSCKASGYTFTSYYMHWVRQAPGQGLEWMGWINPQGGSTNYAQKFQGRVTMTRDTSTSTVYMELSRLRSEDTAVYYCARDHGSRHFWSYWGFDYWGQGTLVTVSS

SEQ ID NO: 135 - DNA, 372 bp, synthetic construct
  misc_feature 1..372: note = MF6066_VH
  source 1..372: mol_type = other DNA
  CDS 1..372
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctcagagtggttctacaaactatgcacagaagtttcagggcagggtcacgatgaccagggacacgtccaccagcacagcctacatggagctgagctctctgagatctgaggacacggctgtgtattactgtgcaagagatcatggttctcgtcatttctggtcttactggggctttgattattggggccaaggtaccctggtcaccgtctccagt

SEQ ID NO: 136 - protein, 124 aa, synthetic construct
  REGION 1..124: note = Synthetic Construct
QVQLVQSGAEVKKPGASVKVSCKASGYTFTGYYMHWVRQAPGQGLEWMGWINPQSGSTNYAQKFQGRVTMTRDTSTSTAYMELSSLRSEDTAVYYCARDHGSRHFWSYWGFDYWGQGTLVTVSS

SEQ ID NO: 137 - DNA, 372 bp, synthetic construct
  misc_feature 1..372: note = MF6067_VH
  source 1..372: mol_type = other DNA
  CDS 1..372
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctcagagtggtggcacaaactatgcacagaagtttcagggcagggtcacgatgaccagggacacgtccaccagcacagtctacatggagctgagctctctgagatctgacgacacggctgtgtattactgtgcaagagatcatggttctcgtcatttctggtcttactggggctttgattattggggccaaggtaccctggtcaccgtctccagt

SEQ ID NO: 138 - protein, 124 aa, synthetic construct
  REGION 1..124: note = Synthetic Construct
QVQLVQSGAEVKKPGASVKVSCKASGYTFTGYYMHWVRQAPGQGLEWMGWINPQSGGTNYAQKFQGRVTMTRDTSTSTVYMELSSLRSDDTAVYYCARDHGSRHFWSYWGFDYWGQGTLVTVSS

SEQ ID NO: 139 - DNA, 372 bp, synthetic construct
  misc_feature 1..372: note = MF6068_VH
  source 1..372: mol_type = other DNA
  CDS 1..372
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctcagagtggtggcacaaactatgcacagaagtttcagggcagggtcacgatgaccagggacacgtccaccagcacagcctacatggagctgagcaggctgagatctgacgacacggctgtgtattactgtgcaagagatcatggttctcgtcatttctggtcttactggggctttgattattggggccaaggtaccctggtcaccgtctccagt

SEQ ID NO: 140 - protein, 124 aa, synthetic construct
  REGION 1..124: note = Synthetic Construct
QVQLVQSGAEVKKPGASVKVSCKASGYTFTGYYMHWVRQAPGQGLEWMGWINPQSGGTNYAQKFQGRVTMTRDTSTSTAYMELSRLRSDDTAVYYCARDHGSRHFWSYWGFDYWGQGTLVTVSS

SEQ ID NO: 141 - DNA, 372 bp, synthetic construct
  misc_feature 1..372: note = MF6069_VH
  source 1..372: mol_type = other DNA
  CDS 1..372
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctcagagtggtggcacaaactatgcacagaagtttcagggcagggtcacgatgaccagggacacgtccatcagcacagcctacatggagctgagcaggctgagatctgacgacacggctgtgtattactgtgcaagagatcatggttctcgtcatttctggtcttactggggctttgattattggggccaaggtaccctggtcaccgtctccagt

SEQ ID NO: 142 - protein, 124 aa, synthetic construct
  REGION 1..124: note = Synthetic Construct
QVQLVQSGAEVKKPGASVKVSCKASGYTFTGYYMHWVRQAPGQGLEWMGWINPQSGGTNYAQKFQGRVTMTRDTSISTAYMELSRLRSDDTAVYYCARDHGSRHFWSYWGFDYWGQGTLVTVSS

SEQ ID NO: 143 - DNA, 372 bp, synthetic construct
  misc_feature 1..372: note = MF6070_VH
  source 1..372: mol_type = other DNA
  CDS 1..372
caggtgcagctggtgcagtctggggctgaggtgaagaagcctggggcctcagtgaaggtctcctgcaaggcttctggatacaccttcacctcttactatatgcactgggtgcgacaggcccctggacaagggcttgagtggatgggatggatcaacccttctgggggttctacaaactatgcacagaagtttcagggcagggtcacgatgaccagggacacgtccaccagcacagtgtacatggagctgagcaggctgagatctgaggacacggctgtgtattactgtgcaagagatcatggttctcgtcatttctggtcttactggggctttgattattggggccaaggtaccctggtcaccgtctccagt

SEQ ID NO: 144 - protein, 124 aa, synthetic construct
  REGION 1..124: note = Synthetic Construct
QVQLVQSGAEVKKPGASVKVSCKASGYTFTSYYMHWVRQAPGQGLEWMGWINPSGGSTNYAQKFQGRVTMTRDTSTSTVYMELSRLRSEDTAVYYCARDHGSRHFWSYWGFDYWGQGTLVTVSS

SEQ ID NO: 145 - DNA, 372 bp, synthetic construct
  misc_feature 1..372: note = MF6071_VH
  source 1..372: mol_type = other DNA
  CDS 1..372
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaacccttctagtggttctacaaactatgcacagaagtttcagggcagggtcacgatgaccagggacacgtccaccagcacagcctacatggagctgagctctctgagatctgaggacacggctgtgtattactgtgcaagagatcatggttctcgtcatttctggtcttactggggctttgattattggggccaaggtaccctggtcaccgtctccagt

SEQ ID NO: 146 - protein, 124 aa, synthetic construct
  REGION 1..124: note = Synthetic Construct
QVQLVQSGAEVKKPGASVKVSCKASGYTFTGYYMHWVRQAPGQGLEWMGWINPSSGSTNYAQKFQGRVTMTRDTSTSTAYMELSSLRSEDTAVYYCARDHGSRHFWSYWGFDYWGQGTLVTVSS

SEQ ID NO: 147 - DNA, 372 bp, synthetic construct
  misc_feature 1..372: note = MF6072_VH
  source 1..372: mol_type = other DNA
  CDS 1..372
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaacccttctagtggtggcacaaactatgcacagaagtttcagggcagggtcacgatgaccagggacacgtccaccagcacagtctacatggagctgagctctctgagatctgacgacacggctgtgtattactgtgcaagagatcatggttctcgtcatttctggtcttactggggctttgattattggggccaaggtaccctggtcaccgtctccagt

SEQ ID NO: 148 - protein, 124 aa, synthetic construct
  REGION 1..124: note = Synthetic Construct
QVQLVQSGAEVKKPGASVKVSCKASGYTFTGYYMHWVRQAPGQGLEWMGWINPSSGGTNYAQKFQGRVTMTRDTSTSTVYMELSSLRSDDTAVYYCARDHGSRHFWSYWGFDYWGQGTLVTVSS

SEQ ID NO: 149 - DNA, 372 bp, synthetic construct
  misc_feature 1..372: note = MF6073_VH
  source 1..372: mol_type = other DNA
  CDS 1..372
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaacccttctagtggtggcacaaactatgcacagaagtttcagggcagggtcacgatgaccagggacacgtccaccagcacagcctacatggagctgagcaggctgagatctgacgacacggctgtgtattactgtgcaagagatcatggttctcgtcatttctggtcttactggggctttgattattggggccaaggtaccctggtcaccgtctccagt

SEQ ID NO: 150 - protein, 124 aa, synthetic construct
  REGION 1..124: note = Synthetic Construct
QVQLVQSGAEVKKPGASVKVSCKASGYTFTGYYMHWVRQAPGQGLEWMGWINPSSGGTNYAQKFQGRVTMTRDTSTSTAYMELSRLRSDDTAVYYCARDHGSRHFWSYWGFDYWGQGTLVTVSS

SEQ ID NO: 151 - DNA, 372 bp, synthetic construct
  misc_feature 1..372: note = MF6074_VH
  source 1..372: mol_type = other DNA
  CDS 1..372
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaacccttctagtggtggcacaaactatgcacagaagtttcagggcagggtcacgatgaccagggacacgtccatcagcacagcctacatggagctgagcaggctgagatctgacgacacggctgtgtattactgtgcaagagatcatggttctcgtcatttctggtcttactggggctttgattattggggccaaggtaccctggtcaccgtctccagt

SEQ ID NO: 152 - protein, 124 aa, synthetic construct
  REGION 1..124: note = Synthetic Construct
QVQLVQSGAEVKKPGASVKVSCKASGYTFTGYYMHWVRQAPGQGLEWMGWINPSSGGTNYAQKFQGRVTMTRDTSISTAYMELSRLRSDDTAVYYCARDHGSRHFWSYWGFDYWGQGTLVTVSS

SEQ ID NO: 153 - protein, 214 aa, synthetic construct
  REGION 1..214: note = Common Light Chain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - protein, 454 aa, synthetic construct
  REGION 1..454: note = heavy chain for EGFR binding
QVQLVQSGAEVKKPGASVKVSCKVSGYTLTELSMHWVRQAPGKGLEWMGGFDPEYGKTFFAQNFQGRVTMTEDTSADTAYMELSSLRSEDTAVYYCATEGYYETTTYYYNLFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPG

SEQ ID NO: 155 - protein, 453 aa, synthetic construct
  REGION 1..453: note = heavy chain for erbB-3 binding
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDHGSRHFWSYWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPG

SEQ ID NO: 156 - protein, 17 aa, synthetic construct
  REGION 1..17: note = MF6058 CDR2
WINPQSGGTNYAKKFQG

SEQ ID NO: 157 - protein, 6 aa, synthetic construct
  REGION 1..6: note = Common Light Chain CDR1
QSISSY

SEQ ID NO: 158 - protein, 9 aa, synthetic construct
  REGION 1..9: note = Common Light Chain CDR3
QQSYSTPPT

(19) United States
(12) Patent Application Publication
(10) Pub. No.: US 2024/0052043 A1
(43) Pub. Date: Feb. 15, 2024

(54) ANTIBODIES THAT BIND EGFR AND ERBB3

(71) Applicant: Merus N.V., Utrecht (NL)

(72) Inventors: Ton LOGTENBERG, Utrecht (NL); Mark THROSBY, Utrecht (NL); Robertus Cornelis ROOVERS, Utrecht (NL)

(21) Appl. No.: 18/479,619

(22) Filed: Oct. 2, 2023

Related U.S. Application Data
(63) Continuation of application No. 17/076,143, filed on Oct. 21, 2020, now Pat. No. 11,820,825.
Application No. 17/076,143 is a division of application No. 15/121,619, filed on Aug. 25, 2016, now Pat. No. 10,844,127.

(30) Foreign Application Priority Data
Feb. 28, 2014 (EP) 14157351.9

(51) Int. Cl. C07K 16/28; C07K 16/32

(52) U.S. Cl. CPC C07K 16/2863; C07K 16/32; C07K 2317/31; C07K 2317/92; C07K 2317/565; C07K 2317/732; C07K 2317/76

(57) ABSTRACT

The invention relates in one aspect to bispecific antibodies comprising a first antigen-binding site that binds EGFR and a second antigen-binding site that binds Erb B-3, wherein the antibody has a half maximal growth inhibitory concentration (IC50) of less than 200 pM for inhibiting EGFR and/or Erb B-3 ligand induced growth of Bx PC3 cells or Bx PC3-luc2 cells. Further described are method for producing the bispecific antibodies and means and methods for the treatment of subjects with the antibodies.

### CROSS-REFERENCE TO RELATED APPLICATIONS

This application is a Divisional of U.S. application Ser. No. 17/076,143, filed Oct. 21, 2020, which is a Divisional of U.S. application Ser. No. 15/121,619, 371(c) Date Aug. 25, 2016, now U.S. Pat. No. 10,844,127, which is a 35 U.S.C. § 371 National Phase Application of PCT/NL2015/050124, filed Feb. 27, 2015, which claims priority to EP Application No. 14157351.9, filed Feb. 28, 2014.

### REFERENCE TO SEQUENCE LISTING SUBMITTED ELECTRONICALLY

The content of the electronically submitted sequence listing (Name: “4096_0090003_Seqlisting_ST25.txt”; Size: 184,951 bytes; and Date of Creation: Apr. 28, 2023) is herein incorporated by reference in its entirety.

The invention relates to the field of antibodies. In particular it relates to the field of therapeutic (human) antibodies for the treatment of diseases involving aberrant cells. More in particular it relates to antibodies that bind EGFR and ErbB-3 and their use in the binding of EGFR and ErbB-3 positive cells, particularly tumor cells.

The epidermal growth factor (EGF) receptor (EGFR) is the prototype cell-surface receptor for members of the epidermal growth factor family (EGF-family) of extracellular protein ligands. EGFR is also known as the ErbB-1 receptor. The receptor has been given various names in the past (EGFR; ERBB; ERBB1; HER1; PIG61; mENA). In the present invention the names ErbB-1, EGFR or HER1 in humans will be used interchangeably. EGFR is a member of the ErbB family of receptors, a subfamily of four closely related receptor tyrosine kinases: ErbB-1 (EGFR), ErbB-2 (HER2/c-neu; Her2), ErbB-3 (Her 3) and ErbB-4 (Her 4).

EGFR exists on the cell surface and is activated by binding of its specific ligands, including epidermal growth factor and transforming growth factor a (TGFα). Upon activation by its growth factor ligands, the receptor undergoes a transition from an inactive mostly monomeric form to an active homo-dimer. In addition to forming homo-dimers after ligand binding, EGFR may pair with another member of the ErbB receptor family, such as ErbB2, to create an activated hetero-dimer. There is also evidence to suggest that dimers form in the absence of ligand-binding and clusters of activated EGFRs form after ligand binding.

EGFR dimerization stimulates its intrinsic intracellular protein-tyrosine kinase (PTK) activity. This activity induces several signal transduction cascades that lead to cell proliferation and differentiation. The kinase domain of EGFR can cross-phosphorylate tyrosine residues of other receptors it is complexed with, and can itself be activated in that manner.

Mutations involving EGFR have been identified in several types of cancer, and it is the target of an expanding class of anticancer therapies. These include EGFR targeted small molecules as gefitinib and erlotinib for lung cancer, and antibodies as cetuximab and panitumab for colon cancer and head and neck cancer.

Cetuximab and panitumumab are monoclonal antibodies that inhibit the receptor. Other monoclonals in clinical development are zalutumumab, nimotuzumab, and matuzumab. The monoclonal antibodies aim to block the extracellular ligand-induced receptor activation, mostly by blocking ligand binding to the receptor. With the binding site blocked, signal-inducing molecules cannot attach effectively and thereby also not activate downstream signaling. However, ligand-induced receptor activation may also be inhibited by stabilization of the inactive receptor conformation (matuzumab).

Although there is some success with the EGFR targeted antibody therapy, most are associated with the development of treatment resistance over time. One of the ways in which EGFR positive tumors can escape the targeted therapy is by signaling through another receptor(dimer). For instance, increased signaling by EGFR/ErbB-3 (HER1/HER3) dimers due to increased HER3 expression or heregulin expression is associated with EGFR related drug resistance in lung cancers and head and neck cancers. Apart from the induction of treatment resistance, some side effects of EGFR-targeting antibodies have been observed. One example is the development of a skin rash, associated with efficient EGFR inhibition. When extreme, such rashes can lead to a reduction in treatment cycles and/or premature termination of treatment.

ErbB-3 does not have inherent kinase inactivity. Therefore, effective inhibition of ErbB-3 receptor signaling cannot be achieved with small molecule tyrosine kinase inhibitors (TKI's). Recently a monoclonal antibody termed MEHD7945A was found to show promise in a pre-clinical setting of EGFR positive tumors. MEHD7945A is a monoclonal antibody with two identical antigen-binding sites. MEHD7945A has the unique property that it has two identical antigen-binding arms that each individually have the capacity to bind either EGFR or ErbB-3, but not to other receptors. Once an antigen is bound, the antigen-binding site is blocked for the other antigen. Therefore, MEHD7945A (called a ‘two in one’ antibody) can be regarded both as an EGFR-targeting antibody, as well as a HER3 targeting antibody.

### SUMMARY OF THE INVENTION

The invention provides a bispecific antibody comprising a first antigen-binding site that binds EGFR and a second antigen-binding site that binds ErbB-3 and wherein the antibody has a half maximal growth inhibitory concentration (IC50) of less than 200 pM for inhibiting EGFR and/or ErbB-3 ligand induced growth of BxPC3 cells (ATCC CRL-1687) or BxPC3-luc2 cells (Perkin Elmer 125058).

Further provided is an antibody that comprises an antigen-binding site that binds EGFR, wherein the antibody comprises an immunoglobulin heavy chain with a heavy chain variable region that binds EGFR and that comprises the amino acid sequence of VH chain MF3998; MF4280; MF4002; MF4003; MF4010; MF4289; MF3370; MF3751; MF3752 as depicted in FIG. 11A, preferably wherein the immunoglobulin light chain variable region comprises the amino acid sequence of FIG. 11C

The invention further provides an antibody that comprises an antigen-binding site that binds ErbB-3, comprising an immunoglobulin heavy chain with a heavy chain variable region that binds ErbB-3 and that comprises the amino acid sequence of VH chain MF3178; MF3176; MF3163; MF3307, MF6055-MF6074, preferably MF3178, MF3176, MF3163, MF6058, MF6061 or MF6065 as depicted in FIG. 11B, preferably wherein the immunoglobulin light chain variable region comprises the amino acid sequence of FIG. 11C.

An antibody of the invention is, unless otherwise specifically specified, preferably a bispecific antibody.

The invention further provides a pharmaceutical composition comprising an antibody according to the invention.

Also provided is an antibody of the invention that further comprises a label, preferably a label for in vivo imaging.

The invention further provides a method for the treatment of a subject having a EGFR, ErB-3 or ECFR/ErbB-3 positive tumor or at risk of having said tumor comprising administering to the subject a bispecific antibody according to the invention. Also provided is a bispecific antibody according to the invention for use in the treatment of a subject having or at risk of having an EGFR. ErbB-3 or EGFR/ErbB-3 positive tumor.

### DETAILED DESCRIPTION OF THE INVENTION

As used herein, the term “antigen-binding site” refers to a site on an antibody which is capable of binding to antigen. The unmodified antigen-binding site is typically formed by and present in the variable domain of the antibody. In one embodiment an antibody variable domain of the invention comprises a heavy chain variable region (VH) and a light chain variable region (VL). The antigen-binding site can be present in the combined VH/VL variable domain, or in only the VH region or only the VL region. When the antigen-binding site is present in only one of the two regions of the variable domain, the counterpart variable region can contribute to the folding and/or stability of the binding variable region, but does not significantly contribute to the binding of the antigen itself.

Antigen binding by an antibody is typically mediated through the complementarity determining regions (CDR's) of the antibody and the specific three-dimensional structure of both the antigen and the variable domain allowing those two structures to bind together with precision (an interaction similar to a lock and key), as opposed to random, non-specific sticking of antibodies. As an antibody typically recognizes only part of an antigen (called the epitope), and as such epitope may be present in other, but preferably non-human, compounds as well, antibodies according to the present invention that bind EGFR and/or ErbB-3 may recognize other proteins as well, but preferably not other human proteins, if such other compounds contain the same epitope. Hence, the term “binding” does not exclude binding of the antibodies to another protein or protein(s) that contain the same epitope. Such other protein(s) is preferably not a human protein. Instead, cross-reactivity is allowed. An EGFR antigen-binding site and an ErbB-3 antigen-binding site as defined in the present invention typically do not bind to other proteins on the membrane of cells in a post-natal, preferably adult human. An antibody according to the present invention is typically capable of binding EGFR and ErbB-3 with a binding affinity (i.e. equilibrium dissociation constant KD) of at least 1×10e-6 M, as outlined in more detail below.

As used herein, antigen-binding refers to the typical binding capacity of an antibody to its antigen. An antibody comprising an antigen-binding site that binds to EGFR, binds to EGFR and, under otherwise identical conditions, at least 100-fold lower to the homologous receptors ErbB-2 and ErbB-4 of the same species. An antibody comprising an antigen-binding site that binds to ErbB-3, binds to ErbB-3 and, under otherwise identical conditions, not to the homologous receptors ErbB-2 and ErbR-4 of the same species. Considering that the ErbB-family is a family of cell surface receptors, the landing is typically assessed on cells that express the receptor(s). Binding of an antibody to an antigen can be assessed in various ways.

The term “interferes with binding” as used herein means that the antibody is directed to an epitope on ErbB-3 and the antibody competes with ligand for binding to ErbB-3. The antibody may diminish ligand binding, displace ligand when this is already bound to ErbB-3 or it may, for instance through steric hindrance, at least partially prevent that ligand can bind to ErbB-3. One way to measure binding of an antigen binding site is to incubate the antibody with the antigen (preferably cells expressing the antigen), removing unbound antibody (preferably by a wash step) and detecting bound antibody by means of a labeled antibody that binds to the bound antibody constant domain. The measurement is preferably compared with a positive and negative reference. In case of cells the negative reference is a cell that does not express the antigen.

The term “antibody” as used herein means a proteinaceous molecule preferably belonging to the immunoglobulin class of proteins, containing one or more variable domains that bind an epitope on an antigen, where such domains are derived from or share sequence homology with the variable domain of an antibody.

Antibodies of the invention preferably comprise two variable domains. Antibodies for therapeutic use are preferably as close to natural antibodies of the subject to be treated as possible (for instance human antibodies for human subjects) Antibody binding can be expressed in terms of specificity and affinity. The specificity determines which antigen or epitope thereof is specifically bound by the binding domain. The affinity is a measure for the strength of binding to a particular antigen or epitope. Specific binding, is defined as binding with affinities (KD) of at least 1×10e-6 M, more preferably 1×10e-7 M, more preferably higher than 1×10e-9 M. Typically, antibodies for therapeutic applications have affinities of up to 1×10e-10 M or higher. Antibodies such as bispecific antibodies of the present invention comprise the constant domains (Fc part) of a natural antibody. An antibody of the invention is typically a bispecific full length antibody, preferably of the human IgG subclass. Preferably, the antibodies of the present invention are of the human IgG1 subclass. Such antibodies of the invention have good ADCC properties, have favorable half life upon in vivo administration to humans and CH3 engineering technology exists that can provide for modified heavy chains that preferentially form hetero-dimers over homo-dimers upon co-expression in clonal cells. For instance, ADCC activity of an antibody can be improved when the antibody itself has a low ADCC activity, by slightly modifying the constant region of the antibody (Junttila, T. T., K. Parsons, et al. (2010). “Superior In vivo Efficacy of Afucosylated Trastuzumab in the Treatment of HER2-Amplified Breast Cancer.” Cancer Research 70(11): 4481-4489)

An antibody of the invention is preferably a “full length” antibody. The term “full length” according to the invention is defined as comprising an essentially complete antibody, which however does not necessarily have all functions of an intact antibody. For the avoidance of doubt, a full length antibody contains two heavy and two light chains. Each chain contains constant (C) and variable (V) regions, which can be broken down into domains designated CH1, CH2, CH3, VH, and CL, VL. An antibody binds to antigen via the variable domains contained in the Fab portion, and after binding can interact with molecules and cells of the immune system through the constant domains, mostly through the Fc portion. The terms ‘variable domain’, ‘VH/VL pair’, ‘VH/VL’ are used herein interchangeably. Full length antibodies according to the invention encompass antibodies wherein mutations may be present that provide desired characteristics. Such mutations should not be deletions of substantial portions of any of the regions. However, antibodies wherein one or several amino acid residues are deleted, without essentially altering the binding characteristics of the resulting antibody are embraced within the term “full length antibody”. For instance, an IgG antibody can have 1-20 amino acid residue insertions, deletions or a combination thereof in the constant region.

Full length IgG antibodies are preferred because of their favorable half life and the need to stay as close to fully autologous (human) molecules for reasons of immunogenicity. An antibody of the invention is preferably a bispecific IgG antibody, preferably a bispecific full length IgG1 antibody. IgG1 is favored based on its long circulatory half life in man. It is preferred that the bispecific IgG antibody according to the invention is a human IgG1.

The term ‘bispecific’ (bs) means that one part of the antibody (as defined above) binds to one epitope on an antigen whereas the second part binds to a different epitope on the antigen, or on a different antigen. The different epitope is typically present on a different antigen. According to the present invention, said first and second antigens are in fact two different proteins. A preferred bispecific antibody is an antibody that comprises parts of two different monoclonal antibodies and consequently binds to two different types of antigen. One arm of the bispecific antibody typically contains a variable domain of one antibody and the other arm contains a variable domain of another antibody. The heavy chain variable regions of the bispecific antibody of the invention are different from each other, whereas the light chain variable regions are preferably the same in the bispecific antibodies of the invention, i.e. the bispecific antibodies of the invention are preferably composed of two parental antibodies that have the same light chain (i.e. common light chain antibodies). A bispecific antibody wherein the different heavy chain variable regions are associated with the same, or a common, light chain is also referred to as a bispecific antibody with a common light chain. Further provided is therefore a bispecific antibody according to the invention, wherein both arms comprise a common light chain.

Preferred bispecific antibodies can be obtained by co-expression of two different heavy chains and a common light chain in a single cell. When wildtype CH3 domains are used, co-expression of two different heavy chains and a common light chain will result in three different species, AA, AB and BB. To increase the percentage of the desired bispecific product (AB) CH3 engineering can be employed or in other words, one can use heavy chains with compatible hetero-dimerization domains as defined hereunder.

The term ‘compatible hetero-dimerization domains’ as used herein refers to protein domains that are engineered such that engineered domain A′ will preferentially form hetero-dimers with engineered domain B′ and vice versa, whereas homo-dimerization between A′-A′ and B′-B′ is disfavoured.

The term ‘common light chain’ according to the invention refers to light chains which may be identical or have some amino acid sequence differences while the binding specificity of the full-length antibody is not affected. It is for instance possible within the scope of the definition of common light chains as used herein, to prepare or find light chains that are not identical but still functionally equivalent, e.g., by introducing and testing conservative amino acid changes, changes of amino acids in regions that do not or only partly contribute to binding specificity when paired with the heavy chain, and the like. The terms ‘common light chain’, ‘common VL’, ‘single light chain’, ‘single VL’, with or without the addition of the term ‘rearranged’ are all used herein interchangeably. It is an aspect of the present invention to use as common light chain a human light chain that can combine with different heavy chains to form antibodies with functional antigen binding domains (WO2004/009618, WO2009/157771, Merchant et al. 1998, Nissim et al. 1994). Preferably, the common light chain has a germline sequence. A preferred germline sequence is a light chain variable region that is frequently used in the human repertoire and has good thermodynamic stability, yield and solubility. A preferred germline light chain is based on O12, preferably it is the rearranged germline human kappa light chain IgVκ1-39*01/IGJκ1*01 or a fragment or a functional equivalent (i.e. same IgVκ1-39 gene segment but different IGJκ gene segment) thereof (nomenclature according to the IMGT database worldwide web at imgt.org). Further provided is therefore a bispecific antibody according to the invention, wherein said common light chain is a germline light chain, preferably a rearranged germline human kappa light chain comprising the IgVKI-39 gene segment, most preferably the rearranged germline human kappa light chain IgVK1-39*01/IGJK1*01. The terms rearranged germline human kappa light chain IgVκ1-39*01/IGJκ1*01, IGKV1-39/IGKJ1, huVκ1-39 light chain or in short huVκ1-39 are used interchangeably throughout the application. Obviously, those of skill in the art will recognize that “common” also refers to functional equivalents of the light chain of which the amino acid sequence is not identical. Many variants of said light chain exist wherein mutations (deletions, substitutions, additions) are present that do not materially influence the formation of functional binding regions. The light chain of the present invention can also be a light chain as specified herein above, having 1-5 amino acid insertions, deletions, substitutions or a combination thereof.

Also contemplated are antibodies wherein a VH is capable of specifically recognizing a first antigen and the VL, paired with the VH in a immunoglobulin variable domain, is capable of specifically recognizing a second antigen. The resulting VH/VL pair will bind either antigen 1 or antigen 2. Such so called “two-in-one antibodies”, described in for instance WO 2008/027236, WO 2010/108127 and Schaefer et al (Cancer Cell 20, 472-486. October 2011), are different from bispecific antibodies of the invention and are further referred to as “two-in-one antibodies”. Such “two-in-one” antibodies have identical arms and are not antibodies of the present invention.

EGFR is a member of a family of four receptor tyrosine kinases (RTKs), named Her- or cErbB-1, -2, -3 and -4. The EGFR has an extracellular domain (ECD) that is composed of four sub-domains, two of which are involved in ligand binding and one of which is involved in homo-dimerisation and hetero-dimerisation^{1,2} (for review, see Ref. 3). The reference numbers used in this section refer to the numbering of the references in the list headed “References cited in the specification”. EGFR integrates extracellular signals from a variety of ligands to yield diverse intracellular responses.^{4,5} The major signal transduction pathway activated by EGFR is composed of the Ras-mitogen-activated protein kinase (MAPK) mitogenic signalling cascade. Activation of this pathway is initiated by the recruitment of Grb2 to tyrosine phosphorylated EGFR.^{6,7} This leads to activation of Ras through the Grb2-bound Ras-guanine nucleotide exchange factor Son of Sevenless (SOS). In addition, the PI3-kinase-Akt signal transduction pathway is also activated by EGFR, although this activation is much stronger in case there is co-expression of Her3.^{6,8} The EGFR is implicated in several human epithelial malignancies, notably cancers of the breast, bladder, non-small cell lung cancer lung, colon, ovarian head and neck and brain.¹⁰ Activating mutations in the gene have been found, as well as over-expression of the receptor and of its ligands, giving rise to autocrine activation loops (for review, see Ref. 11). This RTK bas therefore been extensively used as target for cancer therapy. Both small-molecule inhibitors targeting the RTK and monoclonal antibodies (mAbs) directed to the extracellular ligand-binding domains have been developed and have shown hitherto several clinical successes, albeit mostly for a select group of patients.¹² Database accession numbers for the human EGFR protein and the gene encoding it are (GenBank NM_005228.3). The accession number is primarily given to provide a further method of identification of EGFR protein as a target, the actual sequence of the EGFR protein bound by an antibody may vary, for instance because of a mutation in the encoding gene such as those occurring in some cancers or the like. Where reference herein is made to EGFR, the reference refers to human EGFR unless otherwise stated. The antigen-binding site that binds EGFR, binds EGFR and a variety of variants thereof such as those expressed on some EGFR positive tumors.

The term ‘ErbB-3’ as used herein refers to the protein that in humans is encoded by the ERBB3 gene. Alternative names for the gene or protein are HER3: LCCS2: MDA-BF-1: c-ErbB-3; c-ErbB3; ErbB3-S; p180-ErbB3; p45-sErbB3; and p85-sErbB3. Where reference is made herein to ErbB-3, the reference refers to human ErbB-3. An antibody comprising an antigen-binding site that binds ErbB-3, binds human ErbB-3. The ErbB-3 antigen-binding site may, due to sequence and tertiary structure similarity between human and other mammalian orthologs, also bind such an ortholog but not necessarily so. Database accession numbers for the human ErbB-3 protein and the gene encoding it are (NP_001005915.1 NP_001978.2, NC_000012.11, NC_018928.2, NT_029419.12). The accession numbers are primarily given to provide a further method of identification of ErbB-3 as a target, the actual sequence of the ErbB-3 protein bound by an antibody may vary, for instance because of a mutation in the encoding gene such as those occurring in some cancers or the hike. The ErbB-3 antigen binding site binds ErbB-3 and a variety of variants thereof, such as those expressed by some ErbB-2 positive tumor cells. The antigen-binding site that binds ErbB-3 preferably binds domain III of ErbB-3. In a preferred embodiment the affinity (KD) of an antigen-binding site for an ErbB-3 positive cell is lower than or equal to 2.0 nM, more preferably lower than or equal to 1.5 nM, more preferably lower than or equal to 1.89 nM, more preferably lower than or equal to 0.99 nM. In a preferred embodiment, an antibody according to the invention preferably comprises an antigen-binding site that binds at least one amino acid of domain III of ErbB-3 selected from the group consisting of R426 and surface-exposed amino acid residues that are located within 11.2 Å from R426 in the native ErbB-3 protein. In one preferred embodiment, the affinity (KD) of an antigen-binding site for ErbB-3 on SK-BR-3 cells is lower than or equal to 2.0 nM, more preferably lower than or equal to 1.5 nM, more preferably lower than or equal to 1.39 nM, preferably lower than or equal to 0.99 nM. In one embodiment, said affinity (KD) is within the range of 1.39-0.59 nM. In one preferred embodiment, the affinity (KD) an antigen-binding site for ErbB-3 on BT-474 cells is lower than or equal to 2.0 nM, more preferably lower than or equal to 1.5 nM, more preferably lower than or equal to 1.0 nM, more preferably lower than 0.5 nM, more preferably lower than or equal to 0.31 nM, more preferably lower than or equal to 0.23 nM. In one embodiment, said affinity (KD) is within the range of 0.31-0.15 nM. The above-mentioned affinities are preferably as measured using steady state cell affinity measurements, wherein cells are incubated at 4° C. using radioactively labeled antibody, where after cell-bound radioactivity is measured, as described in the Examples.

An antigen-binding site that binds at least one amino acid of domain III of ErbB-3 preferably binds an amino acid selected from the group consisting of R426 and surface-exposed amino acid residues that are located within 11.2 Å from R426 in the native ErbB-3 protein. The amino acid residue numbering is that of Protein Data Bank (PDB) ID #4P59. As shown in the Examples, antibodies binding this region of domain III of ErbB-3 exhibit particularly good binding characteristics and they are capable of counteracting an activity of ErbB-3 on ErbB-3 positive cells. The term “surface-exposed amino acid residues that are located within 11.2 Å from R426 in the native ErbB-3 protein” refers to amino acid residues that are in the tertiary structure of the ErbB-3 protein spationally positioned within 11.2 Å from R426 and that are at least in part exposed to the outside of the protein, so that they can be reached by antibodies. Preferably, said amino acid residues that are located within 11.2 Å from R426 in the native ErbB-3 protein are selected from the group consisting of L423, Y424, N4285, G427, G452, R453, Y455. E480, R481, L482, D483 and K485 (see for instance FIG. 16 and Table 8). In one preferred embodiment, a bispecific antibody according to the invention is provided, wherein said antibody comprises an antigen-binding site that binds at least R426 of domain III of ErbB-3. Preferably, said antibody comprises an antigen-binding site that binds at least R426 of domain III of ErbB-3.

The invention further provides a bispecific antibody comprising an antigen-binding site that binds at least R426 of domain III of ErbB-3. Preferably, said antibody comprises an antigen-binding site that binds at least R426 of domain III of ErbB-3. Preferably said antibody further comprises a variable region as depicted in FIG. 11B. In a preferred embodiment the antibody further comprises a binding site for EGFR. The variable region preferably comprises a sequence as depicted in FIG. 11a.

A bispecific antibody of the invention preferably has improved ADCC activity. One technique for enhancing ADCC of an antibody is afucosylation. (See for instance Junttila. T. T., K. Parsons, et al. (2010). “Superior In vivo Efficacy of Afucosylated Trastuzumab in the Treatment of HER2-Amplified Breast Cancer.” Cancer Research 70(11): 4481-4489). Further provided is therefore a bispecific antibody according to the invention, which is afucosylated. Alternatively, or additionally, multiple other strategies can be used to achieve ADCC enhancement, for instance including glycoengineering (Kyowa Hakko/Biowa, GlycArt, (Roche) and Eureka Therapeutics) and mutagenesis (Xencor and Macrogenics), all of which seek to improve Fe binding to low-affinity activating FcγRIIIa, and/or to reduce binding to the low affinity inhibitory FcγRIIb. A bispecific antibody of the invention is preferably afucosylated in order to enhance ADCC activity. A bispecific antibody of the invention preferably comprises a reduced amount of fucosylation of the N-linked carbohydrate structure in the Fc region, when compared to the same antibody produced in a normal CHO cell.

The invention provides a bispecific antibody comprising a first antigen-binding site that binds EGFR and a second antigen-binding site that bind Erb B-3, wherein the antibody has a half maximal growth inhibitory concentration (IC50) of less than 200 pM for inhibiting EGFR and/or ErbB-3 ligand induced growth of BxPC3 cells (ATCC CRL-1687) or BxPC3-luc2 cells (Perkin Elmer 125058). Said antibody preferably has an IC50 for inhibiting EGFR and/or ErbB-3 ligand induced growth of BxPC3 cells (ATCC CRL 1687) or BxPC3-luc2 cells (Perkin Elmer 125058) of less than 100 pM, preferably Ines than 50 pM, more preferably less than 20 pM. An antibody of the invention preferably has an IC50 of more than 1 pM for inhibiting EGFR and/or ErbB-3 ligand induced growth of BxPC3 cells (ATCC CRL 1687) or BxPC3-luc2 cells (Perkin Elmer 125058).

The invention further provides a bispecific comprising a first antigen-binding site that binds EGFR and a second antigen-binding site that binds ErbB-3, wherein the antibody has a half maximal growth inhibitory concentration (IC50) for inhibiting EGFR and/or ErbB-3 ligand induced growth of BxPC3 cells (ATCC CRL-1687) or BxPC3-luc2 cells (Perkin Elmer 125058) that is lower than the IC50 of the antibody MEHD7945A for inhibiting growth of those cells under otherwise the same conditions. The anti-EGFR/anti-HER3 antibody MEHD7945A is described in WO2010/108127. Preferably the IC50 for inhibiting EGFR and/or ErbB-3 ligand induced growth of BxPC3 cells or BxPC3-luc2 cells of an antibody of the invention is lower than 90% of the IC50 of MEHD7945A for inhibiting growth of these cells under otherwise the same conditions, preferably lower than 80%, more preferably lower than 60%, more preferably lower than 50%, more preferably lower than 40%, more preferably lower than 30%, more preferably lower than 20%, more preferably lower than 10% of the IC50 of the antibody MEHD7945A. An antibody of the invention preferably has an IC50 for inhibiting EGFR and/or ErbB-3 ligand induced growth of BxPC3 cells (ATCC CRL 1687) or BxPCR3-luc2 cells (Perkin Elmer 125058) of more than 1% of the IC50 of the antibody MEHD7945A for inhibiting growth of those cells under otherwise the same conditions. An antibody of the invention preferably has the indicated IC50 for inhibiting EGFR and ErbB-3 ligand induced growth of BxPC3 cells (ATCC CRL 1687) or BxPC3-luc2 cells (Perkin Elmer 125058).

An antibody that is effective at relatively low concentrations of the antibody is preferred in the present invention. Such an antibody can be provided in lower amounts and/or with a lower frequency of administration making the utility of the antibody more economic. An antibody that is effective at relatively low concentrations of the antibody more effectively inhibits proliferation of tumor cells in vivo, particularly at lower concentrations. Such an antibody also has a better therapeutic window, and can be administered less frequently or with longer administration intervals.

EGFR and ErbB-3 each can bind a number of ligands and stimulate growth of BxPC3 cells or BxPC3-luc2 cells. In the presence of a ligand for one or both receptors the growth of BxPC3 or BxPC3-luc2 cells is stimulated. EGFR and/or ErbB-3 ligand-induced growth of BxPC3 cells can be measured by comparing the growth of the cells in the absence and presence of the ligand. The preferred EGFR ligand for measuring EGFR ligand-induced growth of BxPC3 or BxPC3-luc2 cells is EGF. The preferred ErbB-3 ligand for measuring ErbB-3 ligand-induced growth of BxPC3 or BxPC3-luc2 cells is NRG1. The ligand-induced growth is preferably measured using saturating amounts of ligand. In a preferred embodiment EGF is used in an amount of 100 ng/ml of culture medium. NRG1 is preferably used in 10 ng/ml of culture medium. It is preferred that the half maximal growth inhibitory concentration (IC50) is measured on EGFR and ErbB-3 ligand induced BxPC3 or BxPC3-luc2 cells. It is preferred that EGF is the EGFR-ligand in this assay and that NRG1 is the ErbB-3 ligand. A suitable test for the IC50 assay is described in the examples.

In the presence of excess ErbB-2, ErbB-2/ErbB-3 hetero-dimers may provide a growth signal to the expressing cell in the absence of detectable ligand for the ErbB-3 chain in the hetero-dimer. This ErbB-3 receptor function is herein referred as a ligand-independent receptor function of ErbB-3. The ErbB-2/Erb B-3 hetero-dimer also provide a growth signal to the expressing cell in the presence an ErbB-3 ligand. This ErbB-3 receptor function is herein referred to as a ligand-induced receptor function of ErbB-3.

EGF and NRG1 are preferably the EGF and NRG1 of R&D systems, cat. nr. 396-HB and 236-EG as described in the examples.

An antibody of the invention comprising an antigen-binding site that binds ErbB-3 preferably can reduce a ligand-induced receptor function of ErbB-3 on a ErbB-3 positive cell. The antibody is capable of reducing ErbB-3 signaling via dimerization with EGFR. The antibody is capable of reducing ErbB-3 signaling via ErbB-2. The ErbB-3 positive cell is preferably also positive for ErbB-2. The ErbB-3 positive cell is preferably also positive for EGFR. The ligand-induced receptor function of ErbB-3 is preferably ErbB-8 ligand-induced growth of an ErbB-2 and ErbB-3 positive cell. In a preferred embodiment the ErbB-2 and ErbB-3 positive cell comprises at least 50 000 ErbB-2 receptors on the cell surface. In a preferred embodiment at least 100.000 ErbB-2 receptors. In a preferred embodiment the ErbB-2 and ErbB-3 positive cell comprises no more than 1.000.000 ErbB-2 receptors on the cell surface. In a preferred embodiment the ErbB-2 and ErbB-3 positive cell comprises more than 1.000.000 ErbB-2 receptors on the cell surface. In a preferred embodiment said ErbB-2 and ErbB-3 positive cell is an BxPC3 cell (ATCC CRL-1687), a or BxPC3-luc2 cell (Perkin Elmer 126068); an MCF-7 cell (ATCC® HTB-22™), an SKBR3 cell (ATCC® HTB-30™) an NCI-87 cell (ATCC® CRL-5822™) or an A431 cell (ATCC® CRL-1555™). Preferably said ErbB-2 and ErbR-3 positive cell is also EGFR positive. Said ErbB-2 and ErbR-3 positive cell is preferably a BxPC3 or BxPC3-luc2 cell as indicated herein above.

As used herein the ligand-induced receptor function is reduced by at least 20%, preferably at least 30, 40, 50 60, or at least 70% in a particularly preferred embodiment the ligand-induced receptor function is reduced by 80, more preferably by 90%. The reduction is preferably determined by determining a ligand-induced receptor function in the presence of a bispecific antibody of the invention, and comparing it with the same function in the absence of the antibody, under otherwise identical conditions. The conditions comprise at least the presence of an ErbB-3 ligand. The amount of ligand present is preferably an amount that induces half of the maximum growth of an ErbB-2 and ErbB-3 positive cell line. The ErbB-2 and ErbB-3 positive cell line for this test is preferably the BxPC3 or BxPC3-luc2 cell as indicated herein above. The test and/or the ligand for determining ErbB-3 ligand-induced receptor function is preferably a test for ErbB-3 ligand induced growth reduction as specified in the examples,

An antibody of the invention comprising an antigen-binding site that binds ErbB-3, preferably interferes with binding of an ErbB-3 ligand to ErbB-3. Such antibodies are more effective in reducing ligand induced growth of BxPC3 or BxPC3-luc2 cells particularly in the context of an antibody that also comprises an antigen-binding site that binds EGFR.

The term “ErbB-3 ligand” as used herein refers to polypeptides which bind and activate ErbB-3. Examples of ErbB-3 ligands include, but are not limited to neuregulin 1 (NRG1) and neuregulin 2 (NRG2) (for review Olayioye M A et al.; EMBO J (2000) Vol 19: pp 8159-3167). The term includes biologically active fragments and/or variants of a naturally occurring polypeptide.

The term “EGFR ligand” as used herein refers to polypeptides which bind and activate EGFR. Examples of EGFR ligands include, but are not limited to EGF, TGF-α, HB-EGF, amphiregulin, betacellulin and epiregulin (for review Olayioye M A et al.; EMBO J (2000) Vol 19: pp 3159-3167). The term includes biologically active fragments and/or variants of a naturally occurring polypeptide

The first antigen-binding site of an antibody of the invention preferably binds domain For domain III of EGFR. Preferably said antibody binds domain III of EGFR. The antibody preferably inhibits EGF induced proliferation of BxPC3 or BxPC3-luc2 cells.

A bispecific antibody of the invention preferably comprises antibody-dependent cell-mediated cytotoxicity (ADCC). An antibody that has a low intrinsic ADCC activity can be provided with additional ADCC activity. The antibody can be engineered to enhance the ADCC activity (for review, see Cancer Sci. 2009 September: 100(9):1566-72. Engineered therapeutic antibodies with improved effector functions. Kubota T, Niwa R. Satoh M, Akinaga S. Shitara K. Hanai N). Several in vitro methods exist for determining the efficacy of antibodies or effector cells in eliciting ADCC. Among those are chromium-51 [Cr51] release assays, europium [Eu] release assays, and sulfur-35 [S35] release assays. Usually, a labeled target cell line expressing a certain surface-exposed antigen is incubated with antibody specific for that antigen. After washing, effector cells expressing Fe receptor CD16 are co-incubated with the antibody-labeled target cells. Target cell lysis is subsequently measured by release of intracellular label by a scintallation counter or spectrophotometry. A preferred test is detailed in the examples. A bispecific antibody of the invention is preferably afucosylated. A bispecific antibody of the invention preferably comprises a reduced amount of fucosylation of the N-linked carbohydrate structure in the Fc region, when compared to the same antibody produced in a normal CHO cell.

A bispecific antibody of the present invention is preferably used in humans. To this end an antibody of the invention is preferably a human or humanized antibody.

Tolerance of a human to a polypeptide is governed by many different aspects. Immunity, be it T-cell mediated, B-cell mediated or other is one of the variables that are encompassed in tolerance of the human for a polypeptide. The constant region of a bispecific antibody of the present invention is preferably a human constant region. The constant region may contain one or more, preferably not more than 10, preferably not more than 6 amino-acid differences with the constant region of a naturally occurring human antibody. It is preferred that the constant part is entirely derived from a naturally occurring human antibody. Various antibodies produced herein are derived from a human antibody variable domain library. As such these variable domains are human. The unique CDR regions may be derived from humans, be synthetic or derived from another organism. The variable region is considered a human variable region when it has an amino acid sequence that is identical to an amino acid sequence of the variable region of a naturally occurring human antibody, but for the CDR regions. The variable region of an EGFR binding VH, an ErbB-8 binding VH, or a light chain in an antibody of the invention may contain one or more, preferably not more than 10, preferably not more than 5 amino-acid differences with the variable region of a naturally occurring human antibody, not counting possible differences in the amino acid sequence of the CDR regions. Such mutations also occur in nature in the context of somatic hypermutation.

Antibodies may be derived from various animal species, at least with regard to the heavy chain variable region. It is common practice to humanize such e.g. murine heavy chain variable regions. There are various ways in which this can be achieved among which there are CDR-grafting into a human heavy chain variable region with a 3D-structure that matches the 3-D structure of the murine heavy chain variable region; deimmunization of the murine heavy chain variable region, preferably done by removing known or suspected T- or B-cell epitopes from the murine heavy chain variable region. The removal is typically by substituting one or more of the amino acids in the epitope for another (typically conservative) amino acid, such that the sequence of the epitope is modified such that it is no longer a T- or B-cell epitope.

Deimmunized murine heavy chain variable regions are loss immunogenic in humans than the original murine heavy chain variable region. Preferably a variable region or domain of the invention is further humanized, such as for instance veneered. By using veneering techniques, exterior residues which are readily encountered by the immune system are selectively replaced with human residues to provide a hybrid molecule that comprises either a weakly immunogenic or substantially non-immunogenic veneered surface. An animal as used in the invention is preferably a mammal, more preferably a primate, most preferably a human.

A bispecific antibody according to the invention preferably comprises a constant region of a human antibody. According to differences in their heavy chain constant domains, antibodies are grouped into five classes, or isotypes: IgG, IgA, IgM, IgD, and IgE. These classes or isotypes comprise at least one of said heavy chains that is named with a corresponding Greek letter. In a preferred embodiment the invention provides an antibody according to the invention wherein said constant region is selected from the group of IgG, IgA, IgM, IgD, and IgE constant regions, more preferably said constant region comprises an IgG constant region, more preferably an IgG1 constant region, preferably a mutated IgG1 constant region. Some variation in the constant region of IgG1 occurs in nature and/or is allowed without changing the immunological properties of the resulting antibody. Typically between about 1-10 amino acid insertions, deletions, substitutions or a combination thereof are allowed in the constant region.

The invention in one embodiment provides an antibody comprising a variable domain that binds EGFR, wherein said antibody comprises at least the CDR3 sequence of an EGFR specific heavy chain variable region selected from the group consisting of MF3998; MF4280; MF4002; MF4003; MF4010; MF4289; MF3370; MF3751, ME3752 as depicted in FIG. 11A, or wherein said antibody comprises a heavy chain CDR3 sequence that differs in at most three, preferably in at most two, preferably in no more than one amino acid from a CDR3 sequence of a VH selected from the group consisting of MF3998; MF4280; MF4002; MF4003; MF4010; MF4289; MF3370; MF3751; MF3752 as depicted in FIG. 11A. Said antibody preferably comprises at least the CDR3 sequence of MF3998; MF4280; MF4003; MF4010; MF4289; or MF3370 as depicted in FIG. 11A. Said antibody preferably comprises at least the CDR1, CDR2 and CDR3 sequences of an EGFR specific heavy chain variable region selected from the group consisting of MF3998; MF4280; MF4002; MF4003; MF4010; MF4289; MF3370; MF3751; MF3752 as depicted in FIG. 11A, or heavy chain CDR1, CDR2 and CDR3 sequences that differ in at most three, preferably in at most two, preferably in at most one amino acid from the CDR1, CDR2 and CDR3 sequences of EGFR specific heavy chain variable region selected from the group consisting of MF3998; MF4280; MF4002; MF4003; MF4010; MF4289; MF3370; MF3751; MF3752 as depicted in FIG. 11A. Said antibody preferably comprises at least the CDR1, CDR2 and CDR3 sequences of MF3998; MF4280; MF4003; MF4030; MF4280; or MF3370 as depicted in FIG. 11A.

The invention also provides an antibody comprising a variable domain that binds ErbB-3, wherein said antibody comprises at least the CDR3 sequence of an ErbB-3 specific heavy chain variable region selected from the group consisting of MF3178, MF3176, MF3163, MF3307, MF6055-MF6074, preferably MF3178, MF3176, MF3163, MF6058, MF6061 or MF6065 as depicted in FIG. 11B, or wherein said antibody comprises a heavy chain CDR3 sequence that differs in at most three, preferably in at most two, preferably in no more than one amino acid from a CDR3 sequence of a VH selected from the group consisting of MF3178, MF3176, MF3163, MF3307, MF6055-MF6074, preferably MF3178, MF3176, MF3163, MF6058, MF6061 or MF6065 as depicted in FIG. 11B. Said antibody preferably comprises at least the CDR3 sequence of MF3178, MF3176 or MF3163, most preferably at least the CDR3 sequence of MF3178. Said antibody preferably comprises at least the CDR1, CDR2 and CDR8 sequences of an ErbB3 specific heavy chain variable region selected from the group consisting of MF3178, MF3176, MF3163, MF3307, MF6055-MF6074, preferably MF3178, MF3176, MF3163, MF6058, MF6061 or MF6065 as depicted in FIG. 11B, or heavy chain CDR1, CDR2 and CDR3 sequences that differ in at most three, preferably in at most two, preferably in at most one amino acid from the CDR1, CDR2 and CDR3 sequences of MF3178, MF3176, MF3163, MF3307, MF6055-MF6074, preferably MF3178, MF3176, MF3163, MF6058, MF6061 or MF6065. Said antibody preferably comprises at least the CDR1, CDR2 and CDR3 sequences of MF3178, MF3176 or MF3163, most preferably at least the CDR1, CDR2 and CDR3 sequence of MF3178.

The invention in one embodiment provides a bispecific antibody comprising a first antigen-binding site that binds EGFR and a second antigen-binding site that binds ErbB-3, wherein said first antigen-binding site comprises at least the CDR3 sequence of an EGFR specific heavy chain variable region selected from the group consisting of MF3998; MF4280; MF4002; MF4003; MF4010; MF4289; MF3370; MF3751; MF3752 as depicted in FIG. 11A, or a heavy chain CDR3 sequence that differs in at most three, preferably in at most two, preferably in no more than one amino acid from a CDR3 sequence of a VH selected from the group consisting of MF3998; MF4280; MF4002; MF4003; MF4010; MF4289; MF3370; MF3751; MF3752 as depicted in FIG. 11A, and wherein said second antigen-binding site comprises at least the CDR3 sequence of an ErbB 3 specific heavy chain variable region selected from the group consisting of MF3178, MF3176, MF3163, MF3307, MF6055-MF6074, preferably MF3178, MF3176, MF3163, MF6058, MF6061 or MF6065 as depicted in FIG. 16B or FIG. 16E, or a heavy chain CDR3 sequence that differs in at most three, preferably in at most two, preferably in no more than one amino acid from a CDR3 sequence of a VH selected from the group consisting of MF3178, MF3176, MF3163, MF3307, MF6055-MF6074, preferably MF3178, MF3176, MF3163, MF6058, MF6061 or MF6065 as depicted in FIG. 11B. Said first antigen-binding site preferably comprises at least the CDR3 sequence of MF3998; MF4280, MF4003; MF4010; MF4289, or MF3370 as depicted in FIG. 11A and said second antigen-binding site preferably comprises at least the CDR3 sequence of MF3178, MF3176 or MF3163, most preferably at least the CDR3 sequence of MF3178 of FIG. 11B. Said first antigen-binding site preferably comprises at least the CDR1, CDR2 and CDR3 sequences of an EGFR specific heavy chain variable region selected from the group consisting of MF3998; MF4280; MF4003; MF4010; MP4289; or MF3370 as depicted in FIG. 11A, or heavy chain CDR1, CDR2 and CDR3 sequences that differ in at most three, preferably in at most two, preferably in at most one amino acid from the CDR1, CDR2 and CDR3 sequences of MF3998; MF4280; MF4003, MF4010; MF4289; or MF3370 as depicted in FIG. 11A, and said second antigen-binding site preferably comprises at least the CDR1, CDR2 and CDR3 sequences of an ErbB 3 specific heavy chain variable region selected from the group consisting of MF3178, MF3176, MF3163, MF3307, MF6055-MF6074, preferably MF3178, MF3176, MF3163, MF6058, MF6061 or MF6065 as depicted in FIG. 11B, or heavy chain CDR1, CDR2 and CDR3 sequences that differ in at most three, preferably in at most two, preferably in at most one amino acid from the CDR1, CDR2 and CDR3 sequences of MF3178, MP3176, MF3163, MF3307, MF6055-MF6074, preferably MF3178, MF3176, MF3163, MF6058, MF6061 or MF6065. Said first antigen-binding site preferably comprises at least the CDR1, CDR2 and CDR3 sequences of MF3998; MF4280; ME4003; MF4010; MF4289; or MF3370 as depicted in FIG. 11A, and said second antigen-binding site preferably comprises at least the CDR1, CDR2 and CDR3 sequences of MF3178, MF3176 or MF3163, most preferably at least the CDR1, CDR2 and CDR3 sequence of MF3178.

A preferred embodiment provides a bispecific antibody comprising a first antigen-binding site that binds EGFR and a second antigen-binding site that binds ErbB-3, wherein said first antigen-binding site comprises at least the CDR3 sequence of MF3998 as depicted in FIG. 11A, or a CDR3 sequence that differs in at most three, preferably in at most two, preferably in no more than one amino acid from the CDR3 sequence of MF3998 as depicted in FIG. 11A, and wherein said second antigen-binding site comprises at least the CDR3 sequence of MF3178, or a CDR3 sequence that differs in at most three, preferably in at most two, preferably in no more than one amino acid from the CDR3 sequence of MF3178.

The invention in one embodiment provides a bispecific antibody comprising a first antigen-binding site that binds EGFR and a second antigen-binding site that binds ErbB-3, wherein said first antigen-binding site comprises at least the CDR1, CDR2 and CDR3 sequences of MF3998, or CDR1, CDR2 and CDR3 sequences that differ in at most three, preferably in at most two, preferably in at most one amino acid from the CDR1, CDR2 and CDR3 sequences of MF3958, and wherein said second antigen-binding site comprises at least the CDR1, CDR2 and CDR3 sequence of MF3178, or CDR1, CDR2 and CDR3 sequences that differ in at most three, preferably in at most two, preferably in at most one amino acid from the CDR1, CDR2 and CDR3 sequences of MF3178.

The invention in one embodiment provides a bispecific antibody comprising a first antigen-binding site that binds EGFR and a second antigen-binding site that binds ErbB-3, wherein said first antigen-binding site comprises at least the CDR3 sequence of MF3998 and wherein said second antigen-binding site comprises at least the CDR3 sequence of MF3178.

The invention in one embodiment provides a bispecific antibody comprising a first antigen-binding site that binds ErbB-2 and a second antigen-binding site that binds ErbB-3, wherein said first antigen-binding site comprises at least the CDR1, CDR2 and CDR8 sequences of MF3998 and wherein said second antigen-binding site comprises at least the CDR1, CDR2 and CDR3 sequence of MF3178.

The invention in one embodiment provides a bispecific antibody comprising a first antigen-binding site that binds EGFR and a second antigen-binding site that binds ErbB-3, wherein said first antigen-binding site comprises at least the CDR3 sequence of MF4280 and wherein said second antigen-binding site comprises at least the CDR3 sequence of MF3178.

The invention in one embodiment provides a bispecific antibody comprising a first antigen-binding site that binds ErbB-2 and a second antigen-binding site that binds ErbB-3, wherein said first antigen-binding site comprises at least the CDR1, CDR2 and CDR3 sequences of MF4280 and wherein said second antigen-binding site comprises at least the CDR1, CDR2 and CDR3 sequence of MF3178.

CDR sequences are for instance varied for optimization purposes, preferably in order to improve binding efficacy or the stability of the antibody. Optimization is for instance performed by mutagenesis procedures where after the stability and/or binding affinity of the resulting antibodies are preferably tested and an improved EGFR or ErbB 3-specific CDR sequence is preferably selected. A skilled person is well capable of generating antibody variants comprising at least one altered CDR sequence according to the invention. For instance, conservative amino acid substitution is applied. Examples of conservative amino acid substitution include the substitution of one hydrophobic residue such as isoleucine, valine, leucine or methionine for another hydrophobic residue, and the substitution of one polar residue for another polar residue, such as the substitution of arginine for lysine, glutamic acid for aspartic acid, or glutamine for asparagine.

The invention in one embodiment provides an antibody comprising a variable domain that binds EGFR, wherein the VH chain of said variable domain comprises the amino acid sequence of VH chain MF3998; MF4280; MF4002; MF4003; MF4010; MF4289; MF3370; MF3751; MF3752 as depicted in FIG. 11A; or comprises the amino acid sequence of VH chain MF3998; MF4280; MF4002; MF4003; MF4010; ME4289; MF3370; MF3751; MF3752 depicted in FIG. 11A having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain sequence of FIG. 11A. Said antibody preferably comprises a variable domain that binds EGFR, wherein the VH chain of said variable domain comprises the amino acid sequence of VH chain MF3998; MF4280; MF4003; MP4010; MF4289; or MF3370 as depicted in FIG. 11A; or comprises the amino acid sequence of VH chain MF3998; MF4280; MF4003; MF4010; MF4289; or MF3370 as depicted in FIG. 11A having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain sequence of FIG. 11A. An antibody comprising a variable domain that binds EGFR, preferably further comprises a variable domain that binds ErbB-3. The antibody comprising a variable domain that binds EGFR is preferably a bispecific antibody that preferably further comprises a variable domain that binds ErbB-3. The VH chain of the variable domain that binds Erb-B3 preferably comprises the amino acid sequence of VH chain MF3178; MF3170; MF3163; MF3307, MF6055-MF6074, preferably MF3178, MF3176, MF3163, MF6058, MF6061 or MF6065 as depicted in FIG. 11B; or comprises the amino acid sequence of VH MF3178; MF3176; MF3163; MF3307, MP6055-MF6074, preferably MF3178, MF3176, MF3163, MF6058, MF6061 or MF6065 depicted in FIG. 11B having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain sequence of FIG. 11B. The VH chain of the variable domain that binds Erb-B3 preferably comprises the amino acid sequence of MF3178, MF3176 or MF3163; or comprises the amino acid sequence of MF3178, MF3176 or MF3163 depicted in FIG. 11B having at most 15, preferably 1, 2, 3, 4, 5, 6, 7, 8, 9 or 10 and preferably having 1, 2, 3, 4 or 5 amino acid insertions, deletions, substitutions or a combination thereof with respect to the respective VH chain sequence of FIG. 11A. In a preferred embodiment the VH chain of the variable domain that binds ErbB-3 comprises the amino acid sequence of MF3178; or comprises the amino acid sequence of MF3178 depicted in FIG. 11B having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain sequence.

Preferably, the mentioned amino acid insertions, deletions and substitutions in a VH or VL as specified herein are not present in the CDR3 region. The mentioned amino acid insertions, deletions and substitutions are also preferably not present in the CDR1 and CDR2 regions. The mentioned amino acid insertions, deletions and substitutions are also preferably not present in the FR4 region.

The invention further provides an antibody comprising a variable domain that binds ErbB-3, wherein the VH chain of said variable region comprises the amino acid sequence of VH chain MF3178; MF3176; MF3163 or MF3307 as depicted in FIG. 11B, or comprises the amino acid sequence of VH MF3178; MF3176; MF3163; MF3307; MF6055-MF6074, preferably MF3178, MF3176, MF3163, MP6058, MF6061 or MF6065 depicted in FIG. 11B having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain sequence of FIG. 11B. The VH chain of the variable domain that binds ErbB3 preferably comprises the amino acid sequence of VH chain MF3178, MF3176 or MF3163; or comprises the amino acid sequence of VH chain MF3178, MF3176 or MF3168 depicted in FIG. 11B having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain sequence of FIG. 11B. In a preferred embodiment the VH chain of the variable domain that binds ErbB-3 comprises the amino acid sequence of VH chain MF3178 depicted in FIG. 11B; or comprises the amino and sequence of VH chain MF3178 depicted in FIG. 11B having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain sequence. The antibody preferably further comprises a variable domain that binds EGFR. The VH chain of the variable domain that binds EGFR preferably comprises an amino acid sequence of VH chain of FIG. 11A.

Further provided is an antibody according to the invention, wherein said antibody comprises an EGFR specific heavy chain variable region sequence selected from the group consisting of the heavy chain variable region sequences of MF3998; MF4280; MF4002; MF4003; MF4010; MF4289; MF3370; MF3751; MF3752 as depicted in FIG. 11A, or wherein said antibody comprises a heavy chain variable region sequence that differs in at most 15 amino acids from the heavy chain variable region sequences of MF3998; MF4280; MF4002; MF4003; MF4010; MF4289; MF3370; MF3751; MF3752 as depicted in FIG. 11A.

Further provided is an antibody according to the invention, wherein said antibody comprises an ErbB 3 specific heavy chain variable region sequence selected from the group consisting of the heavy chain variable region sequences of MF3178, MF3176, MF3163, MF3307, MF6055-MF6074, preferably MF3178, MF3176, MF3163, MF6058, MP6061 or MF6065 as depicted in FIG. 11B, or wherein said antibody comprises a heavy chain variable region sequence that differs in at most 15 amino acids from the heavy chain variable region sequences of MF3178, MF3176, MF3163, MF3307, MF6055-MF6074, preferably MF3178, MF3176, MF3163, MF6058, MF6061 or MF6065.

The invention further provides an antibody comprising two variable domains that each bind EGFR wherein a VH of the variable domains comprises the amino acid sequence of the VH chain MF3998; MF4280; MF4002; MF4003; MF4010; MP4289; MF3370; MF3751; MF3752 as depicted in FIG. 11A; or comprises the amino acid sequence of the VH chain MF3998; MF4280; MF4002; MF4003; MF4010; MF4289; MP3370; MF3751, MF3752 as depicted in FIG. 11A, wherein said VH-chain has at most 15, preferably 1, 2, 3, 4, 5, 6, 7, 8, 9 or 10 and preferably has 1, 2, 3, 4 or 5 amino acid insertions, deletions, substitutions or a combination thereof with respect to the VH chain sequence of FIG. 11A. Said VH preferably comprises the amino acid sequence of the VH chain MF3998; MF4280; MF4003; MF4010; MF4289; or MF3370 as depicted in FIG. 11A; or the amino acid sequence of the VH chain MF3998; MF4280; MF4008; MF4010; MF4289; or MF3370 as depicted in FIG. 11A, having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain sequence of FIG. 11A. The variable domains of the antibody preferably comprise identical VH chains, preferably having a sequence as depicted in FIG. 11A. An antibody with variable domains with identical VH chains is not a bispecific antibody. VH chains are identical for the present invention if they comprise the same VH chain sequence as depicted in FIG. 11, or the same VH chain sequence but for 1, 2, 3, 4 or 5 amino acid insertions, deletions, substitutions or a combination thereof with respect to the VH chain sequence of FIG. 11A.

The invention further provides an antibody comprising two variable domains that each bind ErbB3 wherein a VH of the variable domains comprises the amino acid sequence of VH chain MF3178; MF3176; MF3163 or MF3307 as depicted in FIG. 11B; or comprises the amino acid sequence of VH chain MF3178; MF3176; MF3163 or MF3307 depicted in FIG. 11B having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain sequence of FIG. 11B. Said VH preferably comprises the amino acid sequence of VH chain MF3178, MF3176 or MF3163; or comprises the amino acid sequence of VH chain MF3178, MF3176 or MF3163 depicted in FIG. 11B having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain sequence of FIG. 11B. Said VH preferably comprises the amino acid sequence of VH chain MF3178; or comprises the amino acid sequence of VH chain MF3178 depicted in FIG. 11B having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain sequence of FIG. 11B. The variable domains of the antibody preferably comprise identical VH chains, preferably having a sequence as depicted in FIG. 11B. An antibody with variable domains with identical VH chains is not a bispecific antibody. The VH chains are identical if they comprise the same VH chain sequence as depicted in FIG. 11B., or the same VH chain sequence but for 1, 2, 3, 4 or 6 amino acid insertions, deletions, substitutions or a combination thereof with respect to the VH chain sequence of FIG. 11B.

The invention preferably provides an antibody comprising a variable domain that binds EGFR and a variable domain that binds ErbB-3, wherein the VH chain of the variable domain that binds EGFR comprises
the amino acid sequence of VH chain MF3998 as depicted in FIG. 11; or
the amino acid sequence of VH chain MF3998 as depicted in FIG. 11 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and
   wherein the VH chain of the variable domain that binds ErbB-3 comprises
the amino acid sequence of VH chain MF3178 as depicted in FIG. 11; or
the amino acid sequence of VH chain MF3178 as depicted in FIG. 11 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention preferably provides an antibody comprising a variable domain that binds EGFR and a variable domain that binds ErbB-3, wherein the VH chain of the variable domain that binds EGFR comprises
the amino acid sequence of VH chain MF4280 as depicted in FIG. 11; or
the amino acid sequence of VH chain MF4280 as depicted in FIG. 11 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and
   wherein the VH chain of the variable domain that binds ErbB-3 comprises
the amino acid sequence of VH chain MF3178 as depicted in FIG. 11; or
the amino acid sequence of VH chain MF3178 as depicted in FIG. 11 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention preferably provides an antibody comprising a variable domain that binds EGFR and a variable domain that binds ErbB-3, wherein the VH chain of the variables domain that binds EGFR comprises
the amino acid sequence of VH chain MF4003 as depicted in FIG. 11; or
the amino acid sequence of VH chain MF4003 as depicted in FIG. 11 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and
   wherein the VH chain of the variable domain that binds ErbB-3 comprises
the amino acid sequence of VH chain MF3178 as depicted in FIG. 11; or
the amino acid sequence of VH chain MF3178 as depicted in FIG. 11 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention preferably provides an antibody comprising a variable domain that binds EGFR and a variable domain that binds ErbB-3, wherein the VH chain of the variable domain that binds EGFR comprises
the amino acid sequence of VH chain MF4010 as depicted in FIG. 11; or
the amino acid sequence of VH chain MF4010 as depicted in FIG. 11 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and
   wherein the VH chain of the variable domain that binds ErbB-3 comprises
the amino acid sequence of VH chain MF3178 as depicted in FIG. 11; or
the amino acid sequence of VH chain MF3178 as depicted in FIG. 11 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention preferably provides an antibody comprising a variable domain that binds EGFR and a variable domain that binds ErbB-3, wherein the VR chain of the variable domain that binds EGFR comprises
the amino acid sequence of VH chain MF4289 as depicted in FIG. 11; or
the amino acid sequence of VH chain MF4289 as depicted in FIG. 11 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and
   wherein the VH chain of the variable domain that binds ErbB-3 comprises
the amino acid sequence of VH chain MF3178 as depicted in FIG. 11; or
the amino acid sequence of VH chain MF3178 as depicted in FIG. 11 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH.

The invention preferably provides an antibody comprising a variable domain that binds EGFR and a variable domain that binds ErbB-3, wherein the VH chain of the variable domain that binds EGFR comprises
the amino acid sequence of VH chain MF3370 as depicted in FIG. 11; or
the amino acid sequence of VH chain MF3370 as depicted in FIG. 11 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH; and
   wherein the VH chain of the variable domain that binds ErbB-3 comprises
the amino acid sequence of VH chain MF3163 as depicted in FIG. 11; or the amino acid sequence of VH chain MF3163 as depicted in FIG. 11 having at most 15, preferably 1, 2, 3, 4, 5, 6, 7, 8, 9 or 10 and preferably having 1, 2, 3, 4 or 5 amino acid insertions, deletions, substitutions or a combination thereof with respect said VH. This antibody binds to human EGFR and murine EGFR and can be used to study target effects in mice.

When compared to the sequence in FIG. 11, the behavior of a VH chain typically starts to become noticeably different when it has more than 15 amino acid changes with respect to the amino acid sequence of a VH chain as depicted in FIG. 11. A VH chain having at most 15, preferably 1, 2, 3, 4, 5, 6, 7, 8, 9 or 10 amino acid insertions, deletions, substitutions or a combination thereof with respect to the VH chain depicted in FIG. 11, preferably has 1, 2, 3, 4 or 5 amino acid insertions, deletions, substitutions or a combination thereof with respect to the VH chain depicted in FIG. 11, preferably 1, 2, 3 or 4 insertions, deletions, substitutions or a combination thereof, preferably 1, 2 or 3 insertions, deletions, substitutions or a combination thereof, more preferably 1 or 2 insertions, deletions, substitutions or a combination thereof, and preferably 1 insertion, deletion, substitution or a combination thereof with respect to the VH chain depicted in FIG. 11. The one or more amino acid insertions, deletions, substitutions or a combination thereof are preferably not in the CDR1, CDR2 and CDR3 region of the VH chain. They are also preferably not present in the Fr4 region. An amino acid substitution is preferably a conservative amino acid substitution.

In a preferred embodiment the invention provides an antibody that has a heavy chain comprising an amino acid sequence as depicted in FIG. 11D, or an amino acid sequence of FIG. 11D having at most 15, preferably 1, 2, 3, 4, 5, 6, 7, 8, 9 or 10 and preferably having 1, 2, 3, 4 or 5 amino acid insertions, deletions, substitutions or a combination thereof with respect to the sequence of FIG. 11D. In a preferred embodiment the antibody has two heavy chains each comprising an amino acid sequence as depicted in FIG. 11D, or an amino acid sequence of FIG. 11D having at most 15, preferably 1, 2, 3, 4, 5, 6, 7, 8, 9 or 10 and preferably having 1, 2, 3, 4 or 5 amino acid insertions, deletions, substitutions or a combination thereof with respect to the sequence of FIG. 11D. The at most 15, preferably 1, 2, 3, 4, 5, 6, 7, 8, 9 or 10 and preferably 1, 2, 3 4 or 5 amino acid substitutions are preferably conservative amino acid substitutions. The insertions, deletions, substitutions or a combination thereof are preferably not in the CDR3 region of the VH chain, preferably not in the CDR1, CDR2 and CDR3 region of the VH chain and preferably not in the FR4 region.

Rational methods have evolved toward minimizing the content of non-human residues in the human context. Various methods are available to successfully graft the antigen-binding property of an antibody onto another antibody. The binding properties of antibodies rest predominantly in the exact sequence of the CDR3 region, often supported by the sequence of the CDR1 and CDR2 regions in the variable domain combined with the appropriate structure of the variable domain as a whole. Various methods are presently available to graft CDR regions onto a suitable variable domain of another antibody. Some of these methods are reviewed in J. C. Almagrol and J. Fransson (2008) Frontiers in Bioscience 13, 1619-1633, which is included by reference herein. The invention therefore further provides a human or humanized bispecific antibody comprising a first antigen-binding site that binds EGFR and a second antigen-binding site that binds ErbB-3, wherein the variable domain comprising the EGFR binding site comprises a VH CDR3 sequence as depicted in FIG. 11A, and wherein the variable domain comprising the ErbB-3 binding site comprises a VH CDR3 region as depicted in FIG. 11B. The VH variable region comprising the EGFR binding site preferably comprises the sequence of the CDR1 region. CDR2 region and the CDR3 region of a VH chain in FIG. 11A. The VH variable region comprising the ErbB-3 binding site preferably comprises the sequence of the CDR1 region, CDR2 region and the CDR3 region of a VH chain in FIG. 11B. CDR grafting may also be used to produce a VH chain with the CDR regions of a VH of FIG. 11, but having a different framework. The different framework may be of another human VH, or of a different mammal.

The mentioned at most 15, preferably 1, 2, 3, 4, 5, 6, 7, 8, 9 or 10 and preferably 1, 2, 3, 4 or 5 amino acid substitutions are preferably conservative amino acid substitutions, the insertions, deletions, substitutions or a combination thereof are preferably not in the CDR3 region of the VH chain, preferably not in the CDR1, CDR2 or CDR3 region of the VH chain and preferably not in the FR4 region.

The light chain of a variable domain comprising a variable heavy chain sequence as depicted in FIG. 11, is preferably a germline light chain of or based on O12, preferably the rearranged germline human kappa light chain IgVκ1-39*01/IGJκ1*01 or a fragment or a functional derivative thereof (nomenclature according to the IMGT database worldwide web at imgt.org). The terms rearranged germline human kappa light chain IgVκ1-39*01/IGJκ1*01, IGKV1-39/GKJ1, huVκ1-39 light chain or in short huVκ1-39 are used. The bight chain can have 1, 2, 3, 4 or 5 amino acid insertions, deletions, substitutions or combination thereof. The mentioned 1, 2, 3, 4 or 5 amino acid substitutions are preferably conservative amino acid substitutions, the insertions, deletions, substitutions or combination thereof are preferably not in the CDR3 region of the VL chain, preferably not in the CDR1, CDR2 or CDR3 region or FR4 region of the VL chain.

Various methods are available to produce bispecific antibodies. One method involves the expression of two different heavy chains and two different light chains in a cell and collecting antibody that is produced by the cell. Antibody produced in this way will typically contain a collection of antibodies with different combinations of heavy and light chains, some of which are the desired bispecific antibody. The bispecific antibody can subsequently be purified from the collection. The ratio of bispecific to other antibodies that are produced by the cell can be increased in various ways. In a preferred embodiment of the invention, the ratio is increased by expressing not two different light chains but two essentially identical light chains in the cell. This concept is in the art also referred to as the “common light chain” method. When the essentially identical light chains work together with the two different heavy chains allowing the formation of variable domains with different antigen-binding sites and concomitant different binding properties, the ratio of bispecific antibody to other antibody that is produced by the cell is significantly improved over the expression of two different light chains. The ratio of bispecific antibody that is produced by the cell can be further improved by stimulating the pairing of two different heavy chains with each other over the pairing of two identical heavy chains. The art describes various ways in which such hetero-dimerization of heavy chains can be achieved. One way is to generate ‘knob into bole’ bispecific antibodies. See US Patent Application 20030078385 (Arathoon et al. Genentech). Another method is by using charge engineering as described in Gunasekaran (JBC 2010, vol 285, pp 19637-19646). Another and preferred method is described in US provisional application 61/635,935, which has been followed up by U.S. application Ser. No. 13/866,747 and PCT application No. PCT/NL2013/050294 (WO 2013/157954 A1), which are incorporated herein by reference. Methods and means are disclosed for producing bispecific antibodies (from a single cell), whereby means are provided that favor the formation of bispecific antibodies over the formation of monospecific antibodies. These methods can also be favorably employed in the present invention. Thus the invention provides a method for producing a bispecific antibody according to the invention (from a single cell), wherein said bispecific antibody comprises two CH3 domains that are capable of forming an interface, said method comprising providing in said cell a) a first nucleic acid molecule encoding a 1st CH3 domain comprising heavy chain, b) a second nucleic acid molecule encoding a 2nd CH3 domain comprising heavy chain, wherein said nucleic acid molecules are provided with means for preferential pairing of said 1st and 2nd CH3 domain comprising heavy chains, said method further comprising the step of culturing said host cell and allowing for expression of said two nucleic acid molecules and harvesting said bispecific antibody from the culture. Said first and second nucleic acid molecules may be part of the same nucleic acid molecule, vector or gene delivery vehicle and may be integrated at the same site of the boat cell's genome. Alternatively, said first and second nucleic acid molecules are separately provided to said cell.

A preferred embodiment provides a method for producing a bispecific antibody according to the invention from a single cell, wherein said bispecific antibody comprises two CH3 domains that are capable of forming an interface, said method comprising providing:
a cell having a) a first nucleic acid molecule encoding a heavy chain comprising an antigen binding site that binds EGFR and that contains a 1st CH3 domain, and b) a second nucleic acid molecule encoding a heavy chain comprising an antigen-binding site that binds ErbB-3 and that contains a 2nd CH3 domain, wherein said nucleic acid molecules are provided with means for preferential pairing of said 1st and 2nd CH3 domains.
said method further comprising the step of culturing said cell and allowing for expression of the proteins encoded by said two nucleic acid molecules and harvesting said bispecific IgG antibody from the culture. In a particularly preferred embodiment, said cell also has a third nucleic acid molecule encoding a common light chain. Said first, second and third nucleic acid molecule may be part of the same nucleic acid molecule, vector or gene delivery vehicle and may be integrated at the same site of the host cell's genome. Alternatively, said first, second and third nucleic acid molecules are separately provided to said cell. A preferred common light chain is based on O12, preferably it is the rearranged germline human kappa light chain IgVκ1 39*01/1GJκ1*01, as described above. Means for preferential pairing of said 1^{st} and said 2^{nd} CH3 domain are preferably the corresponding mutations in the CH3 domain of the heavy chain coding regions. The preferred mutations to produce essentially only bispecific antibodies are the amino acid substitutions L351K and T366K (numbering according to Kabat) in the first CH3 domain and the amino acid substitutions L351D and L368E in the second CH3 domain, or vice versa. Further provided is therefore a method according to the invention for producing a bispecific antibody, wherein said first CH3 domain comprises the amino acid substitutions L351K and T366K (numbering according to Kabat) and wherein said second CH3 domain comprises the amino acid substitutions L351D and L368E, said method further comprising the step of culturing said cell and allowing for expression of proteins encoded by said nucleic acid molecules and harvesting said bispecific antibody from the culture. Also provided is a method according to the invention for producing a bispecific antibody, wherein said first CH3 domain comprises the amino acid substitutions L351D and L368E (numbering according to Kabat) and wherein said second CH3 domain comprises the amino acid substitutions L351K and T366K, said method further comprising the step of culturing said cell and allowing for expression of said nucleic acid molecules and harvesting said bispecific antibody from the culture. Antibodies that can be produced by these methods are also part of the present invention. The CH3 hetero-dimerization domains are preferably IgG1 hetero-dimerization domains. The heavy chain constant regions comprising the CH3 hetero-dimerization domains are preferably IgG1 constant regions.

In one embodiment the invention provides a nucleic acid molecule encoding an antibody heavy chain variable region according to the invention. The nucleic acid molecule (typically an in vitro, isolated or recombinant nucleic acid molecule) preferably encodes a heavy chain variable region as depicted in FIG. 11A or FIG. 11B, or a heavy chain variable region as depicted in FIG. 11A or FIG. 11B having 1, 2, 3, 4 or 5 amino acid insertions, deletions, substitutions or combination thereof. In a preferred embodiment the nucleic acid molecule comprises a sequence as depicted in FIG. 11. In another preferred embodiment the nucleic acid molecule encodes the same amino acid sequence as the nucleic acid depicted in FIG. 11 but has a different sequence because it encodes one or more different codons. The invention further provides a nucleic acid sequence encoding a heavy chain of FIG. 11D.

A nucleic acid molecule as used in the invention is typically but not exclusively a ribonucleic acid (RNA) or a deoxyribonucleic acid (DNA). Alternative nucleic acids are available for a person skilled in the art. A nucleic acid according to the invention is for instance comprised in a cell. When said nucleic acid is expressed in said cell, said cell can produce an antibody according to the invention. Therefore, the invention in one embodiment provides a cell comprising an antibody according to the invention and/or a nucleic acid according to the invention. Said cell is preferably an animal cell, more preferably a mammal cell, more preferably a primate cell, most preferably a human cell. For the purposes of the invention a suitable cell is any cell capable of comprising and preferably of producing an antibody according to the invention and/or a nucleic acid according to the invention.

The invention further provides a cell comprising an antibody according to the invention. Preferably said cell (typically an in vitro, isolated or recombinant cell) produces said antibody. In a preferred embodiment said cell is a hybridoma cell, a Chinese hamster ovary (CHO) cell, an NS0 cell or a PER-C6™ cell. In a particularly preferred embodiment said cell is a CHO cell. Further provided is a cell culture comprising a cell according to the invention. Various institutions and companies have developed cell lines for the large scale production of antibodies, for instance for clinical use. Non-limiting examples of such cell lines are CHO cells, NS0 cells or PER.C6™ cells. These cells are also used for other purposes such as the production of proteins. Cell lines developed for industrial scale production of proteins and antibodies are herein further referred to as industrial cell lines. Thus in a preferred embodiment the invention provides the use of a cell line developed for the large scale production of antibody for the production of an antibody of the invention. The invention further provides a cell for producing an antibody comprising a nucleic acid molecule that codes for a VH, a VL, and/or a heavy chain as depicted in FIG. 11. Preferably said nucleic acid molecule comprises a sequence as depicted in FIG. 11a or 11b.

The invention further provides a method for producing an antibody comprising culturing a cell of the invention and harvesting said antibody from said culture. Preferably said cell is cultured in a serum free medium. Preferably said cell is adapted for suspension growth. Further provided is an antibody obtainable by a method for producing an antibody according to the invention. The antibody is preferably purified from the medium of the culture. Preferably said antibody is affinity purified.

A cell of the invention is for instance a hybridoma cell line, a CHO cell, a 293F cell, an NS0 cell or another cell type known for its suitability for antibody production for clinical purposes. In a particularly preferred embodiment said cell is a human cell. Preferably a cell that is transformed by an adenovirus E1 region of a functional equivalent thereof. A preferred example of such a cell line is the PER C6™ cell line or equivalent thereof. In a particularly preferred embodiment, said cell is a CHO cell or a variant thereof. Preferably a variant that makes use of a Glutamine synthetase (GS) vector system for expression of an antibody.

The invention further provides a pharmaceutical composition comprising an antibody according to the invention. The pharmaceutical composition preferably comprises a preferably pharmaceutically acceptable excipient or carrier. In a preferred embodiment the pharmaceutical composition comprises 5-50 mM Histidine, 100-300 mM Trehalose, 0.1-03 g/L PolySorbate20 or a combination thereof. The pH is preferably set at pH=5.5-6.5. In a preferred embodiment the pharmaceutical composition comprises 25 mM Histidine, 220 mM Trehalose, 0.2 g/L PolySorbate20 or a combination thereof. The pH is preferably set at pH=5.5-6.5, most preferably at pH=6.

An antibody of the invention preferably further comprises a label, preferably a label for in vitro imaging. Such a label is typically not necessary for therapeutic applications. In for instance a diagnostic setting, a label can be helpful. For instance in visualizing target cells in the body. Various labels are suited and many are well known in the art. In a preferred embodiment, the label is a radioactive label for detection. In another preferred embodiment, the label is an infrared label. Preferably the infrared label is suited for in vivo imaging. Various infrared labels are available in the person skilled in the art. Preferred infrared labels are for instance, IRDye 800; IRDye 680RD; IRDye 680LT; IRDye 750; IRDye 700DX; IRDye 800RS IRDye 650, IRDye 700 phosphoramidite; IRDye 800 phosphoramidite (LI-COR USA; 4647 Superior Street; Lincoln, Nebraska).

The invention further provides a method for the treatment of a subject having an EGFR, ErbB-3 or EGFR/ErbB-3 positive tumor or at risk of having said tumor comprising administering to the subject an antibody or pharmaceutical composition according to the invention. Before start of said treatment, the method preferably comprises determining whether said subject has, or is at risk of, such EGFR, ErbB-3 or EGFR/ErbB-3 positive tumor. The invention further provides an antibody or pharmaceutical composition of the invention for use in the treatment of a subject having or at risk of having an EGFR, ErbB-3 or EGFR/ErbB-3 positive tumor.

To establish whether a tumor is positive for EGFR the skilled person can for instance determine the EGFR amplification and/or staining immune-histochemistry. At least 10% of the tumor cells in a biopt should be positive. The biopt can also contain 20%, 30% 40% 50% 60% 70% or more positive cells. To establish whether a tumor is positive for HER3 the skilled person can for instance determine the HER3 amplification and/or staining in immunohistochemistry. At least 10% tumor cells in a biopt should be positive. The biopt can also contain 20%, 30% 40% 50% 60% 70% or more positive cells.

The tumor is preferably an EGFR, ErbB-3 or EGFR/ErbB-3 positive cancer. Preferably said positive cancer is a breast cancer, such as early-stage breast cancer. However, the invention can be applied to a wide range of EGFR, ErbB-3 or EGFR/ErbB-3 positive cancers, like breast cancer, colon cancer, pancreatic cancer, gastric cancer, ovarian cancer, colorectal cancer, head- and neck cancer, lung cancer including non-small cell lung cancer, bladder cancer and the like. The subject is preferably a human subject. The subject is preferably a subject eligible for antibody therapy using an EGFR specific antibody such as cetuximab. In a preferred embodiment the subject comprises a tumor, preferably an EGFR/ErbB-3 positive cancer, preferably a tumor/cancer with an EGFR monoclonal antibody resistant phenotype.

The amount of antibody according to the invention to be administered to a patient is typically in the therapeutic window, meaning that a sufficient quantity is used for obtaining a therapeutic effect, while the amount does not exceed a threshold value leading to an unacceptable extent of side-effects. The lower the amount of antibody needed for obtaining a desired therapeutic effect, the larger the therapeutic window will typically be. An antibody according to the invention exerting sufficient therapeutic effects at low dosage is, therefore, preferred. The dosage can be in range of the dosing regime of cetuximab. The dosage can also be lower.

A bispecific antibody according to the invention preferably induces less skin toxicity as compared to cetuximab under otherwise similar conditions of course. A bispecific antibody according to the invention preferably produces less proinflammatory chemokines, preferably of CXCL14 as compared to cetuximab under otherwise similar conditions of course. A bispecific antibody according to the invention preferably induces less impairment of antimicrobial RNAses, preferably Rnase 7, as compared to cetuximab under otherwise similar conditions of course.

The present invention describes among others antibodies that target the EGFR and ErbB-3 receptors and result in potent proliferation inhibition of cancer cell lines in vitro and tumor growth inhibition in vivo. A diverse panel of human and Fab binding arms specific for either EGFR or ErbB-3 were identified. These were produced as bispecific antibodies by cloning them into complementary expression vectors that contain mutations in the CH3 region that drives hetero-dimerization of heavy chains. Many bispecific antibodies were produced at small scale and tested in binding and functional assays on cancer cell lines. Various bispecific antibodies were selected and tested in an orthotopic xenograft model using the BxPC3-fuel cell line. This cell line expresses both the EGFR and ErbB-3 receptors and is partially dependent on the presence of an EGFR ligand and an Erb8-3 ligand for growth. BxPC3 models are a robust and stringent screening model. An antibody of the invention, particularly a bispecific antibody of the invention can combine low toxicity profiles with high efficacy. An antibody of the invention can be useful in various types and lines of EGFR-targeted therapies. An antibody of the invention can have an increased therapeutic window when compared to an antibody that binds the same antigen(s) with both arms. A bispecific antibody of the invention can exhibit better growth inhibitory effects in vitro, in vivo or a combination thereof when compared to the MEHD7945A antibody.

Preferred embodiments of the invention provide uses of antibodies according to the invention under heregulin stress conditions. Heregulin is a growth factor that is involved in growth of ErbB-3 positive tumor cells. Typically, when the tumor cells express high levels of heregulin (referred to as heregulin stress), currently known therapies like trastuzumab, pertuzumab and lapatinib are no longer capable of inhibiting tumor growth. This phenomenon is called heregulin resistance. Surprisingly, however, an antibody according to the invention is also capable of counteracting growth of tumor cells that express high levels of heregulin. As used herein, an expression level of heregulin is considered high if a cell has a heregulin expression level that is at least 60%, preferably at least 70%, more preferably at least 80%, more preferably at least 85%, more preferably at least 90% or 95% of the heregulin expression level of BXPC3 or MCF7 cells. Heregulin expression levels are for instance measured using qPCR with tumor RNA (such as for instance described in Shames et al. PLOS ONE, February 2013, Vol.8, Issue 2, pp 1-10 and in Yonesaka et al., Sci. transl. Med., Vol.3, Issue 99 (2011); pp 1-11), or using protein detection methods, like for instance ELISA, preferably using blood, plasma or serum samples (such as for instance described in Yonesaka et al., Sci. transl. Med. Vol.3, Issue 99 (2010); pp 1-11).

Also provided is a method for counteracting the formation of a metastasis in a subject having a EGFR, ErbB-3 or EGFR/ErbB-3 positive tumor, wherein said EGFR, ErbB-3 or EGFR/ErbB-3 positive tumor has a heregulin expression level that is at least 60%, preferably at least 70%, more preferably at least 80%, more preferably at least 85%, more preferably at least 90% or 95% of the heregulin expression level of BXPC3 or MCF7 cells, comprising administering to the subject a bispecific antibody comprising a first antigen-binding site that binds EGFR and a second antigen-binding site that binds Erb B-3. Also provided is a bispecific antibody comprising a first antigen-binding site that binds EGFR and a second antigen-binding site that binds ErbB-3 for use in the treatment or prevention of focal adhesion of a EGFR, ErbB-3 or EGFR/ErbB-3 positive tumor cell, or for use in the treatment or prevention of the formation of metastases, wherein said EGFR, ErbB-3 or EGFR/ErbB-3 positive tumor cell has a heregulin expression level that is at least 60%, preferably at least 70%, more preferably at least 80%, more preferably at least 85%, more preferably at least 90% or 95% of the heregulin expression level of BXPC3 or MCF7 cells. Further provided is a use of a bispecific antibody according to the invention for the preparation of a medicament for the treatment or prevention of focal adhesion of a EGFR, ErbB-3 or EGFR/ErbB-3 positive tumor cell, or for the treatment or prevention of the formation of metastases, wherein said EGFR, ErbB-3 or EGFR/ErbB-3 positive tumor cell has a heregulin expression level that is at least 60%, preferably at least 70%, more preferably at least 80%, more preferably at least 85%, more preferably at least 90% or 95% of the heregulin expression level of BXPC3 or MCF7 cells. Said EGFR, ErbB-3 or EGFR/ErbB-3 positive tumor is preferably breast cancer, colon cancer, pancreatic cancer, gastric cancer, ovarian cancer, colorectal cancer, head- and neck cancer, lung cancer including non-small cell lung cancer, bladder cancer and the like. Most preferably, said tumor is breast cancer. Further provided is therefore a bispecific antibody according to the invention comprising a first antigen-binding site that binds EGFR and a second antigen-binding site that binds ErbB-3 for use in the treatment or prevention of focal adhesion, or the formation of metastases, of breast cancer, colon cancer, pancreatic cancer, gastric cancer, ovarian cancer, colorectal cancer, head- and neck cancer, lung cancer including non-small cell lung cancer, bladder cancer and the like, preferably breast cancer cells, wherein said cells have a heregulin expression level that is at least 60%, preferably at least 70%, more preferably at least 80%, more preferably at least 85%, more preferably at least 90% or 95% of the heregulin expression level of BXPC3 or MCF7 cells. Said antibody according to the present invention is typically capable of reducing a ligand-induced receptor function, preferably ligand induced growth, of ErbB3 on a ErbB2 and ErbB3 positive cell. Said antibody according to the invention preferably comprises an antigen-binding site that binds domain III of ErbB-3. The affinity (KD) of said ErbB-3 antigen-binding site for an ErbB-3 positive cell is preferably lower than or equal to 2.0 nM, more preferably lower than or equal to 1.39 nM, more preferably lower than or equal to 0.99 nM.

In one preferred embodiment, said antibody according to the invention preferably comprises an antigen-binding site that binds at least one amino acid of domain III of ErbB-3 selected from the group consisting of R426 and surface-exposed amino acid residues that are located within 11.2 Å from R426 in the native ErbB-3 protein.

One preferred embodiment provides a use of an antibody according to the invention for the preparation of a medicament for the treatment of an EGFR, ErbB-3 or EGFR/ErbB-3 positive tumor, wherein cells of said tumor have a heregulin expression level that is at least 60%, preferably at least 70%, more preferably at least 80%, more preferably at least 85%, more preferably at least 90% or 95% of the heregulin expression level of BXPC3 or MCF7 cells. Said EGFR, ErbB-3 or EGFR/ErbB-3 positive tumor is preferably breast cancer, gastric cancer, colorectal cancer, colon cancer, gastro-esophageal cancer, esophageal cancer, endometrial cancer, ovarian cancer, liver cancer, lung cancer including non-small cell lung cancer, clear cell sarcoma, salivary gland cancer, head and neck cancer, brain cancer, bladder cancer, pancreatic cancer, prostate cancer, kidney cancer, skin cancer, or melanoma. Most preferably, said tumor is breast cancer. Further provided is therefore an antibody according to the invention for use in the treatment of a subject having or at risk of having breast cancer, gastric cancer, colorectal cancer, colon cancer, gastro esophageal cancer, esophageal cancer, endometrial cancer, ovarian cancer, liver cancer, lung cancer including non-small cell lung cancer, clear cell sarcoma, salivary gland cancer, head and neck cancer, brain cancer, bladder cancer, pancreatic cancer, prostate cancer, kidney cancer, skin cancer, or melanoma, preferably breast cancer, wherein cells of said cancer have a heregulin expression level that is at least 60%, preferably at least 70%, more preferably at least 80%, more preferably at least 85%, more preferably at least 90% or 95% of the heregulin expression level of BXPC3 or MCF7 cells.

High heregulin levels are typically present during the formation of metastases (i.e. the migration, invasion, growth and/or differentiation of tumor cells or tumor initiating cells). Typically, tumor initiating cells are identified based on stem cell markers such as CD44. These processes can therefore barely be counteracted with currently known therapies like trastuzumab and pertuzumab. Since an antibody according to the invention is capable of counteracting growth and/or differentiation of tumor cells or tumor initiating cells that express high levels of heregulin, such antibody according to the invention is also particularly suitable for counteracting the formation of metastases. Further provided is therefore a method for counteracting the formation of a metastasis in a subject having a EGFR, ErbB-3 or EGFR/ErbB-3 positive tumor, wherein said EGFR, ErbB-3 or EGFR/ErbB-3 positive tumor cell has a heregulin expression level that is at least 60%, preferably at least 70%, more preferably at least 80%, more preferably at least 85%, more preferably at least 90% or 95% of the heregulin expression level of BXPC3 or MCF7 cells, comprising administering to the subject a bispecific antibody comprising a first antigen-binding site that binds EGFR and a second antigen-binding site that binds ErbB-3. Also provided is a bispecific antibody comprising a first antigen-binding site that binds EGFR and a second antigen-binding site that binds ErbB-3 for use in the treatment or prevention of the formation of metastases, wherein said EGFR, ErbB-3 or EGFR/ErbB-3 positive tumor cell has a heregulin expression level that is at least 60%, preferably at least 70%, more preferably at least 80%, more preferably at least 85%, more preferably at least 90% or 95% of the heregulin expression level of BXPC3 or MCF7 cells. Further provided is a use of a bispecific antibody according to the invention for the preparation of a medicament for the treatment or prevention of the formation of metastases, wherein said EGFR, ErbB-3 or EGFR/ErbB-3 positive tumor cell has a heregulin expression level that is at least 60%, preferably at least 70%, more preferably at least 80%, more preferably at least 85%, more preferably at least 90% of 95% of the heregulin expression level of BXPC3 or MCF7 cells. Said EGFR, ErbB-3 or EGFR/ErbB-3 positive tumor is preferably breast cancer, gastric cancer, colorectal cancer, colon cancer, gastro-esophageal cancer, esophageal cancer, endometrial cancer, ovarian cancer, liver cancer, lung cancer including non-small cell lung cancer, clear cell sarcoma, salivary gland cancer, head and neck cancer, brain cancer, bladder cancer, pancreatic cancer, prostate cancer, kidney cancer, skin cancer, or melanoma. Most preferably, said tumor is breast cancer. Further provided is therefore a bispecific antibody according to the invention comprising a first antigen-binding site that binds EGFR and a second antigen binding site that binds ErbB-3 for use in the treatment or prevention of the formation of metastases of breast cancer, gastric cancer, colorectal cancer, colon cancer, gastro esophageal cancer, esophageal cancer, endometrial cancer, ovarian cancer, liver cancer, lung cancer including non-small cell lung cancer, clear cell sarcoma, salivary gland cancer, head and neck cancer, brain cancer, bladder cancer, pancreatic cancer, prostate cancer, kidney cancer, skin cancer, or melanoma cells, preferably breast cancer cells, wherein said cells have a heregulin expression level that is at least 60%, preferably at least 70%, more preferably at least 80%, more preferably at least 85%, more preferably at least 90% or 95% of the heregulin expression level of BXPC3 or MCF7 cells. Said antibody according to the present invention is typically capable of reducing a ligand-induced receptor function, preferably ligand induced growth, of ErbB-3 on a EGFR and ErbB-3 positive cell. Said antibody according to the invention preferably comprises a first antigen-binding site that binds domain I of EGFR and a second antigen-binding site that binds domain III of ErbB-3. The affinity of said second antigen-binding site for an ErbB-3 positive cell is preferably lower than or equal to 2.0 nM, more preferably lower than or equal to 1.39 nM, more preferably lower than or equal to 0.99 nM.

In one embodiment, said bispecific antibody is for use in the treatment of a subject under heregulin stress conditions, as explained in more detail herein above.

Antibodies of the invention can be produced at levels >50 mg/L after transient transfection in suspension 293F cells. The bispecific antibodies can be purified to greater than 98% purity with yields >70%. Analytical characterization studies show bispecific IgG1 antibody profiles that are comparable to bivalent monospecific IgG1. In terms of functional activity a bispecific antibody of the invention can demonstrate superior potency compared to MEHD7945A in vitro and in vivo.

For the purpose of clarity and a concise description features are described herein as part of the same or separate embodiments, however, it will be appreciated that the scope of the invention may include embodiments having combinations of all or some of the features described.

### BRIEF DESCRIPTION OF THE DRAWINGS

FIG. 1: Annotated sequence of the chimeric cynomolgus-human EGFR encoding construct. Restriction sites used for re-cloning (Nhe1-Not1) are indicated in capitals. The start of the mature peptide is underlined and indicated in bold. The trans-membrane region is in bold and italics.

FIG. 2: Amino acid sequences of the extra-cellular domain (ECD) of EGFR ‘swap-domain variants’: the human EGFR ECD was used as backbone and specific domains were swapped for the human HER3 sequence. HER3-derived sequences are indicated. These sequences were cloned in frame with a c-Myc derived epitope tag and the trans-membrane region of the platelet-derived growth factor receptor (PDGFR).

FIG. 3: Epitope mapping of anti-EGFR Fab's (MF number; selected from the ‘immune’ phage libraries) expressed on phage by competition for binding with known, literature-derived antibodies. Representative OD values obtained after testing of phage binding to immobilized EGFR in ELISA in the presence (or absence) of the indicated antibodies ND: not determined. The domain specificity of the control antibodies is indicated.

FIG. 4: example of epitope mapping of selected anti-EGFR common light chain (cLC) IgG (PG numbers) by FACS staining using EGFR swap domain mutants. Antibodies were tested for binding to CHO cells stably expressing the indicated EGFR swap mutants (domains in the wtEGFR sequence that were swapped for the corresponding domain of HER3). Light line: negative control (irrelevant antibody) staining; dark line: antibody staining. vIII: EGFR variant III, lacking domain I and most of domain II). Ctr: negative control staining (irrelevant primary antibody added). The determined domain specificity is indicated on the right of the Figure.

FIG. 5: example of the functionality of anti-EGFR cLC IgG (PG numbers) in inhibiting the EGF-induced death of A431 cells.

Y axis (counts) shows the fluorescence readout of the assay, reminiscent of the number of metabolically active cells, as a function of the concentration of antibody used (X-axis). Antibodies that show functional EGFR blocking activity dose-dependently inhibit the EGF-induced cell death and therefore show enhanced growth of the cells with increasing antibody concentration. The clinically used antibody cetuximab was used in all experiments as an internal standard (diamonds).

FIG. 6: overview of the screening of anti-EGFRxHER3 bispecifics, compared to the activity of the MEHD7945A antibody. Every bar represents the activity of a bispecific anti-EGFRxHER3 antibody in the BxPC-3 ligand-independent (light bars) and ligand-driven assay (dark bars). The average activity of the MEHD7945A antibody is indicated by a light (upper) and dark (lower) line for the independent and dependent assay respectively. Growth was normalized to untreated controls. Over 800 bispecific anti-EGFRxHER3 antibodies were screened.

FIG. 7: IC50 determination for BxPC-3 cell proliferation inhibition of the two best performing bispecifics (PB numbers), compared to MEHD7945A. The Y-axis represents the percentage of growth, normalised to the control situation (i.e. uninhibited growth in the presence of both ligands) and the X-axis the antibody concentration used. IC50 values are indicated.

FIG. 8: therapeutic effect of bispecific antibody (PB numbers) treatment on the growth of BxPC-3 cells orthotopically implanted. The Y-axis shows the bioluminescence (BLI) readout (reminiscent of the number of live tumor cells) after injection of luciferin in the mice as a function of time (X-axis). The MEHD7945A antibody was used as reference and an irrelevant (anti-RSV) antibody as a negative control antibody (squares). For the sake of clarity, error bars have been omitted from the Figure.

FIG. 9: therapeutic effect of the lead bispecific PB4522 in comparison with that of MEHD7945A at 0.3 mg/kg dose. Y axis shows the in vivo bioluminescence (BLI), reminiscent of the number of live tumor cells as a function of time (X-axis). NC: negative control antibody (anti-RSV, 30 mg/kg dose). For the sake of clarity, error bars have been omitted from the Figure.

FIG. 10: tumor weights measured ex vivo at day 38 of the study of tumors isolated from mice treated with either MEHD7945A (group G4) or PB4522 (group G7) at 0.3 mg/kg. The left panel shows the weight of the tumor of every individual mouse and the right panel shows the average weight of the 7 mice that were treated. ** p<0.05.

FIGS. 11A-11D: Nucleic acid and amino acid sequences of VH-chains, common light chain and heavy chains of antibodies of the invention. FIG. 11A depicts heavy chain variable regions that bind EGFR. Where in this Figure a leader sequence is indicated this is not part of the VH chain or antibody, but is typically cleaved of during processing of the protein in the cell that produces the protein. The VH chain sequence of a heavy chain indicated with the capitals MG followed by a number in the text is indicated herein by the same number preceded with the letters VH. FIG. 11B further specifies the amino acid sequences of heavy chain variable region sequences of an erbB-3 binding antibodies MF6055-MF6074. The variable heavy chain sequences are variants of heavy chain variable region MF3178. Dots indicate the same amino acid as in MF3178 at that position. The CDR regions are separated by a space and indicated in bold. FIG. 11C depicts light chain variable region sequences. FIG. 11D depicts heavy chain sequences for EGFR and erbB-3 binding.”

FIG. 12: CXCL14 and Rnase7 expression in primary keratinocytes stimulated with different IgGs. Expression was measured by Q-PCR in the presence of two antibody concentrations.

FIG. 13: ADCC activity of afucosylated PB4522 compared to MEHD7945A and cetuximab on high EGFR (A431) and intermediate EGFR (BxPC3 and A549) expressing cells. ‘Ctrl Ab’ is an IgG directed against Tetanus Toxoid

FIG. 14: Titration curves of HER3 monoclonal antibodies in the HRG dependent N87 assay. PG6058, PG6061 and PG6065 are variants of PG3178. PG1337 is a negative control specific for tetanus toxoid. Data were normalized to basal proliferation with ligand present on each plate.

FIG. 15: CIEX-HPLC profiles of HER3 monoclonal antibodies. PG6058, PG6061 and PG6065 are variants of PG3178. The calculated iso-electric point (pI) of the VH region and the retention time (tR) of the main peak are given for each antibody.

FIGS. 16A-16C: FIG. 16A HER3 crystal structure (PDB #4P59) showing epitope residue Arg 426 in gray spheres and all surface exposed residues within an 11.2 Å radius from Arg 426 in black spheres, FIG. 16B Solvent exposed surface of epitope region with Arg 426 and distant residues shown in gray and all surface exposed residues within a 11.2 Å radius from Arg 426 shown in black, FIG. 16C Residues in the epitope region Arg 426 in light gray and surrounding residues (all labeled) in dark gray. Figures and analyses were made with Yasara (www.yasara.org).

FIG. 17: Critical residues for PG3178 binding represented in the HER3 crystal structure. Critical residues identified for PG3178 binding are represented as black spheres on the HER3 crystal structure (PDB ID #4P59).

FIG. 18: Confirmation of R426 as a critical binding residue for PG3175 to HER3. Two anti-HER3 antibodies were included as control antibodies. Binding was determined in a FACS titration and binding is expressed as AUC in comparison to binding to WT HER3.

### EXAMPLES

### Cell Lines:

BxPC-3-luc2 (Perkin Elmer 125058), CHO-K1 (DSMZ ACC110), 293F (Invitrogen R790-07), A549 ATCC® CCL-185™ Homo sapiens lung Carcinoma, BxPC-3 ATCC® CRL-1687™ Homo sapiens pancreas adenocarcinoma, and A431 cells DSMZ ACC 91 were purchased and routinely maintained in growth media supplemented with 10% fetal heat inactivated bovine serum (FBS). 293F Freestyle cells were obtained from Invitrogen and routinely maintained in 293 FreeStyle medium. BxPc-3-luc2 (Bioware® Ultra Light Producing) cell line is a luciferase expressing cell line which was stably transfected with firefly luciferase gene (luc2). The cell line was established by transducing lentivirus (pGL4 luc2) containing luciferase 2 gene under the control of human ubiquitin C promoter. The cell line is commonly referred to as BxPc-3-luc2, it is a human cell pancreas adenocarcinoma cell line derived from BxPC-3 ATCC (CRL-1687™). Bioluminescence In Vitro: Approximately 370 photons/sec/cell. Exact number will vary depending on imaging and culturing conditions.

Cloning of the cDNA Encoding Cynomolgus EGFR and EGFR Swap-Domain Constructs

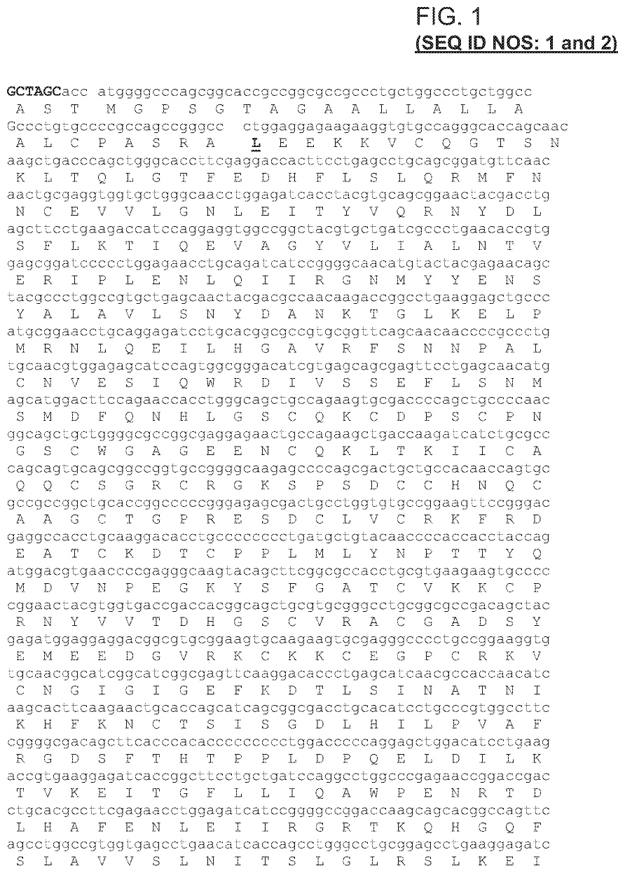

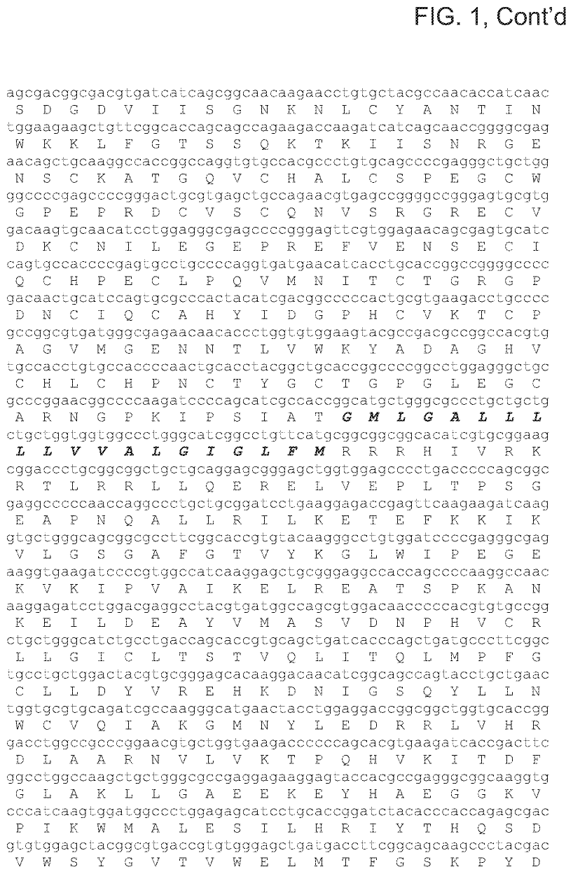

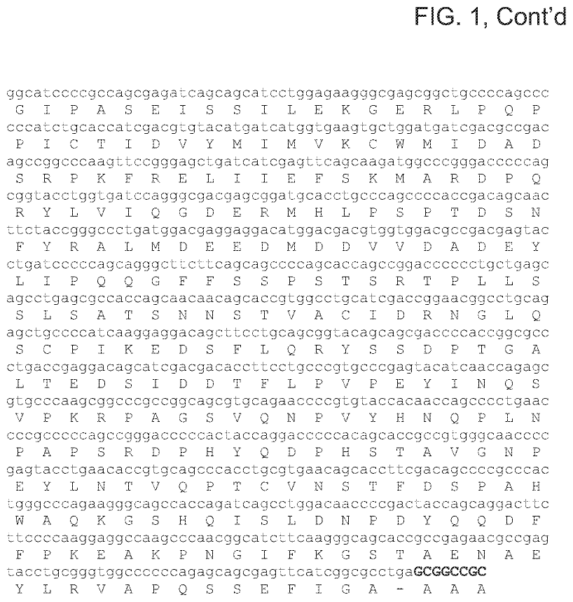

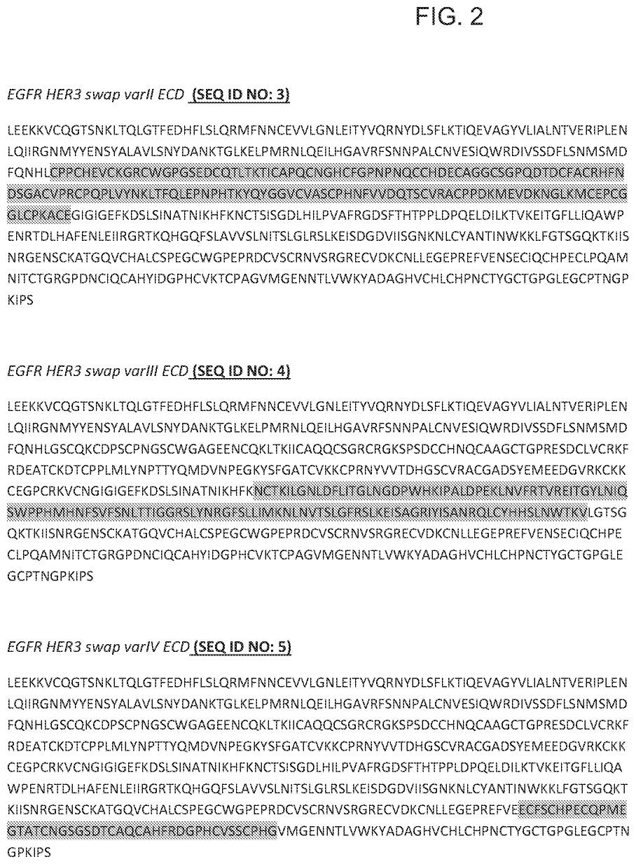

A construct encoding a chimeric receptor comprising the extra cellular domain (ECD) of cynomolgus EGFR fused to the human trans-membrane domain and intra-cellular tail was copied from a published patent application by Micromet (US 2010/0183615 A1). The sequence is depicted in FIG. 1. This cDNA was synthetically made by GeneArt and was cloned in the vector pMK-RQ, flanked by the restriction sites Nhe1 and Not1. The cDNA was liberated from this vector using these two enzymes and then cloned into the mammalian expression vector pCDNA3.1. The cDNA was fully sequenced to make sure it matched the designed construct and no mutations were found. For epitope mapping purposes, constructs were generated in which the different domains in the extra-cellular domain (ECD) of EGFR (L1, CR1, L2 and CR2, also named domains I-IV: [1]) were swapped for the corresponding domains of HER3: FIG. 2. These were then cloned into an expression vector for transient expression in (antigen-negative) CHO cells, in frame with an extra cellular cMyc-derived epitope tag and the trans-membrane region of the platelet-derived growth factor receptor. Constructs were designed, synthesised by GeneArt and subsequently cloned into pDisplay as Sti1-Sal1 fragments. All constructs were sequenced, shown to be correct and tested for expression. Only the domain I swap variant (HER3 domain I with HER1 domains II-IV) was shown not to give rise to detectable amounts of protein on the surface of transfected cells (not shown).

The reference numbers used in the Examples refer to the numbering of the references in the list headed “References cited in the Examples”.

Immunisations of MeMo with rhEGFR-Fc Fusion Protein and A431 Cells Over-Expressing the Antigen

Mice that are transgenic for a rearranged human VL and a divers set of unrearranged human VH, DH and JH gene segments operably linked to murine constant region genes (MeMo®; see also WO2009/157771) were immunized with EGFR-Fc protein (R&D Systems, cat nr. 344-ER) emulsified with TitermaxGold adjuvant (TMG, Sigma Aldrich, cat. nr. T2684) at an interval of 14 days. MeMo produces common light chain (cLC) antibodies upon exposure to an antigen, i.e.: all antibodies coming out of Memo carry essentially the same light chain yet are diversified in their heavy chains. At day 0 and 14 mice were vaccinated subcutaneously (s.c.) to minimize the TMG induced discomfort of the mice. At all later time points mice were immunized via the intra-peritoneal (i.p.) route to insure the antigen is efficiently taken up and presented in the spleen. At day 35, the anti-EGFR serum titer was determined by FACS using the EGFR over-expressing cell line A431. Mice that developed at day 35 a serum titer >1/1,000 received a final i.p. boost with EGFR-Fc protein dissolved in PBS at day 42 followed by collection of spleen and lymph node three days later. Mice with too low EGFR serum titer at day 35 received an additional i.p. boost of EGFR-Fc in TMG at day 49.

Subsequently, serum titers were determined at day 56 by ELISA. Those mice that at day 56 had a serum titer above the acceptance criteria received a final i.p. boost with EGFR-Fc protein dissolved in PBS at day 63 followed by collection of spleen and lymph node. Mice were injected with 20 μg EGFR-Fc protein dissolved in 125 μl TMG or 200 μl PBS.

Two MeMo mice immunized with EGFR-Fc received also a boost with A431 cells. This was because these mice had developed a low a-EGFR-Fc IgG serum titer that was primarily directed against the Fc-tail. To specifically boost the EGFR response were these mice boosted with a single i.p. injection of 2E+6 A431 cells in 200 μl PBS at day 49.

All mice were shown to have mounted a significant response directed to the extracellular domain of EGFR, as witnessed by their reactivity with CHO cells stably expressing full length human EGFR and with A431 cells over-expressing EGFR in FACS.

### Generation of ‘Immune’ Phage Antibody Libraries From Immunised Mice

After immunisation, RNA was extracted from lymphoid tissues (lymph nodes, spleen) and the polyclonal pool of VH-encoding cDNA's was amplified using VH family specific primers as previously described (e.g. EP 2604625). Material of two mice was pooled for library construction. The resulting PCR products were then cut with the restriction enzymes Sti1 and BstEII and cloned in frame with the bacteriophage gene III for display on filamentous bacteriophage, essentially as described in [2], only the phagemid vector already contained the Vκ1-39 germline VL gene. Phage library ML1155 was generated from the two mice with the highest α-EGFR serum titer (mice E094#12 and E094#18). Library ML1156 was generated from the two mice that received the additional A431 boost (mice E094#14 and E094#16). Characteristics of the libraries are depicted in Table 1.

### Phage Selections for EGFR Binding Clones

The (Fc-) immune-fusion of the ecto-domain of EGFR (R&D systems) was diluted in PBS (to 5 μg/ml and in two-fold dilutions thereof) and coated to the wells of Maxisorp immune-plates. Phage were then panned for binding as described [3]. Selection of Fc-reactive clones was avoided by stringent counter-selection using (10 μg of) soluble IgG during the incubation of phage with coated antigen. To direct the selections on EGFR towards the ligand binding (L1) domain I, competition with an excess of the EGFR variant III (vIII) immune-fusion was also performed. In order to obtain cLC antibodies with functionality in inhibiting the receptor, phage selections on immobilised antigens were performed in combination with epitope-specific elution [4] using the either the ligand (EGF), or antibodies copied from literature (Matuzumab, Cetuximab). Elutions of EGFR-reactive phage were also performed using the domain I specific antibody ICR10 (Abcam, nr. ab231) and the domain II-specific antibody EGFR.1 (Thermo Scientific, nr. MS-311). As A431 cells [5] have been shown to carry an amplification of the EGFR gene [6] and therefore express high numbers of the EGFR, these were used for selection of EGFR-reactive phage.

Output phage titers were determined and for every selection where the output phage titer was 1.5 times above background (selection on a non antigen-coated well), 48 different clones were picked and phage were screened for binding to the respective antigens in ELISA. All clones were also tested for binding to human IgG in ELISA to identify binders to the Fc-portion of the immune-fusions used for selection. A high percentage of positive clones (defined as clones recognising the respective immune-fusions in ELISA, but not being IgG-reactive) was found in all selection outputs (up to 0.625 μg/ml) of coated immune-fusion. Clones that scored positive in phage ELISA on EGFR-Fc but not on hIgG were then sequenced and sequences were compared. Sequences were analysed as previously described (EP 2604625) and grouped on the basis of their VH gene segment usage and heavy chain CDR3 (HCDR3) sequence. An antibody cluster was defined as all sequences using the same germline VH segment having an HCDR3 with the same length and over 70% sequence identity in that HCDR3. In total, 17 antibody clusters were identified from immunised MeMo mice.

Using large synthetic cLC repertoires (synthesised in house) and selections on rhEGFR-Fc (as described above) many EGFR-specific clones were isolated, of which only one (MF3370) was later shown to be able to inhibit EGF-induced A431 cell death. This clone was therefore also analysed in more detail and used in subsequent EGFRxHER3 screening assays.

### Testing Selected Anti-EGFR Phage Antibodies for Competitive Binding With Control Antibodies

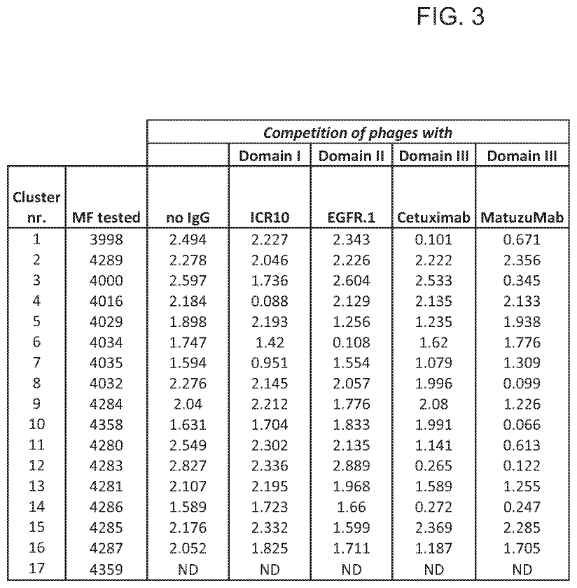

To delineate the epitope recognized by representative anti-EGFR Fabs, they were tested (as Fabs expressed on phage: named ‘MF’) for binding to EGFR in the presence of an excess of control, literature-derived IgG [7]. Control IgG used for these competition experiments are listed in Table 2. When phage expressing the selected Fabs were tested for binding to the antigen in ELISA in the presence of an excess of these control antibodies, several were found to be competed for binding by one or more of the control antibodies: FIG. 3. These results show the anti-EGFR panel to be diverse in epitope recognition.

### Re-Cloning of Selected Anti-EGFR cLC Fab's, Expression and Purification of cLC IgG1

EGFR specific phage clones (termed ‘MF’) were re-cloned as mono-specific, bivalent IgG (termed ‘PG’) by re-cloning the VH-encoding gene fragment (as Sti1-BstEII fragment) in an expression vector (named ‘MG’) for the transient expression of IgG in 293F cells. After production by transient transfection of 293F cells, secreted IgG (termed ‘PG’) was purified from the culture supernatant by prot. A affinity chromatography using standardised procedures.

### Testing of Anti-EGFR Antibodies for Their Cross-Reactivity With Cynomolgus EGFR and Mouse EGFR

To test whether anti-EGFR cLC IgG were reactive with cynomolgus EGFR, the constructs encoding full-length human EGFR, as well as the newly synthesized expression construct encoding the chimeric cynomolgus/human EGFR (FIG. 1) were both transfected in (antigen negative) CHO cells and cells were then stained with the anti-EGFR cLC IgG (PG) at 5 μg/ml and finally analysed by FACS. As a positive control for the staining, the clinically used antibody cetuximab was used, as this antibody is known to cross-react with cynomolgus EGFR [8]. The construct encoding the chimeric cyno-human receptor gave rise to high levels of the chimeric construct being expressed on the surface of transfected cells (as judged by the staining using cetuximab). In addition, all cLC IgG were shown to be reactive with cynomolgus EGFR, as the staining of cells expressing human EGFR was virtually indistinguishable from that of cells expressing the chimeric receptor.

To test anti-EGFR cLC IgG for their cross-reactivity with murine EGFR, an ELISA was performed. Purified protein, composed of the mouse EGFR ECD, fused to human IgG1 Fc was bought from Sino Biologicals (cat. nr. 51091-M02H) and a serial 2-fold dilution of this antigen was used to coat wells of an ELISA plate, starting at 5 μg/ml. Binding of the anti-EGFR cLC IgG to this antigen was then tested at a fixed concentration of 5 μg/ml. As a positive control for the immuno-reactivity of the antibodies, the same ELISA setup was performed using the human EGFR ECD-Fc fusion protein as antigen (R&D systems). From all 17 antibody clusters as described above, at least one representative antibody was tested. In addition, clone MF3370 from the synthetic library was also tested as an IgG (PG3370). All but one of the cLC antibodies were shown not to recognise mouse EGFR, as they failed to react with the fusion protein in ELISA. However, antibody PG3370 was shown to recognise murine EGFR, as well as human EGFR with similar affinity (data not shown): Table 3 summarises the data.

### Testing the Domain Specificity of Anti-EGFR Antibodies Using EGFR Swap-Domain Constructs in FACS

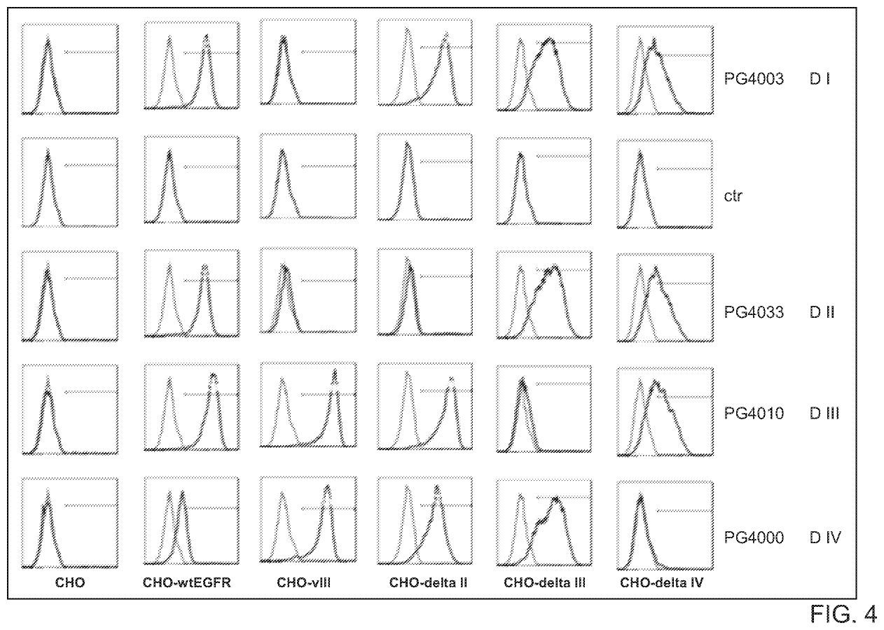

To unequivocally demonstrate the domain-specificity of the anti-EGFR cLC IgG, they were tested for binding to CHO cells stably expressing ‘swap-domain’ constructs: i.e. CHO cells expressing mutants of EGFR in which specific domains had been replaced for the corresponding domains of Her3 in FACS (FIG. 2). FIG. 4 gives an example of the FACS data obtained; data are summarized in Table 3.

### Testing Anti-EGFR Antibodies for Their Effect on Ligand-Induced Signalling

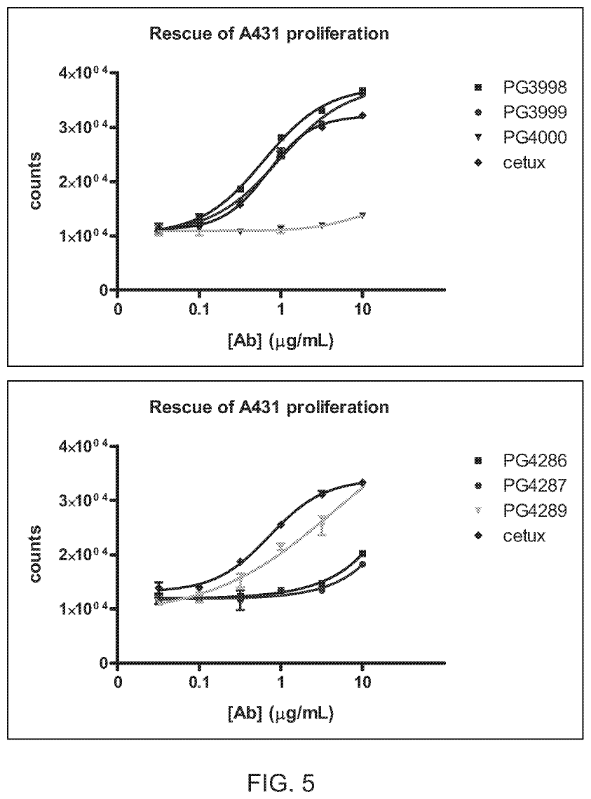

To test the selected anti-EGFR cLC IgG for their effects on EGF-induced signalling, they were tested for their ability to prevent the EGF-induced cell death of A431 cells. In brief, high (10 nM) concentrations of EGF induce (apoptotic) cell death in A431 cells [9]. This effect can be dose-dependently reverted by the addition of ligand-blocking anti-EGFR antibodies, such as cetuximab (the murine 225 antibody is the mouse equivalent of cetuximab: [9]) Antibodies were tested in this assay for their effect on receptor inhibition in a serial semi-log dilution from 10 μg/ml onwards. In every assay, the EGFR-blocking and clinically used antibody cetuximab was included as positive control. Anti-EGFR antibodies were found to have varying potencies in inhibiting EGF-induced cell death: some were more potent than cetuximab in rescuing the EGF-induced effect (e.g. PG3998: FIG. 5), some were less potent (e.g. PG4289, FIG. 5) and some had very little to no activity (e.g. PG4000, FIG. 5). Table 3 shows the activity of cLC antibodies directed to EGFR in inhibiting the receptor, compared to that of cetuximab.

### Screening of Bispecific Anti-EGFRxHER3 Antibodies for Their Capacity to Inhibit BxPC-3 Cell Proliferation

VH-encoding cDNA fragments from the EGFR and HER3 antibody panel were re-cloned into vectors encoding charge-engineered CH3 domains that forced the generation of bispecific antibodies (Gunasekaran et al., JBC 2010; PCT/NL2013/050294) after transient transfection into 293F cells (termed ‘PB’ for bispecific protein). Three different strategies were used in combining EGFR and HER3 arms in bispecific IgG format: I) bispecifics of which both parental antibodies had proven ligand blocking activity for the receptor in respective cellular assays (the A431 assay for EGFR and the MCF-7 assay for Her3) resulting in 120 unique bispecific antibodies (Table 4); II) bispecifics of which only one of the arms (either the anti-Her3 or the anti-EGFR) had functionality in the above-mentioned assays (a total of 440 unique bispecific antibodies, not shown) and III) bispecifics of which both Fab arms had no (or almost no) functionality in these assays (a total of 320 unique bispecific antibodies, not shown). In total, 880 unique bispecific antibodies were tested for their ability to inhibit the growth of BxPC-3 cells.

All 880 bispecific antibodies were produced by co-transfection and transient co-expression in 293F cells; IgG was purified from the culture supernatant and the buffer in which the protein was kept was changed to PBS according to standardised procedures. Purified protein was quantified using Octet analysis. Bispecifics were tested at two concentrations (1 μg/ml and 100 ng/ml) in a ligand-(EGF- and NRG-) dependent assay (addition of 100 ng/ml of EGF, next to 10 ng/ml of NRG), as well as in a ligand-independent assay (no ligand added).

First, antibodies were diluted in chemically defined starvation medium (CDS: RPMI1640 medium, containing 80 U penicillin and 80 μg of streptomycin per ml, 0.05% (w/v) BSA and 10 μg/ml holo-transferrin) and 50 μl of diluted antibody was added to the wells of a 96 wells black well clear bottom plate (Costar). Ligand was added (50 μl per well of a stock solution containing 40 ng/ml NRG and 400 ng/ml of EGF, diluted in CDS: R&D systems, cat. nr. 396-HB and 236-EG). In case of the ligand-independent assay, no ligand was added, instead only 50 μl of CDS. BxPC-3 cells were trypsinised, harvested and counted and 8000 cells in 100 μl of CDS were added to each well of the plate. Plates were left for an hour at rt before being put in a container inside a 37° C. cell culture incubator for three days. On the fourth day, Alamar blue (Invitrogen, #DAL1100) was added (20 μl per well) and the fluorescence was measured after 6 hours of incubation (at 37° C.) with Alamar blue using 560 nm excitation and 590 nm readout on a Biotek Synergy 2 Multi-mode microplate reader. Fluorescence values were normalised to uninhibited growth (no antibody, but both ligands added).

### IC50 Determination

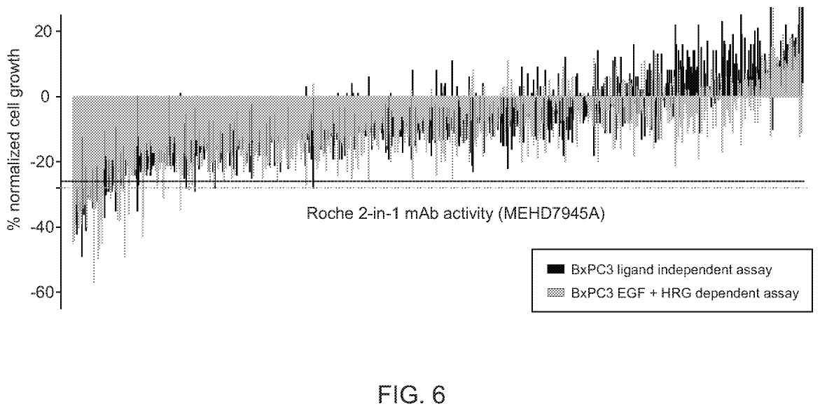

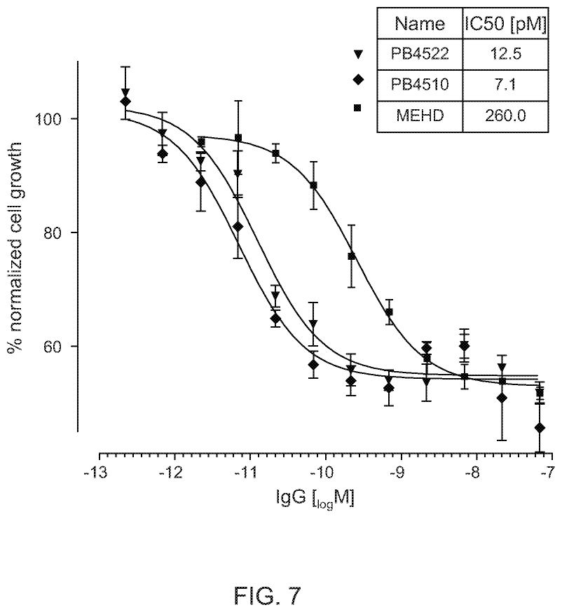

An overview of the screening data of the 880 bispecific antibodies is given in FIG. 6. MEHD7945A served as a benchmark antibody; the upper dotted line in FIG. 5 represents the average activity observed in the ligand-independent assay and the lower dotted line the average activity of that antibody observed in the ligand-dependent assay. Twenty bispecifics that had a potency in both assays that was at least as good as that of the MEHD7945A antibody were then re-produced, re-purified and tested again in the ligand-driven BxPC-3 proliferation assays to determine IC50 values. Antibodies were serially diluted in CDS from 10 μg/ml downward and 50 μl of antibody solution was added per well. Ligands and cells were added as described above and cells were incubated for three days before the addition of Alamar Blue and fluorescence readout. Again, values were normalised to uninhibited growth and IC50 values were calculated using GraphPad Prism software (non-linear curve fitting). The six best performing bispecifics were selected based on their IC50 being lower than that of the comparator antibody MEHD7945A in both assays. FIG. 7 shows the IC50 determination of two of these best performing antibodies; Table 6 summarises the data for these six bispecifics, as compared to the activity of a mixture of each of the parental monoclonal antibodies (see below).

### Testing Anti-EGFRxHER3 Bispecifics for Their Effect on the Growth of BxPC-3 Tumours Orthotopically Implanted

CB17 SCID female mice, 8-10 weeks old at the beginning of the study were engrafted orthotopically in the pancreas with 1×10⁶ tumor cells in 20 μl. Therefore mice were anesthetized and laid on the right side to expose the left side and a 0.5 cm incision is made on the left flank region. The pancreas and spleen were exteriorized and 1×10⁶ tumor cells in 20 μl were injected into the sub-capsulary space of the pancreas tail. One week after implantation, bioluminescence (BLI) data were generated. For BLI imaging (once or twice weekly) left side view, all mice received 15 minutes prior to the imaging all of the mice receive i.p. injections of 150 mg/kg Luciferin (D-Luciferin-EF Potassium Salt, Cat. #E6552, Promega). Outlier animals—based on BLI/tumor volume—were removed and the mice were randomly distributed into groups of 7 mice each. On experimental day 8, the treatment was started. The animals in the antibody treatment group were dosed weekly for 3 consecutive weeks (days 0, 7, 14 and 21) with 30 mg/kg of antibody. At day 0 of the treatment the animals receive twice the loading dose, i.e. 60 mg/kg of antibody. The final imaging was carried out at day 31.

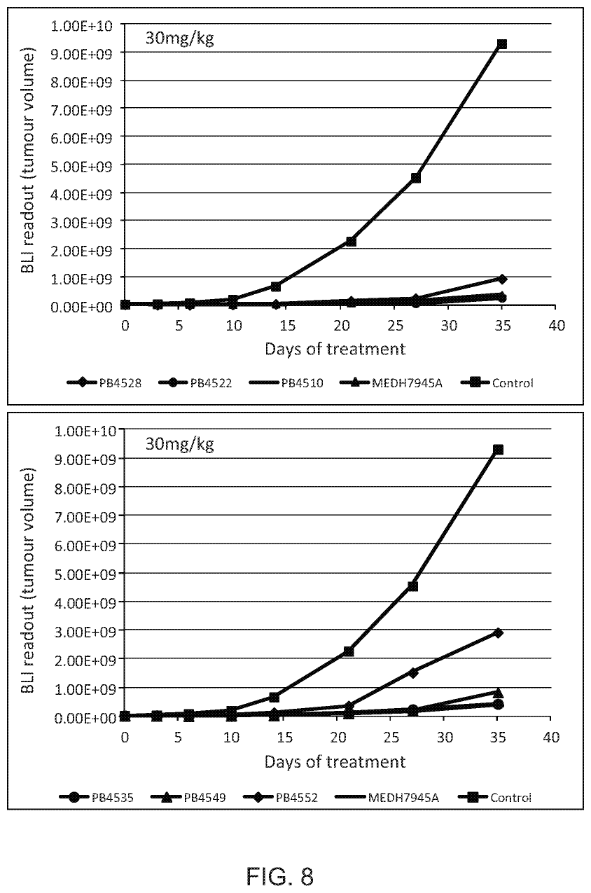

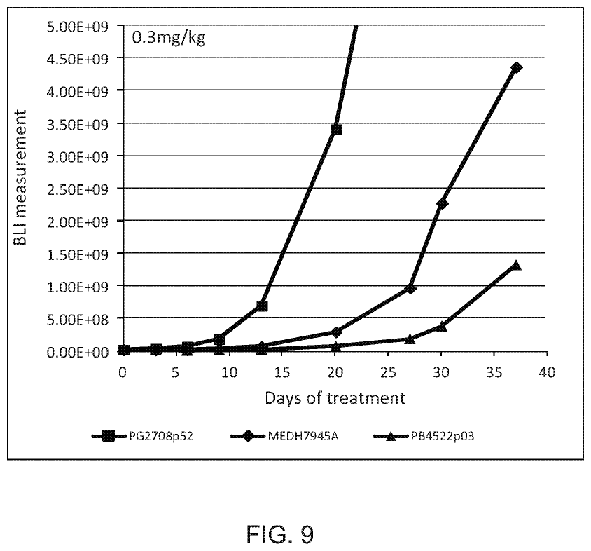

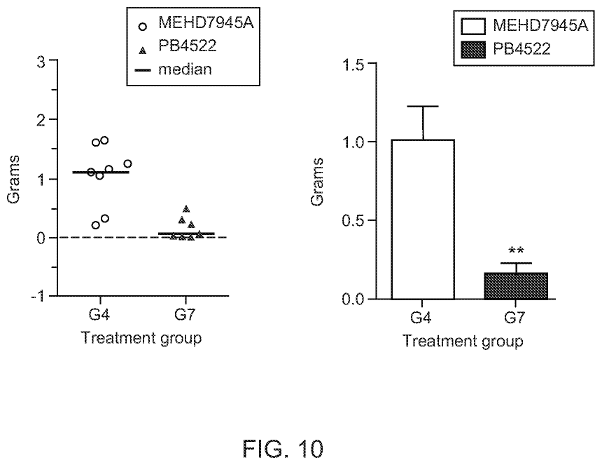

All six bispecifics were shown to significantly decrease BxPC-3 tumour outgrowth in the model (p<0.001) (FIG. 8). However, there was no significant difference between the therapeutic effect of the comparator antibody MEHD7945A and two of these six bispecifics, PB4522 and PB4510 (data not shown). Therefore, a dose escalation study was performed with one of the bispecific antibodies, PB4522, and the comparator antibody MEHD7945A. Using the exact same in vivo model and dosing schedule, different groups of mice were treated with a decreasing antibody dose, lowered from 30 mg/kg to 3 and finally 0.3 mg/kg. FIG. 9 shows the data for the groups treated with 0.3 mg/kg. There was no significant difference in therapeutic effect between all groups treated with either MEHD7945A or PB4522 at 30 and 3 mg/kg (data not shown). However, there was a significant difference in therapeutic effect between treatment with MEHD7945A and PB4522 at the 0.3 mg/kg dose, the latter being more potent in tumour growth reduction. After mice were taken out of the study, the weight of all their tumours was determined ex vivo. FIG. 10 shows that the average weight of tumours taken from mice treated with PB4522 (at 0.3 mg/kg) was significantly lower (P=0.007, unpaired T-Test) than that of mice treated with MEHD7945A (at 0.3 mg/kg).

Comparison of the Potency of Different Antibody Formats in BxPC-3 cell Proliferation Inhibition

To compare the potency of different antibody formats in cell proliferation inhibition, purified bispecific anti-EGFRxHER3 antibodies were tested for their ability to inhibit BxPC-3 cell proliferation in comparison with an equimolar mix of the parental antibodies. In every assay plate, MEHD7945A was used as positive control to be able to compare the IC50 of the antibody being tested directly with the ‘two-in-one’ MEHD7945A. Titrations were performed starting from 10 μg/ml and in 6 ten-fold dilutions. These serial dilutions of antibodies were tested in duplicate over the whole concentration range. From the obtained sigmoidal curves, IC50 values were calculated using the Graph Pad Prism software. Table 6 summarises the data. As can be seen in Table 6, in all cases the bispecific format was more potent then the mix of the two parental antibodies in inhibiting the proliferation of BxPC-3 tumour cells and this format was therefore the preferred format for co-targeting of EGFR and HER3. When the IC50 value for proliferation inhibition of same bispecific was measured several times, slightly different values were obtained in the different assays. However, these differences were considered to be unavoidable small experimental variations.

### Keratinocyte Assay

EGFR blockade has been demonstrated to affect chemokine expression in keratinocytes (Pastore, Mascia et al. 2005). Recent analyses of EGFR-Inhibitor (EGFRI) skin toxicities show that the early inflammatory infiltrate of the rash is dominated by dendritic cells, macrophages, granulocytes, mast cells and T-cells. EGFR inhibition induces the expression of chemokines (CCL2, CCL5, CCL27, CXCL14) in epidermal keratinocytes, while the production of antimicrobial peptides and skin barrier proteins such as Rnase 7 is impaired. The skin toxicity observed in vivo could be translated in vitro using a primary keratinocyte system in combination with Q-PCR analysis. (Lichtenberger, Gerber et al., Science Translational Medicine, 2013). The effect of PB4522 in comparison to MEHD7945A and cetuximab was tested at 10 and 100 nM concentrations on human primary epidermal keratinocytes. Human primary epidermal keratinocytes were isolated and seeded at a density of 100.000 cells per well in 6 well plates in SFM cell growth medium (Invitrogen) at 37° C., 5% CO2. Cells were treated in the absence or presence of 10 ng/ml TNF-α (AbD Serotec, Kidlington, UK) and 5 ng/ml IL-1β (R&D Systems, Inc., Minneapolis, MN) for 24 H. Duplicates of each condition were prepared. Twenty-four hours later cells were harvested and RNA was extracted from cells by using the TRIzol® Reagent. cDNA was synthesized from different messenger RNA (mRNA) templates using reverse transcriptase enzyme Superscript II (Invitrogen, Carlsbad, CA). Gene specific oligonucleotides for qPCR were obtained as a TaqMan® Gene Expression Assays by Applied Biosystems. The expression of CXCL14 and Rnase 7 as well as 18S and GAPDH housekeeping genes were determined. PB4522 induces less CXCL14 compared to Cetuximab and MEHD7945A. In contrast Rnase 7 expression was less severe hampered by PB4522 compared to Cetuximab and MEHD7945A.

### PB4522 Shows Superior ADCC Activity Compared to MEHD7945A

ADCC activity is an important anti-tumour mechanism of action for therapeutic antibodies in cancer. Human monoclonal antibodies directed to the HER family of receptors like cetuximab and trastuzumab induce ADCC.

Multiple strategies have been used to achieve ADCC enhancement including glycoengineering and mutagenesis. All of these seek to improve Fc binding to low-affinity activating FcγRIIIa and/or reducing binding to the low-affinity inhibitory FcγRIIIb. One of the methods used in glycoengineering to achieve ADCC enhancement is the removal of fucose. Removal of fucose has resulted in increased anti-tumour activity in several in vivo models [Junttila, 2010]. To maximize PB4522 activity, this afucosylation technology was applied (Liu and Lee. 2009 [13-17]) to remove fucose from the N-linked carbohydrate structure in the Fc region.

To determine ADCC activity of PB4522 in comparison to MEDH7945a and cetuximab the ADCC Reporter Bioassay (Promega) was used. Three different cell lines where tested; the EGFR amplified and high EGFR expressing head and neck cell line A431, the intermediate EGFR expressing lung cancer cell line A549 and the intermediate EGFR expressing pancreatic cancer cell line BxPC3.

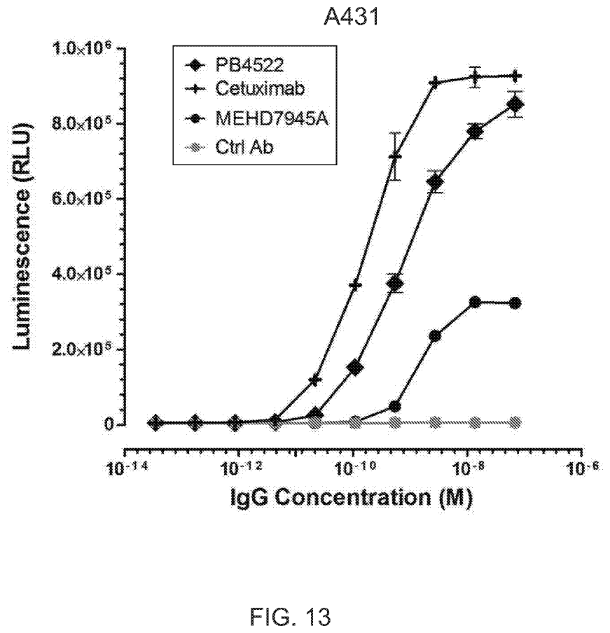

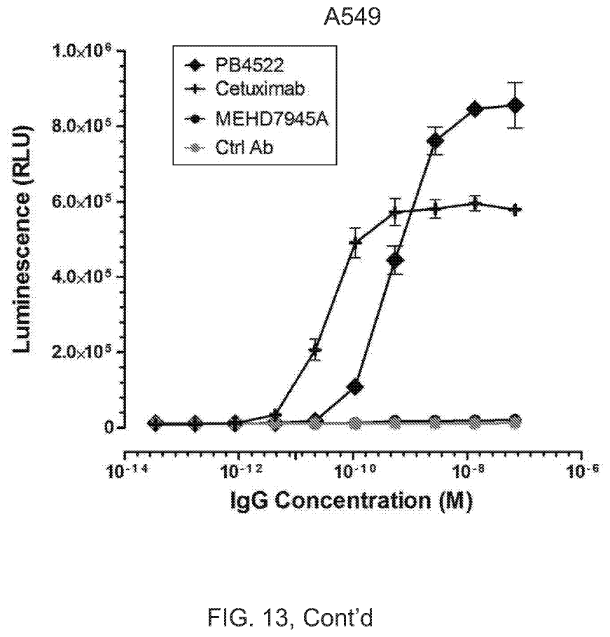

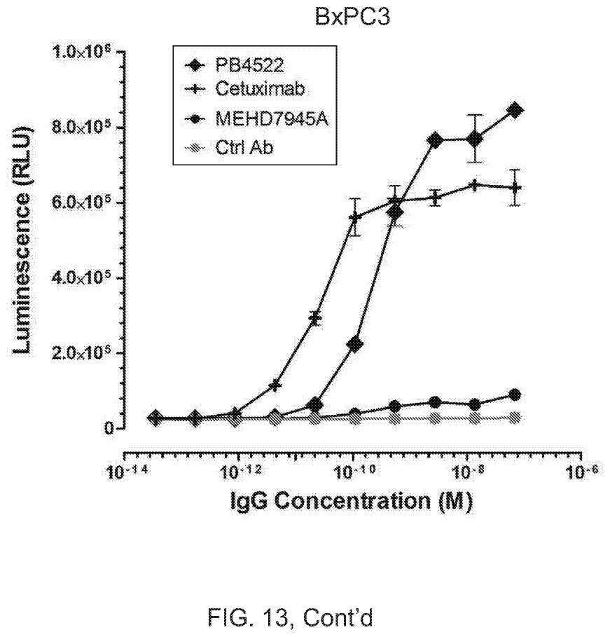

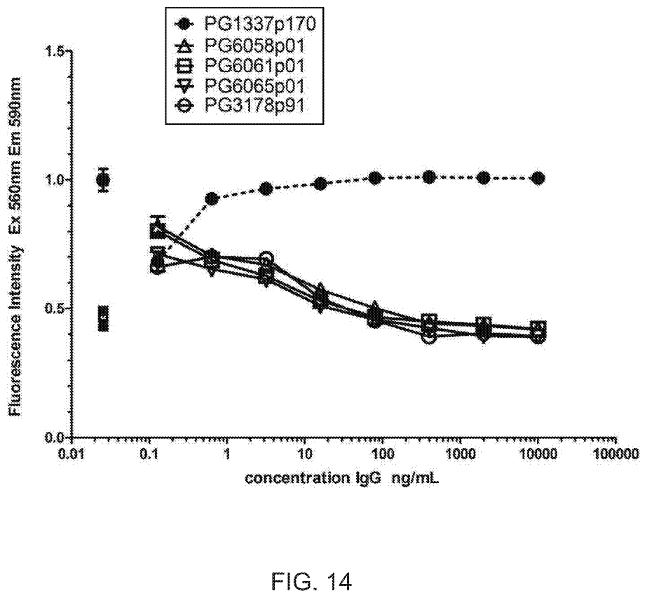

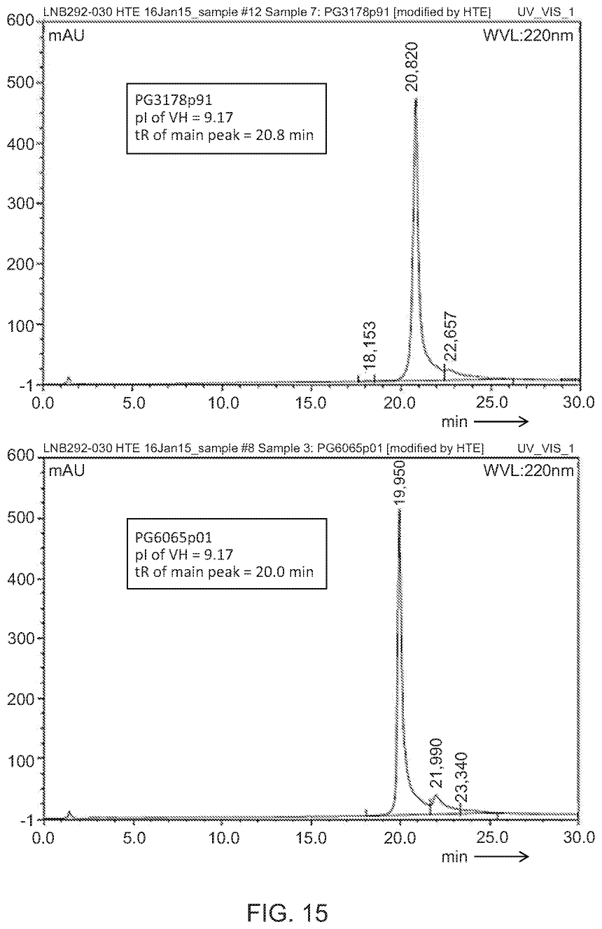

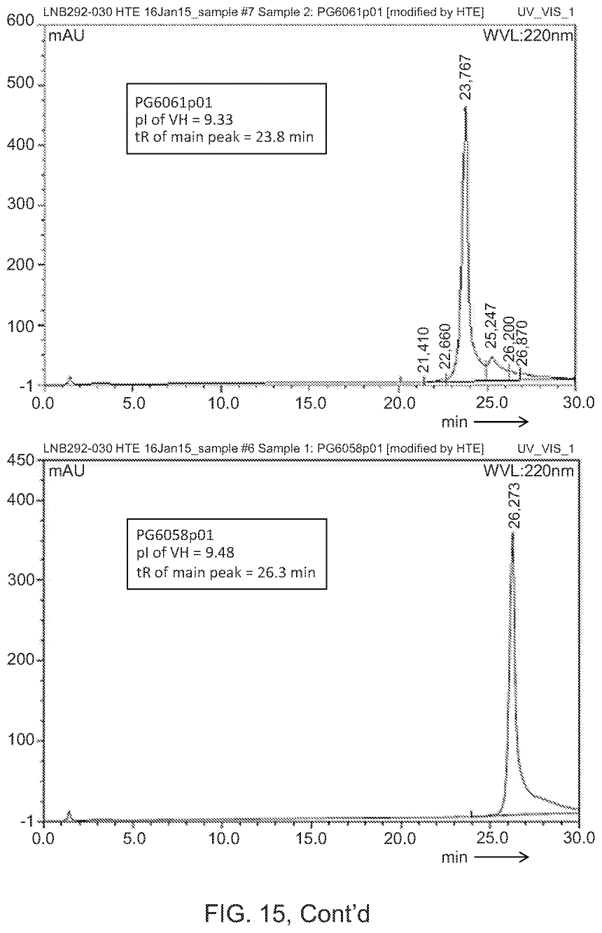

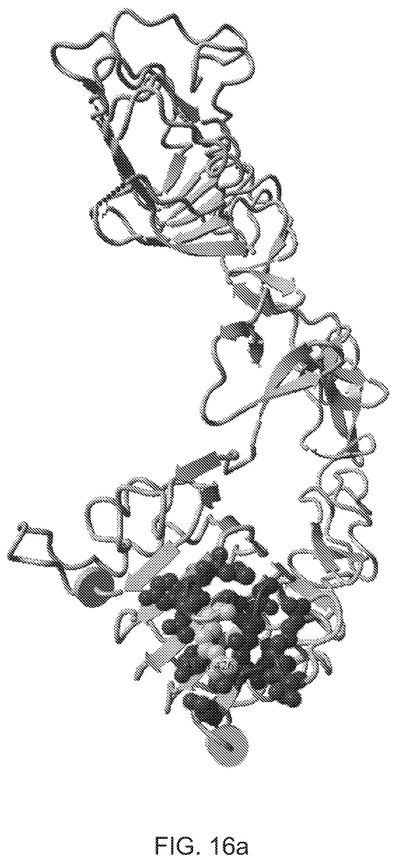

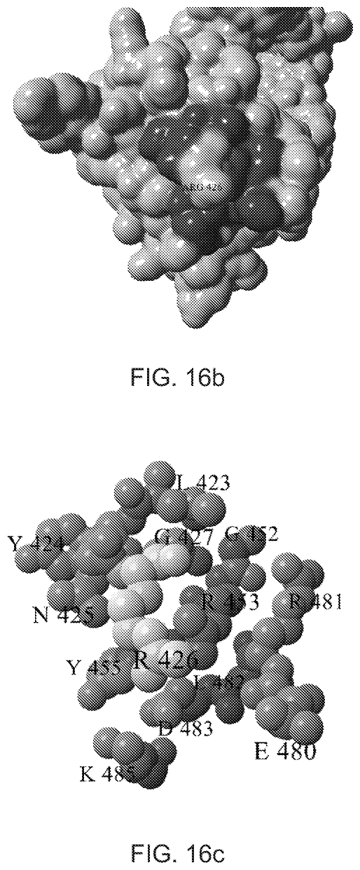

The bioassay uses engineered Jurkat cells stably expressing either the FcγRIIIa receptor V158 (high affinity) or F158 (low affinity) variant, and an NFAT response element driving expression of firefly luciferase which is a measure for FcγR activation. The assay has been validated by comparing data obtained with this ADCC Reporter Bioassay to the classical 51Cr release assay and both assays yield similar results. The ADCC assays were performed using the Promega ADCC Bioassay kit using 384 white well plates. In this experimental setup A431 cells, BxPC3 cells and A549 cells were plated at a density of 1000 cells/well in 30 μl assay medium (RPMI with 4% low IgG serum) 20-24 H before the bioassay. The next day, the culture medium was removed. Next, a serial dilution of antibodies, PB4522 and its comparator antibodies cetuximab, MEHD7945A and a Ctrl antibody were prepared in duplicates. 10 μl of these antibody dilutions were added to the wells. The starting concentrations of the antibodies were 10 μg/ml and a 10 points 5-fold serial dilutions were generated to provide full dose response curves. Finally, 5 μl of ADCC Bioassay effector cells (15000 cells/well, V158) were added. The cells were incubated for 6 H at 37° C. Next, 15 μl BIO-Glo luciferase substrate was added and 5 minutes later luminescence was detected in a plate reader. The obtained data are shown in FIG. 13. Both PB4522 and cetuximab showed ADCC activity towards the medium EGFR expressing cells BxPC3 and A549 whereby the EC50 of cetuximab was lower compared to PB4522. The total ADCC activity—Area Under the Curve (AUC)—and the maximal ADCC activity however of PB4522 were higher compared to cetuximab. All three antibodies showed ADCC activity towards the EGFR amplified cell line A431, whereby cetuximab showed the highest ADCC activity followed by PB4522 and Cetuximab. Of note is that the intermediate EGFR expressing cell lines are representative for the number of EGFR expressed on patient derived tumor cell samples. In all three cell lines the maximal ADCC and the AUC of PB4522 are higher compared to MEHD7945A. In all three cell lines the EC50 of PB4522 is lower compared to MEHD7945A.

### HER3

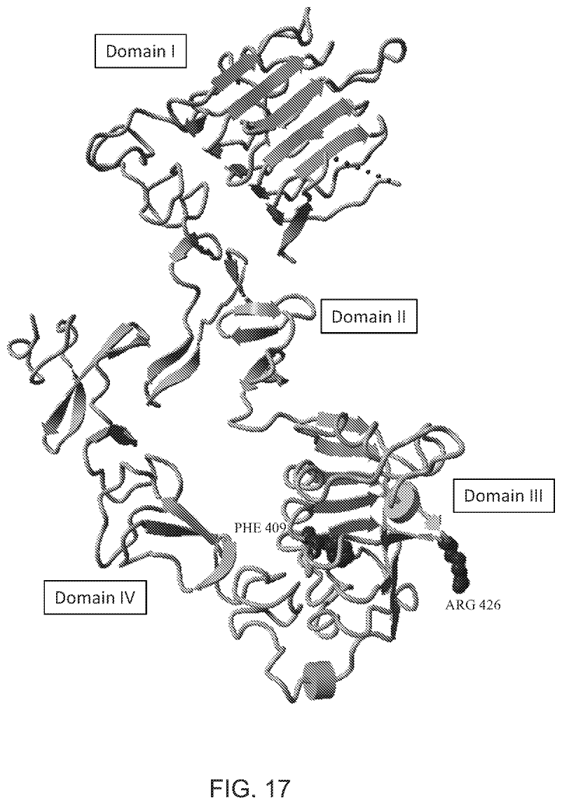

Binding analysis of PG3178 IgG at 0.25 μg/ml to HER3 ECD mutants in FACS resulted in the identification of two so-called ‘critical’ residues (F409, R426) for which mutation to alanine caused substantial loss of binding compared to WT HER3, while binding of the control mAb was retained (Table 7 and FIG. 17). Both residues are located in Domain III of HER3 and spatially distant. Moreover, F409 is buried in the HER3 hydrophobic core, which makes it unlikely to be part of the PG3178 epitope.

### Confirmation Experiments HER3 Epitope

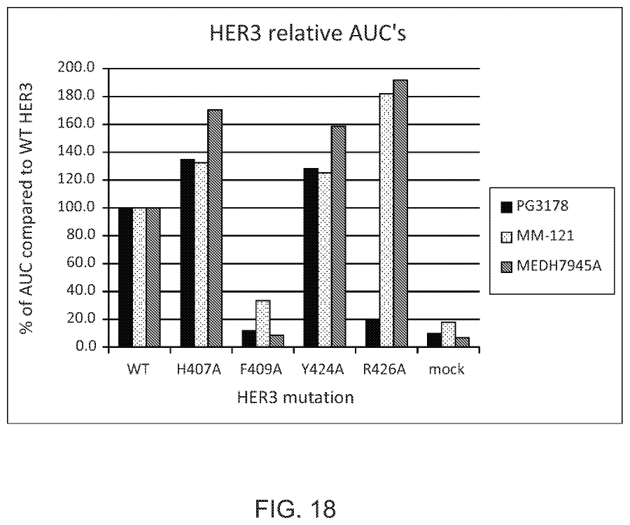

CHO-K1 cells were transfected with HER3 ECD mutation constructs (listed in Table 7), WT HER3 ECD and two control constructs (H407A and Y424A). PG3178 binding to the HER3 ECD variants was tested in a FACS titration experiment. Two control antibodies, binding Domain I (MM-121) and Domain III (MEHD7945A) of HER3 were included to verify HER3 ECD expression on the cell surface. Mean MFI values were plotted and for each curve the AUC was calculated using Graph Pad Prism 5 software. WT HER3 binding was used to normalize the data. The R426A mutation was shown to be critical for PG3178 binding whereas the binding to F409A could not be confirmed due to loss of cell surface expression (FIG. 18).

### REFERENCES CITED IN THE EXAMPLES

1. Schmitz, K. R. and K. M. Ferguson, Interaction of antibodies with ErbB receptor extracellular regions. Exp Cell Res, 2009. 315(4): p. 659-70.
2. de Haard, H. J., et al., A large non-immunized human Fab fragment phage library that permits rapid isolation and kinetic analysis of high attinity antibodies. J Biol Chem, 1999. 274(26): p. 18218-30.
3. Marks, J. D., et al., By passing immunization. Human antibodies from V-gene libraries displayed on phage. J Mol Biol, 1991. 222(3): p. 581-97.
4. Meulemans, E. V., et al., Selection of phage-displayed antibodies specific for a cytoskeletal antigen by competitive elution with a monoclonal antibody. J Mol Biol, 1994. 244(4): p. 353-60.
5. Giard, D. J., et al., In vitro cultivation of human tumors: establishment of cell lines derived from a series of solid tumors. J Natl Cancer Inst, 1973. 51(5): p. 1417-23.
6. Merlino, G. T., et al., Amplification and enhanced expression of the epidermal growth factor receptor gene in A431 human carcinoma cells. Science, 1984. 224(4647): p. 417-9.
7. Cochran, J. R., et al., Domain-level antibody epitope mapping through yeast surface display of epidermal growth factor receptor fragments. J Immunol Methods, 2004. 287(1-2): p. 147-58.
8. Ledon, N., et al., Comparative analysis of binding attinities to epidermal growth factor receptor of monoclonal antibodies nimotuzumab and cetuximab using different experimental animal models. Placenta, 2011. 32(7): p. 531-4.
9. Gulli, L. F., et al., Epidermal growth factor-induced apoptosis in A431 cells can be reversed by reducing the tyrosine kinase activity. Cell Growth Differ, 1996. 7(2): p. 173-8.
10. Pastore S, Mascia F, Mariotti F, Dattilo C, Mariani V, Girolomoni G. ERK1/2 regulates epidermal chemokine expression and skin inflammation. J Immunol. 2005; 174(8):5047-5056.
11. Lichtenberger B M, Gerber P a., Holcmann M, et al. Epidermal EGFR Controls Cutaneous Host Defense and Prevents Inflammation. Sci Transl Med. 2013; 5: 199ra111-199ra111. doi:10.1126/scitranslmed.3005886.
12. PCT/NL2013/050294
13. ADCC Enhancement Technologies for Next Generation Therapeutic Antibody. Cheng Liu and Andreia Lee. Antibody therapeutics—Trends in Bio/Pharmaceutical Industry 2009 [13-17]

TABLE 1
Overview of the phage antibody libraries generated from EGFR-
immunised mice.

| Library | | Library | Insert | Unique |
|---|---|---|---|---|
| no. | Mouse | size | frequency | clones |
| ML1155 | E094#12 and E094#18 | 8.30E+06 | 96% | 97% |
| ML1156 | E094#14 and E094#16 | 8.60E+06 | 100% | 97% |

TABLE 2
Overview of known anti-EGFR antibodies used to epitope map
the selected phage antibodies. N/A: not applicable.

| Antibody | | | Domain |
|---|---|---|---|
| name | PG nr/supplier: | Cat. Nr. | specificity |
| ICR10 | Abcam | ab321 | I |
| EGFR.1 | Thermo scientific | MS-311-P | II |
| MatuzuMab | PG2982p02 | n/a | III |
| Cetuximab | Merck (clinical batch) | n/a | III |

TABLE 3
Overview of the domain specificity (as assessed by FACS using the ‘swap domain’
mutants and functionality of anti-EGFR antibodies. Functionality was compared
to the clinically used antibody cetuximab. Activity of anti-EGFR cLC IgG (PG
codes) in inhibiting EGF-induced cell death in A431 cells, compared to the activity
of cetuximab in that assay. ND not determined. Variant III: EGFR variant III
specific. PG codes represent full length IgG1 monoclonal antibodies.

| | | | | | | EGFR |
|---|---|---|---|---|---|---|
| | | | | | | blocking |
| | Representative | | EGFR | Mouse | Cynomolgus | activity |
| Cluster | antibody | Origen of | domain | cross- | cross- | compared to |
| nr. | tested: | antibody | specificity | reactivity | reactivity | cetuximab |
| 1 | PG3998 | MeMo | III | No | Yes | >100% |
| | PG4010 | MeMo | | | | |
| 2 | PG4289 | MeMo | I | No | Yes | 80% |
| | PG4003 | MeMo | | | | |
| 3 | PG4000 | MeMo | IV | No | Yes | <5% |
| 4 | PG4016 | MeMo | I | No | Yes | <5% |
| 5 | PG4033 | MeMo | II | No | Yes | 0% |
| 6 | PG4034 | MeMo | II | No | Yes | 0% |
| 7 | PG4035 | MeMo | IV | No | Yes | 0% |
| 8 | PG4032 | MeMo | III | No | Yes | <5% |
| 9 | PG4284 | MeMo | III | No | Yes | 30% |
| 10 | PG4358 | MeMo | III | No | Yes | ND |
| 11 | PG4280 | MeMo | III | No | Yes | 100% |
| 12 | PG4283 | MeMo | Variant III | No | Yes | 0% |
| 13 | PG4281 | MeMo | III | No | Yes | 70% |
| 14 | PG4286 | MeMo | III/IV | No | Yes | <10% |
| 15 | PG4285 | MeMo | Variant III | No | Yes | 0% |
| 16 | PG4287 | MeMo | III | No | Yes | <10% |
| 17 | PG4359 | MeMo | ND | No | Yes | ND |
| 18 | PG3370 | Synthetic | III | Yes | Yes | 80% |
| | | library | | | | |

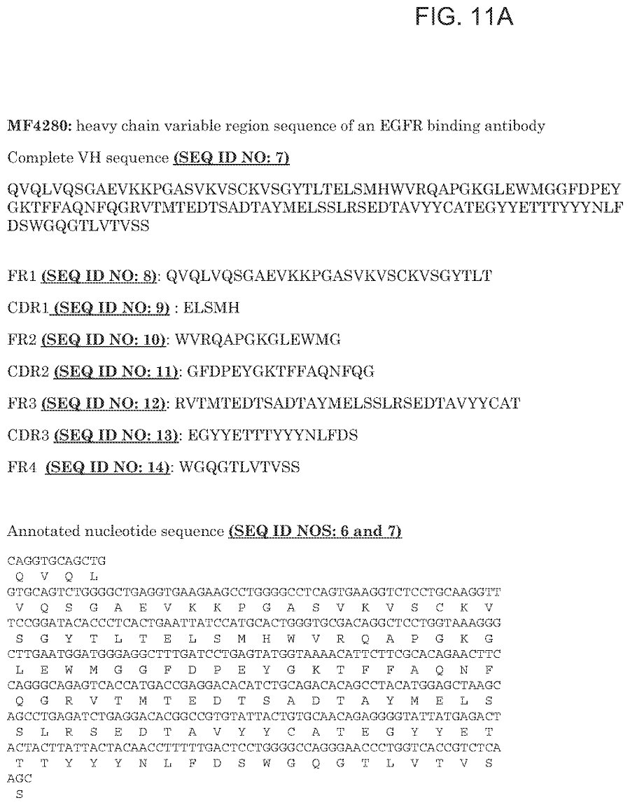

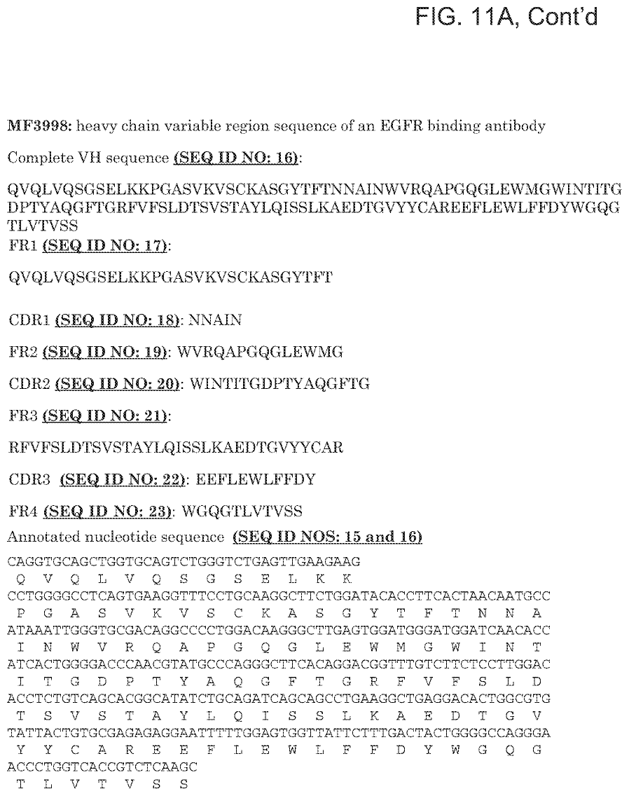

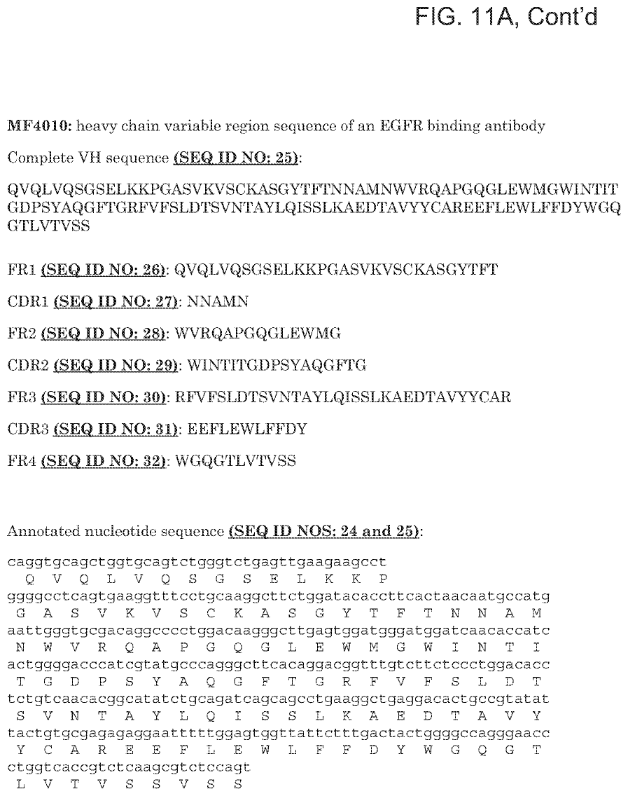

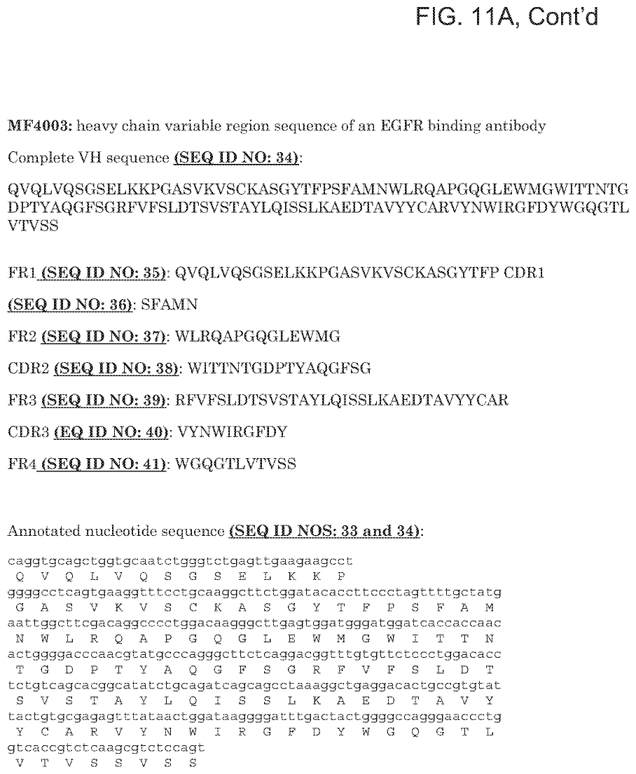

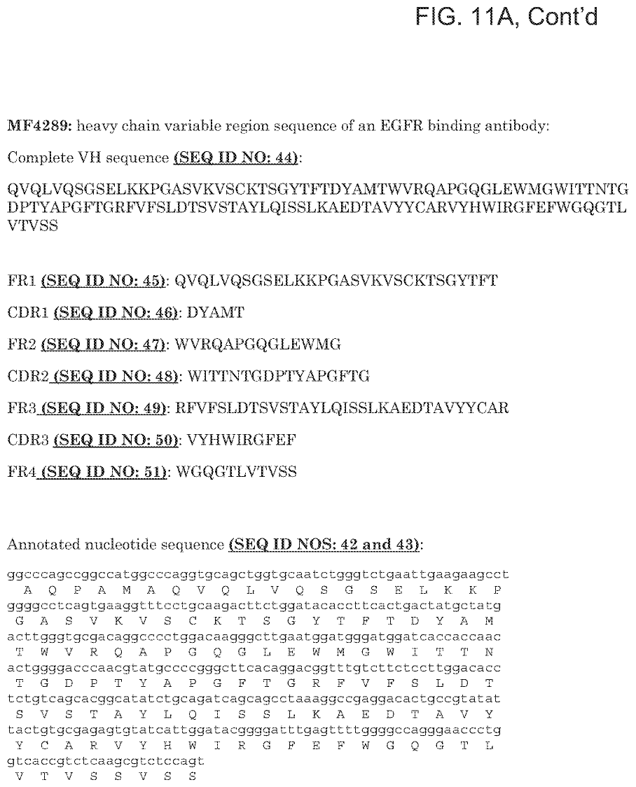

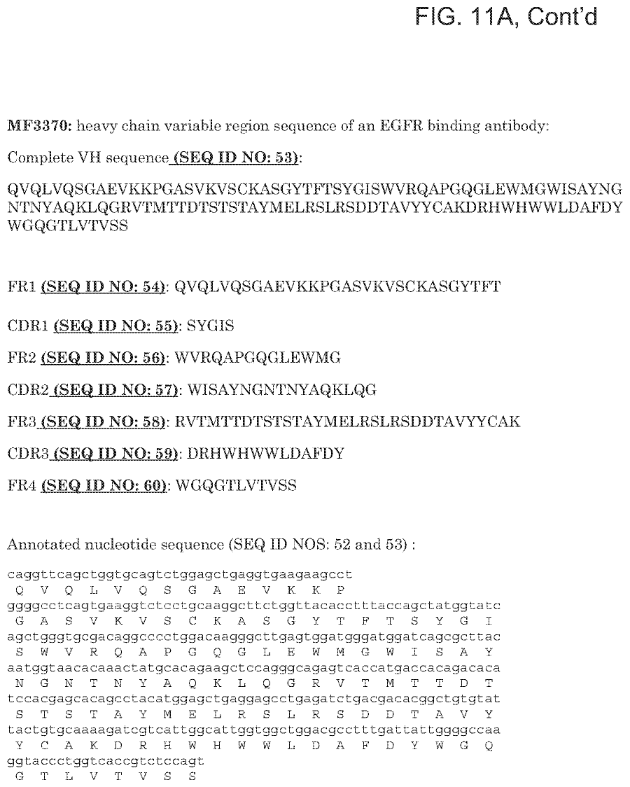

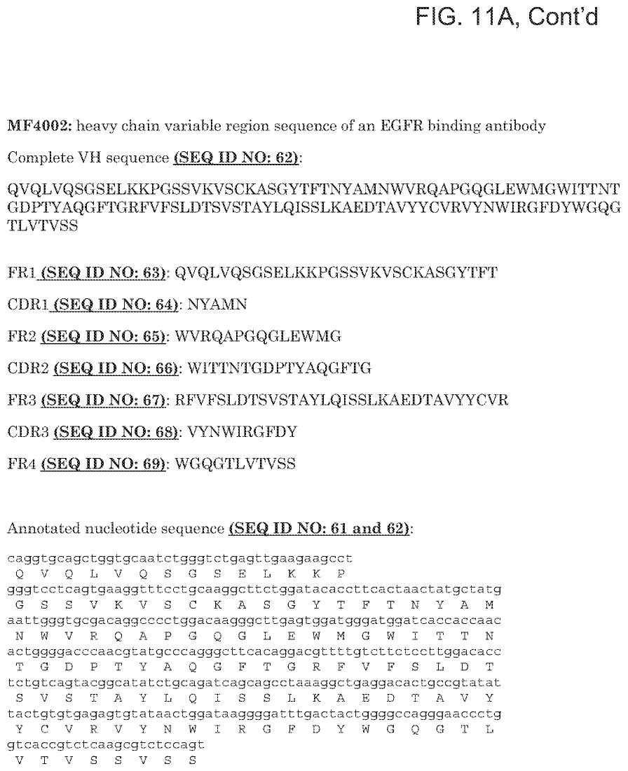

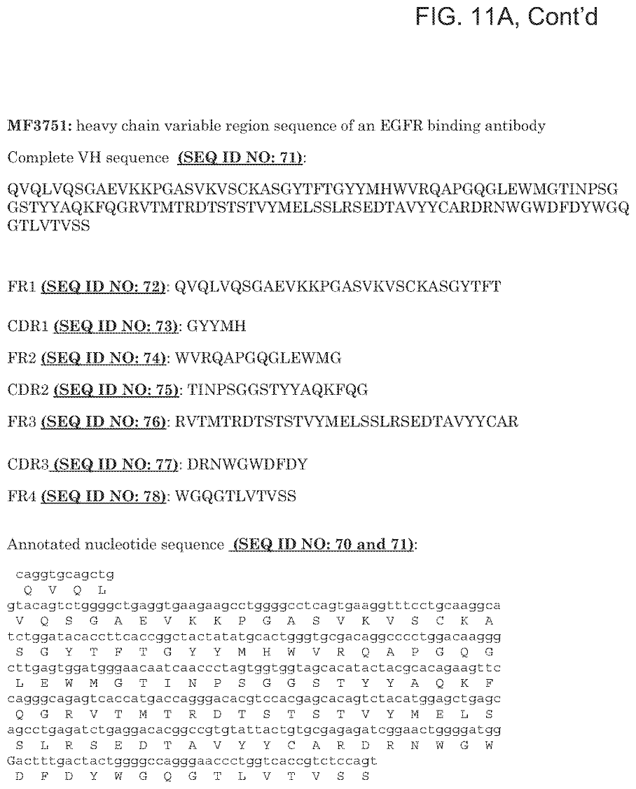

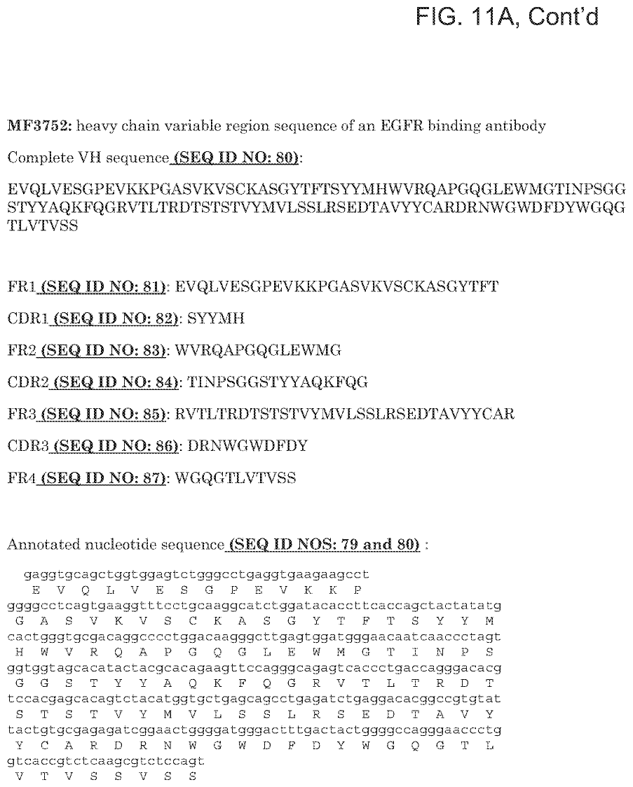

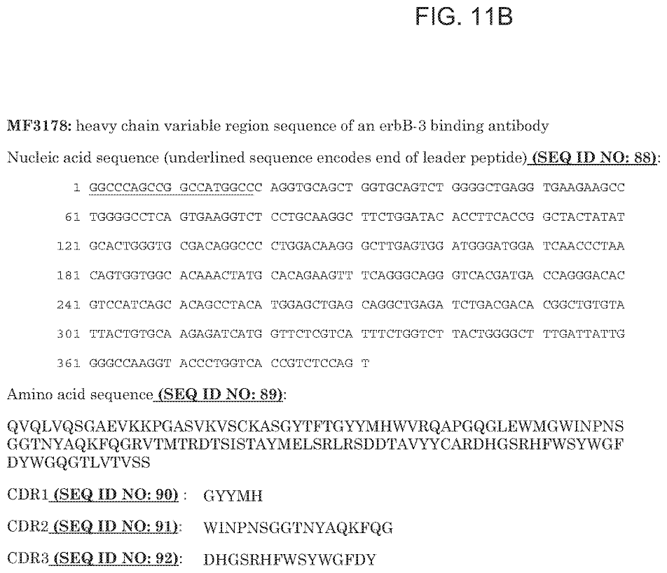

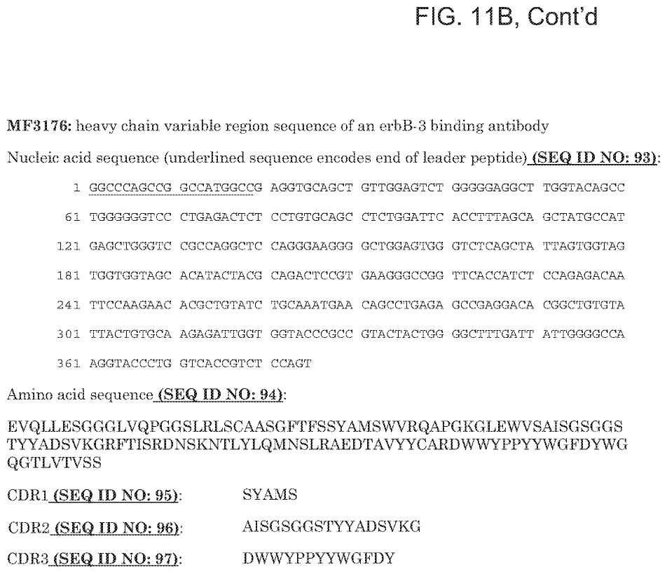

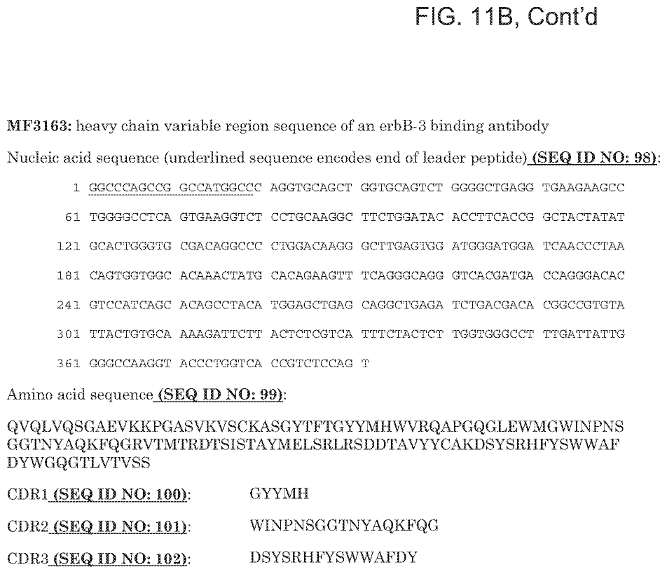

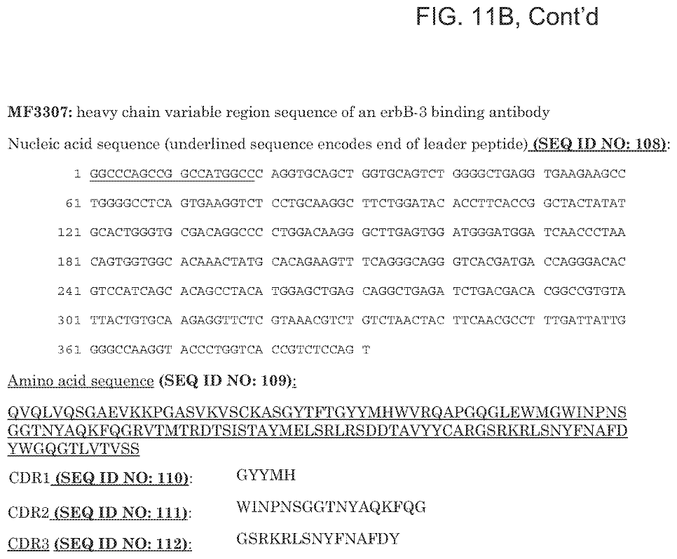

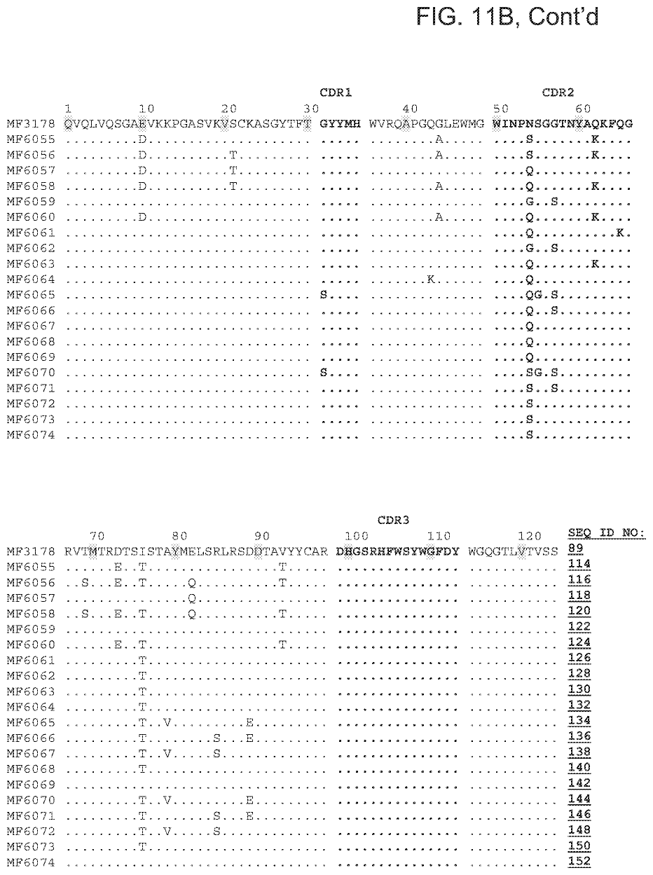

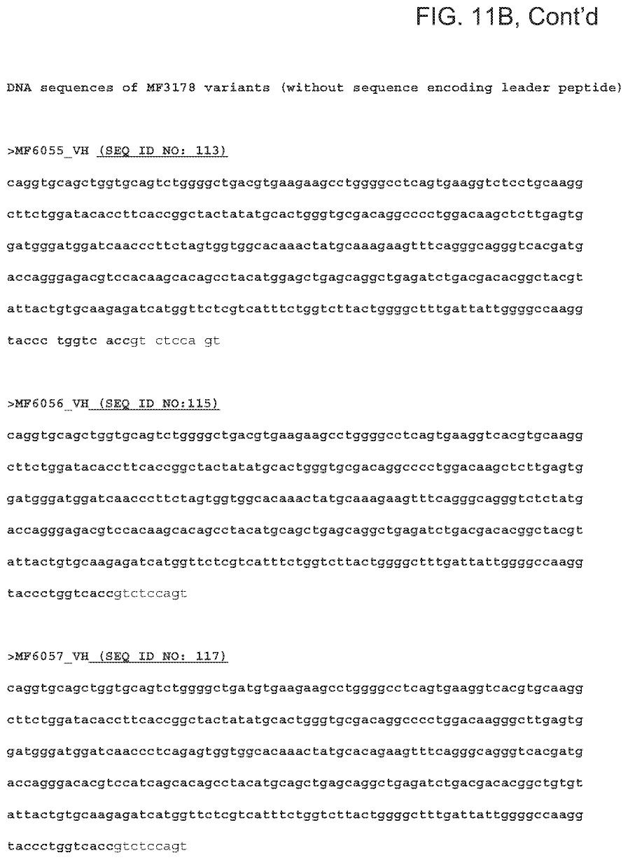

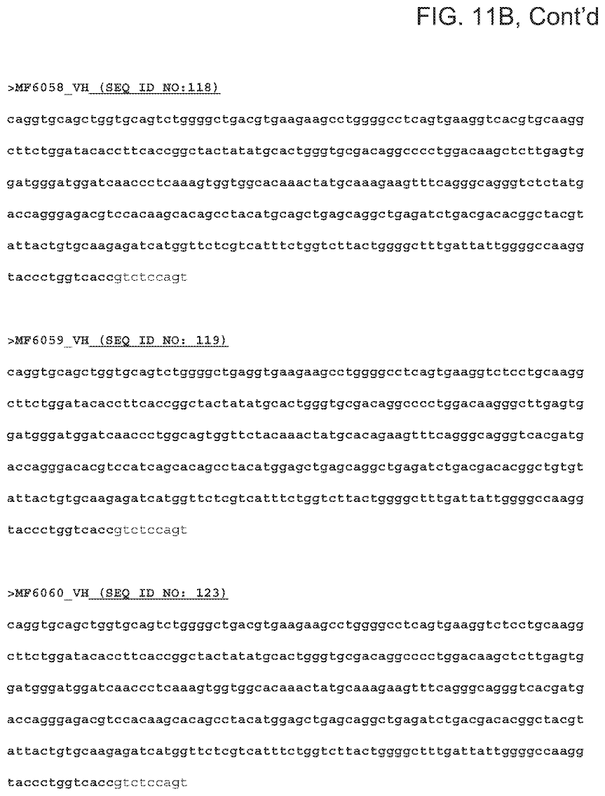

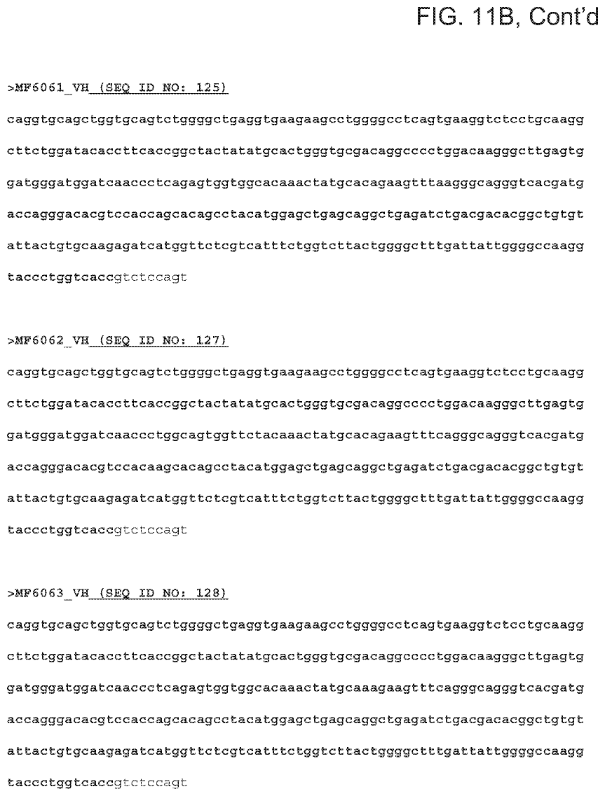

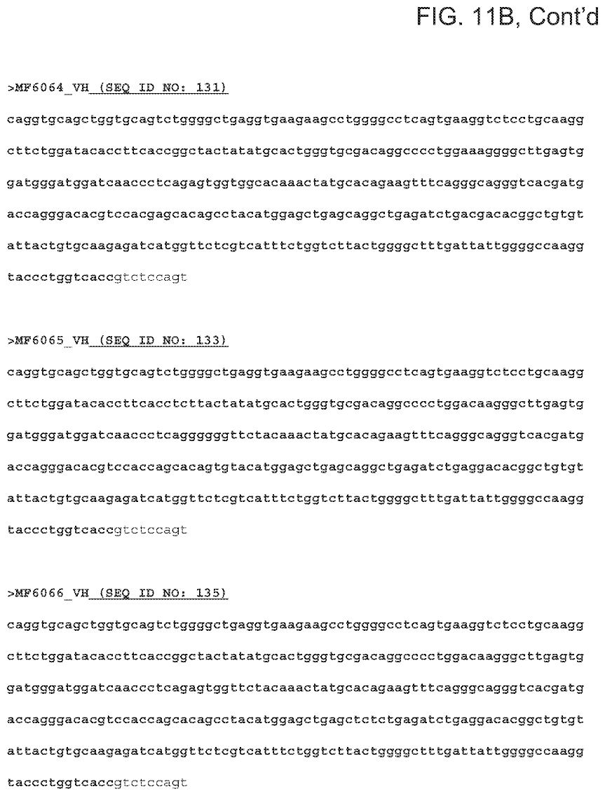

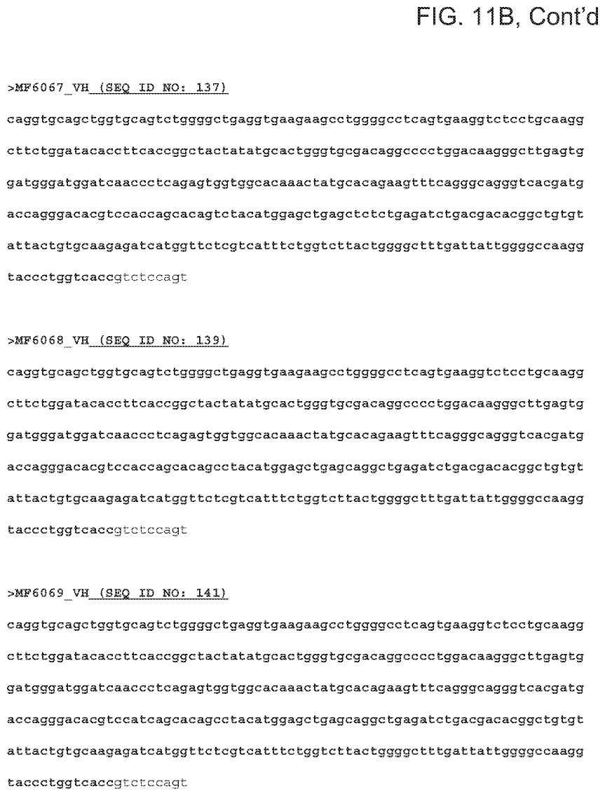

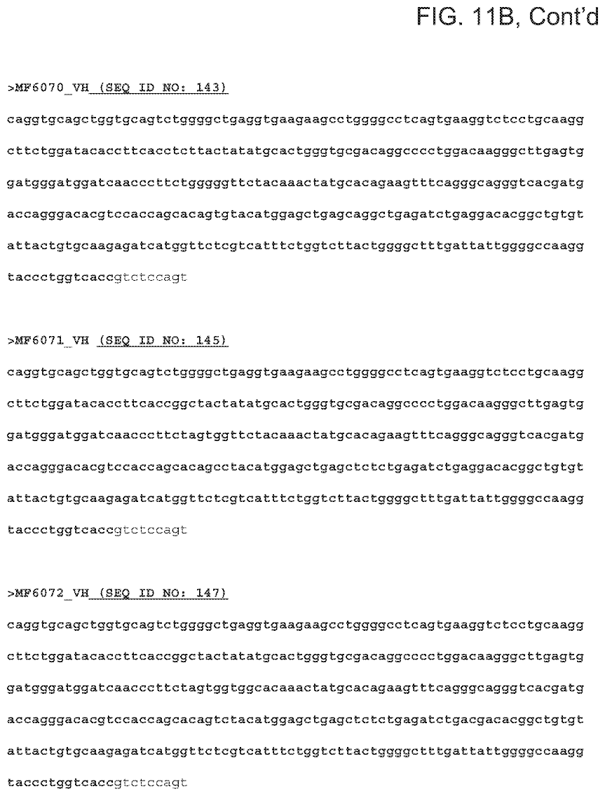

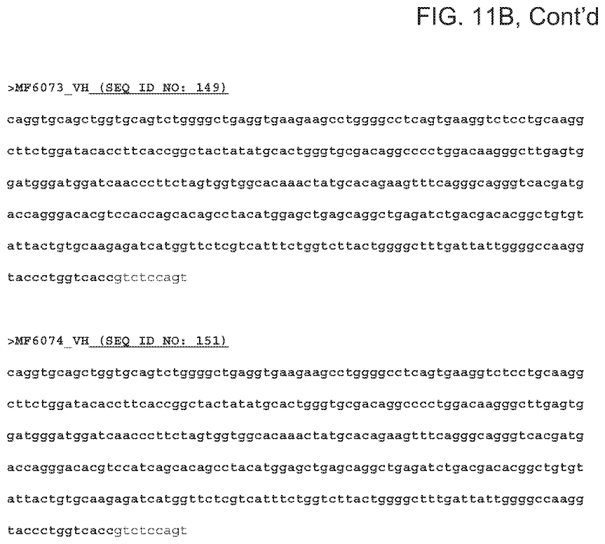

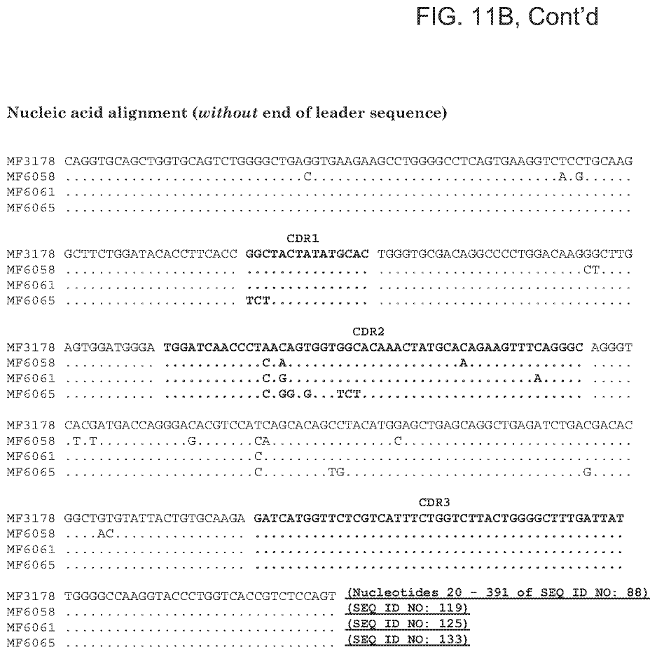

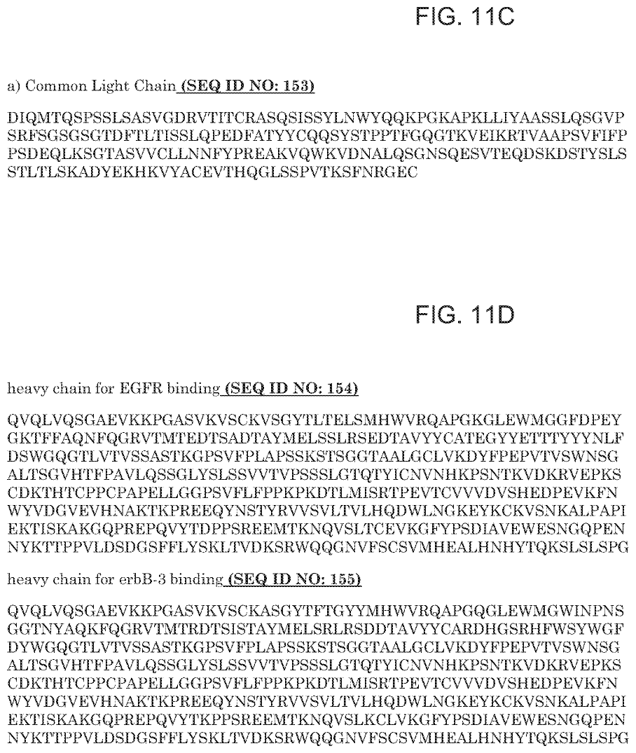

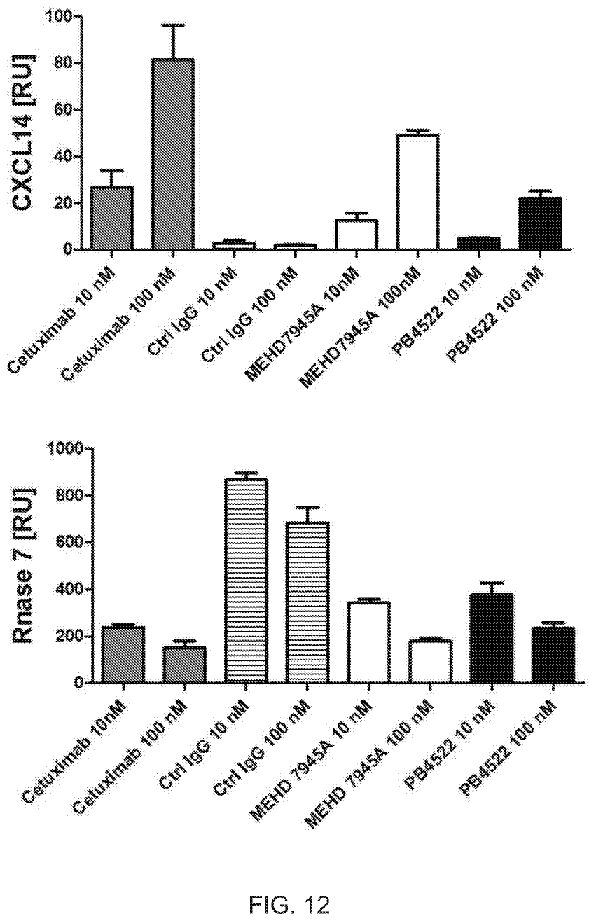

TABLE 4
List of antagonistic anti-EGFR × anti-HER3 bispecific antibodies
(PB codes) tested for BxPC3-Luc2 cell proliferation inhibition. These
anti-EGFR and anti-HER3 arms were all shown to be active as mono-specific
monoclonal antibody in inhibiting ligand (EGF- or NRG-) driven growth
of tumour cells. The table shows the number of the bispecific protein
(PB) that is composed of the respective EGFR- and HER3 binding arms (e.g.
PB4510 is composed of MG3998 and MG3178). The VH chain sequence of various
MG chains are indicated with MF followed by the number in FIGS. 11A-11D.
PB codes represent full length IgG1 bispecific antibodies

| | HER3 | | | | | |
|---|---|---|---|---|---|---|
| EGFR | MG3178 | MG3176 | MG3163 | MG3157 | MG3156 | MG3125 |
| MG3998 | PB4510 | PG4656 | PG4533 | PB4579 | PB4631 | PB4654 |
| MG3999 | PB4511 | PB4557 | PB4534 | PB4580 | PB4616 | PB4639 |
| MG4010 | PB4512 | PB4558 | PB4535 | PB4581 | PB4617 | PB4640 |
| MG4013 | PB4514 | PB4560 | PB4537 | PB4583 | PB4618 | PG4641 |
| MG3751 | PB4518 | PB4564 | PB4541 | PB4587 | PB4620 | PB4643 |
| MG3752 | PB4519 | PB4565 | PB4542 | PB4588 | PB4621 | PB4644 |
| MG4025 | PB4521 | PB4567 | PB4544 | PB4590 | PB4622 | PB4645 |
| MG4280 | PB4522 | PB4568 | PB4545 | PB4591 | PB4623 | PB4646 |
| MG4290 | PB4523 | PB4569 | PB4546 | PB4592 | PB4624 | PB4647 |
| MG4281 | PB4524 | PB4570 | PB4547 | PB4593 | PB4625 | PB4648 |
| MG4284 | PB4525 | PB4571 | PB4548 | PB4594 | PB4626 | PB4649 |
| MG3370 | PB4526 | PB4572 | PB4549 | PB4595 | PB4627 | PB4650 |
| MG4002 | PB4527 | PB4573 | PB4550 | PB4596 | PB4628 | PB4651 |
| MG4003 | PB4528 | PB4574 | PB4551 | PB4597 | PB4629 | PB4652 |
| MG4289 | PB4529 | PB4575 | PB4552 | PB4598 | PB4630 | PB4653 |
| MG4011 | PB4513 | PB4559 | PB4536 | PB4582 | PB4632 | PB4655 |
| MG4014 | PB4515 | PB4561 | PB4538 | PB4584 | PB4633 | PB4656 |
| MG3756 | PB4517 | PB4563 | PB4540 | PB4586 | PB4634 | PB4657 |
| MG4023 | PB4520 | PB4566 | PB4543 | PB4589 | PB4635 | PB4658 |

TABLE 5
List of six anti-EGFR × anti-HER3 bispecific antibodies that were
selected for in vivo testing in the BxPC3-Luc2 orthotopic model
and their IC50 value for ligand-driven BxPC-3 cell proliferation
inhibition.

| | | | | IC50 for BxPC-3 |
|---|---|---|---|---|
| | | | Domain | ligand-driven cell |
| | HER3 | EGFR | specificity | proliferation |
| PB nr. | arm | arm | EGFR arm | inhibition (pM) |
| PB4510 | MG3178 | MG3998 | Domain III | 7 |
| PB4522 | MG3178 | MG4280 | Domain III | 13 |
| PB4528 | MG3178 | MG4003 | Domain I | 29 |
| PB4535 | MG3163 | MG4010 | Domain III | 65 |
| PB4549 | MG3163 | MG3370 | Domain III | 250 |
| PB4552 | MG3163 | MG4289 | Domain I | 62 |
| MEHD7945A | N/A | N/A | Domain III | 260 |

TABLE 6
IC50 values for inhibition of BxPC-3 cell proliferation determined
for the different antibody formats tested: the bispecific (PB) anti-
EGFR × HER3 leads, or a mix of the mono-specific, bivalent
parental (PG) antibodies.

| | | | IC50 for BxPC-3 ligand- |
|---|---|---|---|
| Antibody | | | driven cell proliferation |
| tested | HER3 arm | EGFR arm | inhibition (pM) |
| PB4510 | MG3178 | MG3998 | 13 |
| PG3178 + | | | 260 |
| PG3998 | | | |
| PB4522 | MG3178 | MG4280 | 27 |
| PG3178 + | | | 390 |
| PG4280 | | | |
| PB4528 | MG3178 | MG4003 | 29 |
| PG3178 + | | | 80 |
| PG4003 | | | |
| PB4535 | MG3163 | MG4010 | 65 |
| PG3163 + | | | 670 |
| PG4010 | | | |
| PB4549 | MG3163 | MG3370 | 253 |
| PG3163 + | | | 753 |
| PG3370 | | | |
| PB4562 | MG3163 | MG4289 | 62 |
| PG3163 + | | | 767 |
| PG4289 | | | |
| MEHD7945A | N/A | N/A | 287 |

TABLE 7
The mean binding protein reactivities (and ranges) are listed for both
critical residues. Critical residues involved in PG3178 binding were
identified as those mutated in clones that were negative for PG3178
mAb binding (<20% WT) but positive for the control mAb 66223
binding (>70% WT). Residue numbering is that of PDB ID #4P59.

| | | | Control | |
|---|---|---|---|---|
| | | PG3178 | of mAB | |
| | | binding | binding | |
| | | % of wt | % of wt | |
| HER3 | | binding | binding | |
| Residue | Mutation | (range) | (range) | Designation |
| 409 | F409A | 16.74 (8) | 79.63 (0) | Possibly |
| | | | | critical |
| 426 | R426A | 3.17 (5) | 93.08 (36) | Critical |

TABLE 8
List of exposed residues within 11.2 Å radius of Arg 426 in HER3:

| | |
|---|---|
| Leu 423 | L423 |
| Tyr 424 | Y424 |
| Asn 425 | N425 |
| Gly 427 | G427 |
| Gly 452 | G452 |
| Arg 453 | R453 |
| Tyr 455 | Y455 |
| Glu 480 | E480 |
| Arg 481 | R481 |
| Leu 482 | L482 |
| Asp 483 | D483 |
| Lys 485 | K485 |

### REFERENCES CITED IN THE SPECIFICATION

1. Garrett T P, McKern N M, Lou M, Elleman T C, Adams T E, Lovrecz G O, Zhu H J, Walker F, Frenkel M J, Hoyne P A, Jorissen R N, Nice E C, et al. Crystal structure of a truncated epidermal growth factor receptor extracellular domain bound to transforming growth factor alpha. Cell 2002; 110:763-73.
2. Ogiso H, Ishitani R, Nureki O, Fukai S, Yamanaka M, Kim J H, Saito K, Sakamoto A, Inoue M, Shirouzu M, Yokoyama S. Crystal structure of the complex of human epidermal growth factor and receptor extracellular domains. Cell 2002; 110: 775-87.
3. Ferguson K M. Structure-based view of epidermal growth factor receptor regulation. Annu Rev Biophys 2008; 37: 353-73.
4. Yarden Y. The EGFR family and its ligands in human cancer. Signalling mechanisms and therapeutic opportunities. Eur J Cancer 2001; 37 (Suppl 4):S3-S8.
5. Jorissen R N, Walker F, Pouliot N, Garrett T P, Ward C W, Burgess A W. Epidermal growth factor receptor: mechanisms of activation and signalling. Exp Cell Res 2003; 284:31-53.
6. Buday L, Downward J. Epidermal growth factor regulates p21ras through the formation of a complex of receptor, Grb2 adapter protein, and Sos nucleotide exchange factor. Cell 1993; 73: 611-20.
7. Gale N W, Kaplan S, Lowenstein E J, Schlessinger J, Bar-Sagi D. Grb2 mediates the EGF-dependent activation of guanine nucleotide exchange on Ras. Nature 1993; 363:88-92.
8. Soltoff S P, Carraway K L, III, Prigent S A, Gullick W G, Cantley L C. ErbB3 is involved in activation of phosphatidylinositol 3-kinase by epidermal growth factor. Mol Cell Biol 1994; 14:3550-8.
9. Prigent S A, Gullick W J. Identification of cErbB-3 binding sites for phosphatidylinositol 30-kinase and SHC using an EGF receptor/c-ErbB-3 chimera. EMBO J 1994; 13:2831-41.
10. Uberall I, Kolar Z, Trojanec R, Berkovcova J, Hajduch M. The status and role of ErbB receptors in human cancer. Exp Mol Pathol 2008; 84:79-89.
11. Robertson S C, Tynan J, Donoghue D J. RTK mutations and human syndromes: when good receptors turn bad. Trends Genet 2000; 16:368.
12. Patel D K. Clinical use of anti-epidermal growth factor receptor monoclonal antibodies in metastatic colorectal cancer. Pharmacotherapy 2008; 28:31S-41S
13. Merchant et al. Nature Biotechnology, Vol. 16 July 1998 pp 677-681
14. Nissim A, Hoogenboom H R, Tomlinson I M, Flynn G, Midgley C, Lane D, Winter G. 1994. Antibody fragments from a ‘single pot’ phage display library as immunochemical reagents. EMBO J. 1994 Feb. 1; 13(3):692-8.
15. WO2004/009618
16. WO2009/157771
17. WO 2008/027236
18. WO 2010/108127
19. Schaefer et al. Cancer Cell 20, 472-486, October 2011
20. Olayioye M A et al.; EMBO J (2000) Vol 19: pp 3159-3167)
21. Kubota T, Niwa R, Satoh M, Akinaga S, Shitara K, Hanai N. Engineered therapeutic antibodies with improved effector functions. Cancer Sci. 2009 Sep: 100(9): 1566-72.
22 US Patent Application 20030078385
23. Gunasekaran (JBC 2010, vol 285, pp 19637-19646)
24. WO 2013/167954 A1

1. A bispecific antibody comprising a first antigen-binding site that binds EGFR and a second antigen-binding site that binds ErbB-3, wherein the antibody has a half maximal growth inhibitory concentration (IC50) of less than 200 pM for inhibiting EGFR and ErbB-3 ligand induced growth of BxPC3 cells (ATCC CRL-1687) or BxPC3-luc2 cells (Perkin Elmer 125058).

2. A bispecific antibody according to claim 1, that has an IC50 for inhibiting EGFR and ErbB-3 ligand induced growth of BxPC3 cells (ATCC CRL 1687) or BxPC3-luc2 cells (Perkin Elmer 125058) of less than 100 pM, preferably less than 50 pM, more preferably less than 20 pM.

3. A bispecific antibody according to claim 1 or claim 2, wherein said first antigen-binding site binds domain I or domain III of EGFR.

4. A bispecific antibody according to any one of claims 1-3, that can reduce a ligand-induced receptor function of ErbB-3 on an EGFR and ErbB-3 positive cell.

5. A bispecific antibody according to any one of claims 1-4, wherein said ligand induced BxPC3 cells are EGFR and ErbB-3 ligand induced BxPC3 cells.

6. A bispecific antibody according to any one of claims 1-5, wherein said ligand comprises EGF, neuregulin 1 or a combination thereof.

7. A bispecific antibody according to any one of claims 1-6, comprising a first antigen-binding site that binds EGFR and a second antigen-binding site that binds ErbB-3, wherein said second antigen-binding site binds domain III of ErbB-3.

8. A bispecific antibody according to any one of claims 1-7, wherein the antibody can reduce ligand-induced growth of an EGFR and ErbB-3 positive cell.

9. A bispecific antibody according to any one of claims 1-8, wherein the affinity (KD) of said second antigen-binding site for an ErbB-3 positive cell is lower than or equal to 2.0 nM, preferably lower than or equal to 1.39 nM, more preferably lower than or equal to 0.99 nM.

10. An antibody according to any one of claims 1-9, comprising an antigen-binding site that binds at least one amino acid of domain III of ErbB-3 selected from the group consisting and R426 and surface-exposed amino acid residues that are located within 11.2 Å from R426 in the native ErbB-3 protein.

11. An antibody according to any one of claims 1-10, comprising an amino acid sequence of FIG. 11.

12. An antibody according to any one of claims 1-11, wherein said antibody comprises at least the CDR3 sequence of an ErbB-3 specific heavy chain variable region selected from the group consisting of MF3178, MF3176, MF3163, MF3307, MF6055, MF6056, MF6057, MF6058, MF6059, MF6060, MF6061, MF6062, MF6068, MF6064, MF6065, MF6066, MF6067, MF6068, MF6069, MF6070, MF6071, MF6072, MF6073 and MF6074 as depicted in FIG. 11B.

13. A bispecific antibody according to claims 1-12, which exhibits antibody-dependent cell-mediated cytotoxicity (ADCC).

14. A bispecific antibody according to any one of claims 1-13, which is afucosylated in order to enhance ADCC.

15. A bispecific antibody according to any one of claims 1-14, that is a human or humanized antibody.

16. A bispecific antibody according to any one of claims 1-15, comprising two different immunoglobulin heavy chains with compatible hetero-dimerization domains.

17. A bispecific antibody according to claim 16, wherein said compatible hetero-dimerization domains are compatible immunoglobulin heavy chain CH3 hetero-dimerization domains.

18. A bispecific antibody according to claim 16 or claim 17, comprising two different immunoglobulin light chains with compatible hetero-dimerization domains.

19. A bispecific antibody according to any one of claims 1-18, comprising a common light chain, the light chain preferably comprises a light chain variable region IGKV1-39, preferably a germ-line IGKV1-39 variable region.

20. A pharmaceutical composition comprising a bispecific antibody according to any one of claims 1-19.

21. A bispecific antibody according to any one of claims 1-20, further comprising a label, preferably a label for in vivo imaging.

22. A method for the treatment of a subject having a EGFR, ErbB-3 or EGFR/ErbB-3 positive tumor or at risk of having said tumor comprising administering to the subject a bispecific antibody according to any one of claims 1-21.

23. A bispecific antibody according to any one of claims 1-21, for use in the treatment of a subject having or at risk of having an EGFR, ErbB-8 or EGFR/ErbB-3 positive tumor.

24. A bispecific antibody according to any one of claims 1-21 and 23, wherein said bispecific antibody induces less skin toxicity as compared to cetuximab.

25. A method for counteracting the formation of a metastasis in a subject having a EGFR, ErbB-8 or EGFR/ErbB-8 positive tumor, wherein said EGFR, ErbB-3 or EGFR/ErbB-3 positive tumor cell has a heregulin expression level that is at least 60%, preferably at least 70%, more preferably at least 80%, more preferably at least 85%, more preferably at least 90% or 95% of the heregulin expression level of BXPC3 or MCF7 cells, comprising administering to the subject a bispecific antibody comprising a first antigen-binding site that binds EGFR and a second antigen-binding site that binds ErbB-3.

26. A bispecific antibody comprising a first antigen-binding site that binds EGFR and a second antigen-binding site that binds ErbB-3 for use in the treatment or prevention of the formation of a metastasis of a EGFR, ErbB-3 or EGFR/ErbB-3 positive tumor cell, wherein said EGFR, ErbB-3 or EGFR/ErbB-3 positive tumor cell has a heregulin expression level that is at least 60%, preferably at least 70%, more preferably at least 80%, more preferably at least 85%, more preferably at least 90% or 95% of the heregulin expression level of BXPC3 or MCF7 cells.

27. A method or antibody for use according to any one of claims 22-26, wherein said subject has an EGFR or EGFR/ErbB-3 positive tumor that has less than 1.000.000 ErbB-2 cell-surface receptors per cell.

28. A method or antibody for use according to any one of claims 22-27, wherein said bispecific antibody is an antibody according to any one of claims 1-21 or 23.

29. A method or antibody for use according to any one of claims 22-28, wherein said tumor cell is a breast cancer cell, an ovarian cancer cell, a gastric cancer cell, a colorectal cancer cell, a pancreatic cancer cell or a lung cancer cell.
